SEQUENCE LISTING (70 sequences)

SEQ ID NO: 1 - DNA, 29 bp, Homo sapiens
gatctgtcaaatcgcctgcaggtaaaagc

SEQ ID NO: 2 - DNA, 32 bp, Homo sapiens
ttcttaaagatcaggttctgaagggtgatgga

SEQ ID NO: 3 - DNA, 31 bp, Homo sapiens
tgttatctgcttcctccaaccataaaacaaa

SEQ ID NO: 4 - DNA, 30 bp, Homo sapiens
ttcaatcattggttttctgcccattaggtt

SEQ ID NO: 5 - DNA, 30 bp, Homo sapiens
aaacggtttaccgccttccactcagagctc

SEQ ID NO: 6 - DNA, 31 bp, Homo sapiens
aactatgaagtgatgactgggtgagagagaa

SEQ ID NO: 7 - DNA, 32 bp, Homo sapiens
tatcagggttcttcagcgttgtgtattccttt

SEQ ID NO: 8 - DNA, 30 bp, Homo sapiens
tttttaacaagcatgggacacacaaagcaa

SEQ ID NO: 9 - DNA, 32 bp, Homo sapiens
cggaggtctttggccaactgctatagattttt

SEQ ID NO: 10 - DNA, 30 bp, Homo sapiens
ggtggtgaaactggatggaccatgaggatt

SEQ ID NO: 11 - DNA, 19 bp, Artificial Sequence
  Amplification primer
tacctgcaggcgatttgac

SEQ ID NO: 12 - DNA, 22 bp, Artificial Sequence
  Amplification primer
cacccttcagaacctgatcttt

SEQ ID NO: 13 - DNA, 21 bp, Artificial Sequence
  Amplification primer
tttatggttggaggaagcaga

SEQ ID NO: 14 - DNA, 20 bp, Artificial Sequence
  Amplification primer
aatgggcagaaaaccaatga

SEQ ID NO: 15 - DNA, 20 bp, Artificial Sequence
  Amplification primer
ctgagtggaaggcggtaaac

SEQ ID NO: 16 - DNA, 21 bp, Artificial Sequence
  Amplification primer
tctcacccagtcatcacttca

SEQ ID NO: 17 - DNA, 22 bp, Artificial Sequence
  Amplification primer
aatacacaacgctgaagaaccc

SEQ ID NO: 18 - DNA, 20 bp, Artificial Sequence
  Amplification primer
ttgtgtgtcccatgcttgtt

SEQ ID NO: 19 - DNA, 22 bp, Artificial Sequence
  Amplification primer
tctatagcagttggccaaagac

SEQ ID NO: 20 - DNA, 20 bp, Artificial Sequence
  Amplification primer
tcatggtccatccagtttca

SEQ ID NO: 21 - DNA, 34 bp, Homo sapiens
tttagcatgttcccaattctcaggaatttgtgtc

SEQ ID NO: 22 - DNA, 32 bp, Homo sapiens
cttttagttgctgctcttttccaggttcaagt

SEQ ID NO: 23 - DNA, 32 bp, Homo sapiens
ggctgctttgccctcagctcttgaagtaaacg

SEQ ID NO: 24 - DNA, 31 bp, Homo sapiens
tcttgtttttcaaattttgggcagcggtaat

SEQ ID NO: 25 - DNA, 34 bp, Homo sapiens
tgaatgcttctccaagaggcattgatattctctg

SEQ ID NO: 26 - DNA, 24 bp, Artificial Sequence
  Oligonucleotide probe
aaattcctgagaattgggaacatg

SEQ ID NO: 27 - DNA, 22 bp, Artificial Sequence
  Oligonucleotide probe
aacctggaaaagagcagcaact

SEQ ID NO: 28 - DNA, 22 bp, Artificial Sequence
  Oligonucleotide probe
acttcaagagctgagggcaaag

SEQ ID NO: 29 - DNA, 21 bp, Artificial Sequence
  Oligonucleotide probe
cgctgcccaaaatttgaaaaa

SEQ ID NO: 30 - DNA, 24 bp, Artificial Sequence
  Oligonucleotide probe
aatatcaatgcctcttggagaagc

SEQ ID NO: 31 - DNA, 628 bp, Homo sapiens
aaaaattgcaaccttccatttaaaatcagcttttatattgagtatttttttaaaatgttgtgtgtacatgctaggtgtgtatattaatttttatttgttacttgaaactaaactctgcaaatgcaggaaactatcagagtgatatctttgtcagtataaccaaaaaatatacgctatatctctataatctgttttacataatccatctatttttcttgatccatatgcttttacctgcaggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaa

SEQ ID NO: 32 - DNA, 628 bp, Homo sapiens
ggccaggaatttttgaatcagaatttttcttgttcgattttaatctcttatcatttagagattcttgaaatattgaaattactttgttcaaagtgaatgaattttcttaaattatgtatggttaacatcttttaaattgcttatttttaaattgccatgtttgtgtcccagtttgcattaacaaatagtttgagaactatgttggaaaaaaaaataacaattttattcttctttctccaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaaggtaattttattttctcaaatcccccagggcctgcttgcataaagaagtatatgaatctattttttaattcaatcattggttttctgcccattaggttattcatagttccttgctaaagtgtttttctcacaactttatttcttcttaaccctgcagttctgaaccagtgcacataagaacatatgtatatatgtgtgtgtgtgtatttatatatacacacacacatattgcatctataca

SEQ ID NO: 33 - DNA, 469 bp, Homo sapiens
tgtagggtggttggctaaaataattataattcctttaaaagaaattctacccactaaagttaatttagaagtaaaatataatagaaatccaataatatattcaccaaatggattaagatgttcatgaattatcttcaaagtgttaatcgaataagtaatgtgtatgcttttctgttaaagaggaagttagaagatctgagctctgagtggaaggcggtaaaccgtttacttcaagagctgagggcaaagcagcctgacctagctcctggactgaccactattggagcctgtaagtatactggatcccattctctttggctctagctatttgttcaaaagtgcaactatgaagtgatgactgggtgagagagaaaatttgtttcaattctaaagatagagataaacctttgtgttattgactgtgcaaaaagtcttagagtacattccttggaaattgactctgattcaa

SEQ ID NO: 34 - DNA, 598 bp, Homo sapiens
ccatttgagcctttaaatgaagaaaatctatagtcaagattttcatttgaaatatttttgatatctaagaatgaaacatatttcctgttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacagaggcgtccccagttggaagaactcattaccgctgcccaaaatttgaaaaacaagaccagcaatcaagaggctagaacaatcattacggatcgaagtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacatttttaaatcaattcagggcttatatagttgcaaagcatgc

SEQ ID NO: 35 - DNA, 515 bp, Homo sapiens
aaaggtgggttaccttatactgtcatgattgactaaatcatatggtaggttaaaagcaatctaatatatgtattctgacctgaggattcagaagctgtttacgaagtattttaagacactccaactagagatttcataaaaaaaactgacattcattctctttctcataaaaatctatagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaaggtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaac

SEQ ID NO: 36 - DNA, 32 bp, Homo sapiens
aaataggtctggcctaaaacacatacacatac

SEQ ID NO: 37 - DNA, 32 bp, Homo sapiens
gatcctgaaggttggtaaatttctggactacc

SEQ ID NO: 38 - DNA, 30 bp, Homo sapiens
ataatgtcaatccgacctgagctttgttgt

SEQ ID NO: 39 - DNA, 30 bp, Homo sapiens
tgtacaaggaccgacaagggtaggtaacac

SEQ ID NO: 40 - DNA, 34 bp, Homo sapiens
ctggagttcctgtaagataccaaaaaggcaaaac

SEQ ID NO: 41 - DNA, 29 bp, Homo sapiens
ctacaggaaaaattgggaagcctgaatct

SEQ ID NO: 42 - DNA, 30 bp, Homo sapiens
tgctatttcagtttcctggggaaaagaacc

SEQ ID NO: 43 - DNA, 34 bp, Homo sapiens
tctagcaatatccattacctcataatgggttatg

SEQ ID NO: 44 - DNA, 34 bp, Homo sapiens
ccacaggttgtgtcaccagagtaacagtctgagt

SEQ ID NO: 45 - DNA, 31 bp, Homo sapiens
ttataaaatcacagagggtgatggtgggtga

SEQ ID NO: 46 - DNA, 22 bp, Artificial
  Amplification primer
tgtatgtgttttaggccagacc

SEQ ID NO: 47 - DNA, 22 bp, Artificial
  Amplification primer
tccagaaatttaccaaccttca

SEQ ID NO: 48 - DNA, 20 bp, Artificial
  Amplification primer
aaagctcaggtcggattgac

SEQ ID NO: 49 - DNA, 20 bp, Artificial
  Amplification primer
acctacccttgtcggtcctt

SEQ ID NO: 50 - DNA, 24 bp, Artificial
  Amplification primer
gcctttttggtatcttacaggaac

SEQ ID NO: 51 - DNA, 19 bp, Artificial
  Amplification primer
caggcttcccaatttttcc

SEQ ID NO: 52 - DNA, 20 bp, Artificial
  Amplification primer
ttttccccaggaaactgaaa

SEQ ID NO: 53 - DNA, 24 bp, Artificial
  Amplification primer
cccattatgaggtaatggatattg

SEQ ID NO: 54 - DNA, 24 bp, Artificial
  Amplification primer
gactgttactctggtgacacaacc

SEQ ID NO: 55 - DNA, 21 bp, Artificial
  Amplification primer
caccatcaccctctgtgattt

SEQ ID NO: 56 - DNA, 32 bp, Homo sapiens
tgactgctggcaaaccacactattccagtcaa

SEQ ID NO: 57 - DNA, 30 bp, Homo sapiens
tttttcccattggaaatcaagctgggagag

SEQ ID NO: 58 - DNA, 32 bp, Homo sapiens
tcttccccagttgcattcaatgttctgacaac

SEQ ID NO: 59 - DNA, 33 bp, Homo sapiens
ttagacaaaatctcttccacatccggttgttta

SEQ ID NO: 60 - DNA, 31 bp, Homo sapiens
ccagtcggtaagttctgtccaagcccggttg

SEQ ID NO: 61 - DNA, 22 bp, Artificial
  Oligonucleotide probe
tggaatagtgtggtttgccagc

SEQ ID NO: 62 - DNA, 20 bp, Artificial
  Oligonucleotide probe
ccagcttgatttccaatggg

SEQ ID NO: 63 - DNA, 22 bp, Artificial
  Oligonucleotide probe
cagaacattgaatgcaactggg

SEQ ID NO: 64 - DNA, 23 bp, Artificial
  Oligonucleotide probe
aaccggatgtggaagagattttg

SEQ ID NO: 65 - DNA, 21 bp, Artificial
  Oligonucleotide probe
gggcttggacagaacttaccg

SEQ ID NO: 66 - DNA, 169 bp, Homo sapiens
atgtgtgtatgtgtatgtgttttaggccagacctatttgactggaatagtgtggtttgccagcagtcagccacacaacgactggaacatgcattcaacatcgccagatatcaattaggcatagagaaactactcgatcctgaaggttggtaaatttctggactaccact

SEQ ID NO: 67 - DNA, 223 bp, Homo sapiens
ctgttttaaaatttttatattacagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggtaggtaacacatatatttttcttg

SEQ ID NO: 68 - DNA, 226 bp, Homo sapiens
gttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaat

SEQ ID NO: 69 - DNA, 262 bp, Homo sapiens
gatctgcaatacatgtggagtctccaagggtatatttaaatttagtaattttattgctaactgtgaagttaatctgcactatatgggttcttttccccaggaaactgaaatagcagttcaagctaaacaaccggatgtggaagagattttgtctaaagggcagcatttgtacaaggaaaaaccagccactcagccagtgaaggtaatgaagcaacctctagcaatatccattacctcataatgggttatgcttcccctgttg

SEQ ID NO: 70 - DNA, 283 bp, Homo sapiens
tttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttg

(19) United States
(12) Patent Application Publication
(10) Pub. No.: US 2023/0109870 A1
(43) Pub. Date: Apr. 13, 2023

(54) METHODS AND TOOLS FOR ANALYSING THE DUCHENNE MUSCULAR DYSTROPHY (DMD) GENE

(71) Applicants: Centre Hospitalier Universitaire de Liège, Liège (BE); Université de Liège, Liège (BE)

(72) Inventors: Pablo Beckers, Liège (BE); François Boemer, Liège (BE); Laurent Servais, Liège (BE); Vincent Bours, Liège (BE)

(21) Appl. No.: 17/914,218

(22) PCT Filed: Mar. 25, 2021
(86) PCT No.: PCT/EP2021/057825

§ 371 Date: Sep. 23, 2022

(30) Foreign Application Priority Data
Mar. 25, 2020 (EP) 20165677.4

(51) Int. Cl. C12Q 1/6883

(52) U.S. Cl. CPC C12Q 1/6883; C12Q 2600/156; C12Q 2600/16

(57) ABSTRACT

An aspect of the invention provides a method for analysing the Duchenne Muscular Dystrophy (DMD) gene in a sample containing genetic material of a subject, wherein the method detects the presence or absence of at least exons 7, 43, 44, 45, 46, 49, 50, 51, 52 and 54 of the DMD gene in the genetic material of the subject, wherein the detection of the presence or absence of the exons comprises multiplex polymerase-based nucleic acid amplification. Another aspect of the invention provides a method for analysing the DMD gene in a sample containing genetic material of a subject, wherein the method detects the presence or absence of at least exons 7, 43, 44, 45, 46, 49, 50, 51, 52 and 54 of the DMD gene in the genetic material of the subject, and wherein the sample is blood. Additional aspects of the invention provide tools applicable in the disclosed methods, such as primers, primer pairs, primer pair sets, oligonucleotide probes and probe sets, as well as compositions and kits containing the same.

### FIELD

The invention is broadly in the medical or veterinary diagnosis field, and more particularly pertains to methods for detecting mutations in a gene responsible for Duchenne Muscular Dystrophy, as well as to tools and reagents useful in such methods, including amplification primers and primer pairs suitable for polymerase-based nucleic acid amplification of DMD gene segments, oligonucleotide probes, compositions, and kits.

### BACKGROUND

Duchenne Muscular Dystrophy (DMD) is the most common and severe form of muscular dystrophy, marked by progressive muscle degeneration with an incidence evaluated at about of 1/5000 males in the general population. The disorder is X-linked recessive. DMD is caused by mutations in the DMD gene, which encodes for the protein, dystrophin. Dystrophin interacts with other proteins to maintain the integrity and structure of musculoskeletal fibres in skeletal and cardiac (i.e., striated) muscle. In DMD patients, mutations of the DMD gene lead to a complete lack of dystrophin production.

Involvement of striated muscle begins in early childhood, generally before age of three years and is predominantly observed in males. Becker Muscular Dystrophy (BMD) also involves the DMD gene, and is characterised by residual dystrophin production, so that BMD patients present later and a milder clinical phenotype.

Deletions of one or more exons of the DMD gene account for approximately 60-70% of pathogenic variants in patients with DMD and BMD. Recently, novel therapeutic approaches to DMD involving exon skipping have been developed to induce the synthesis of a truncated and partially functional dystrophin protein. Eteplirsen (Exondys®) has been approved by the Food and Drug Administration (FDA) in 2016 for the treatment of DMD in patients who carry a confirmed mutation that is amenable to exon 51 skipping. Several other similar approaches, such as, suvodirsen, golodirsen, viltolarsen, casimersen, or rAAV-U7snRNA-E53, targeting exons 51, 53 or 45, are currently under evaluation to expand the spectrum of treatable DMD patients.

Apart from exon-skipping approaches, several gene therapy strategies are also under investigation. Ataluren (PTC124), a drug enabling ribosomal read-through of premature nonsense mutations to produce full-length, functional dystrophin, has shown promising results in several studies. Approved by the European Medicines Agency (EMA), ataluren is eligible for approximately 13% of DMD patients. Gene therapy using microdystrophin has proven efficacy in different canine models of DMD. Three phase 1/2 clinical trials are currently ongoing to assess safety of AAV-microdystrophin intravenous injection (ClinicalTrials identifier: NCT03368742, NCT03769116, NCT03362502). Many other downstream therapeutic alternatives are currently under investigation, such as, upregulation of utrophin, using GALGT2 gene therapy, idebenone, givinostat, or edasalonexent.

Considering the current advances in DMD treatment, implementation of comparatively widespread DMD screening such as particularly new-born screening (NBS) for DMD is increasingly being contemplated. To date, technical aspects of population-based screening for DMD have been evaluated in a few pilot studies. These assessments systematically considered the quantification of creatine kinase (CK), or its muscular isoform, CKMM, on dried blood spots (DBS) as a primary marker of DMD. However, CK is a marker of the disease process and does not directly reflect the genetic defect, and both false negatives and false positives occur using either CK or CKMM assays as a first-line test. Accordingly, the sensitivity and specificity of CK- or CKMM-based tests tends to be unsatisfactory. For example, the Wales DMD new-born screening program showed a sensitivity of 81.6% and positive predictive value of 38.6% (Moat et al. Eur J Hum Genet. 2013, vol. 21(10), 1049-1053).

### SUMMARY

Having recognised the need for novel ways to detect or screen for Duchenne Muscular Dystrophy (DMD), the present inventors herein describe improved methods and tools to evaluate the underlying genetic defects in the DMD gene. In particular, the present methods and tools advantageously allow to detect the presence or absence, such as a deletion or partial deletion, of at least exons 44, 46, 50, 52 and 54 and/or at least exons 7, 43, 45, 49 and 51 of the DMD gene, preferably at least exons 7, 43, 44, 45, 46, 49, 50, 51, 52 and 54 of the DMD gene. Deletions of one or more of these exons can be amenable to currently available or emerging exon skipping therapies, in particular those targeting DMD exons 8, 44, 50, 51, 45 or 53, and hence the present methods provide highly valuable information on the potential occurrence of such treatable DMD mutations. The methods may further optionally detect one or more additional genetic alterations in the DMD gene, in particular one or more pathological mutations in the DMD gene.

In certain aspects, the detection of the presence or absence of DMD exons 44, 46, 50, 52 and 54 and/or DMD exons 7, 43, 45, 49 and 51, preferably DMD exons 7, 43, 44, 45, 46, 49, 50, 51, 52 and 54 (and optionally of the one or more additional DMD genetic alterations) may comprise multiplex polymerase-based nucleic acid amplification. Notwithstanding the considerable advances in nucleic acid amplification technologies such as polymerase chain reaction (PCR) over the past decades, the design and operation of reliable and robust multiplexed amplification reactions of a plurality of nucleic acid target sequences of interest remains a formidable challenge with uncertain outcomes, which has been successfully tackled by the present inventors. Accordingly, an aspect of the invention provides a method for analysing the Duchenne Muscular Dystrophy (DMD) gene in a sample containing genetic material of a subject, wherein the method detects the presence or absence of at least exons 44, 46, 50, 52 and 54 and/or at least exons 7, 43, 45, 49 and 51 of the DMD gene, preferably at least exons 7, 43, 44, 45, 46, 49, 50, 51, 52 and 54 of the DMD gene in the genetic material of the subject, wherein the detection of the presence or absence of the exons comprises multiplex polymerase-based nucleic acid amplification.

The present inventors further realised and disclose the feasibility of detecting the presence or absence of at least exons 44, 46, 50, 52 and 54 and/or at least exons 7, 43, 45, 49 and 51 of the DMD gene, preferably at least exons 7, 43, 44, 45, 46, 49, 50, 51, 52 and 54 of the DMD gene in samples containing blood. By means of an example, blood is a particularly preferred sample material in the context of neonatology, where blood spot screening (heel prick test) is routinely performed in new-borns typically at or as soon as possible after 48 hours of age to assess a series of rare but serious health conditions. Accordingly, another aspect of the invention provides a method for analysing the DMD gene in a sample containing genetic material of a subject, wherein the method detects the presence or absence of at least exons 44, 46, 50, 52 and 54 and/or at least exons 7, 43, 45, 49 and 51 of the DMD gene, preferably at least exons 7, 43, 44, 45, 46, 49, 50, 51, 52 and 54 of the DMD gene in the genetic material of the subject, and wherein the sample is blood.

Additional aspects of the invention provide tools applicable in the methods provided herein. Hence, an aspect provides a set of amplification primer pairs suitable for polymerase-based nucleic acid amplification, comprising an amplification primer pair configured to detect the presence or absence of exon 7 of the DMD gene, an amplification primer pair configured to detect the presence or absence of exon 43 of the DMD gene, an amplification primer pair configured to detect the presence or absence of exon 44 of the DMD gene, an amplification primer pair configured to detect the presence or absence of exon 45 of the DMD gene, an amplification primer pair configured to detect the presence or absence of exon 46 of the DMD gene, an amplification primer pair configured to detect the presence or absence of exon 49 of the DMD gene, an amplification primer pair configured to detect the presence or absence of exon 50 of the DMD gene, an amplification primer pair configured to detect the presence or absence of exon 51 of the DMD gene, an amplification primer pair configured to detect the presence or absence of exon 52 of the DMD gene, and an amplification primer pair configured to detect the presence or absence of exon 54 of the DMD gene. Each individual primer and primer pair as discussed throughout this specification is also separately disclosed.

Another aspect provides a set of oligonucleotide probes configured to hybridise with the target nucleic acid regions as taught herein. Each individual oligonucleotide probe as discussed throughout this specification is also separately disclosed.

A further aspect provides a composition comprising the set of amplification primer pairs and/or the set of oligonucleotide probes as taught herein.

Another aspect provides a kit of parts comprising the set of amplification primer pairs and/or the set of oligonucleotide probes as taught herein, and optionally further comprising reagents sufficient for formulating a polymerase-based nucleic acid amplification reaction mixture.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates amplification curves and scattered endpoint fluorescence of control group and carrier group, and DMD patients with a deletion of at least one target exon (deleted group). 1A. Amplification curves of a patient without deletion of any target exons. 1B. Amplification curves of a patient with a deletion of exons 50 to 54. 1C. Endpoint fluorescence (dots) of control and carrier group versus deleted group (i.e. DMD patients with a deletion overlapping at least one of the 5 target exons). RFU, relative fluorescence units.

FIG. 2 illustrates an exon-skipping treatment approach for the deletion of DMD exon 50.

FIG. 3 illustrates endpoint fluorescence of respective target exons for carrier group and DMD patients.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

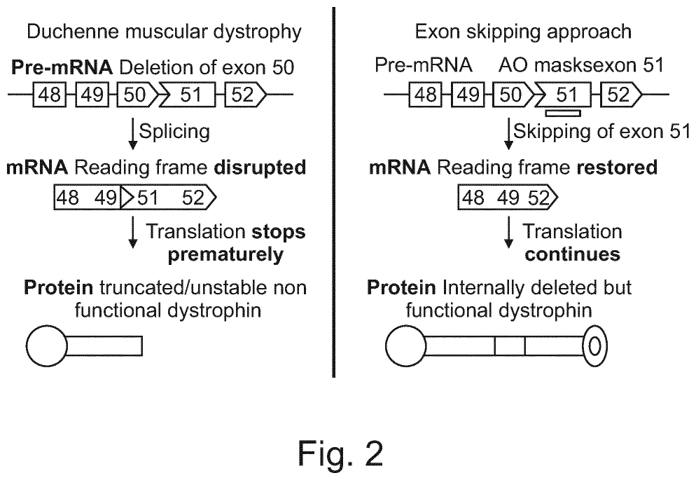

As corroborated by the experimental section, which illustrates certain representative embodiments of the present invention, the inventors provide highly sensitive and specific assays for the genetic analysis of the DMD gene. Genetic alterations in this gene underlie Duchenne Muscular Dystrophy (DMD), an X-linked devastating muscle disease with an early onset. The present assays allow to detect the presence or absence, such as a deletion or partial deletion, of at least exons 44, 46, 50, 52 and 54 and/or at least exons 7, 43, 45, 49 and 51, preferably at least exons 7, 43, 44, 45, 46, 49, 50, 51, 52 and 54 of the DMD gene. Deletions of one or more of these exons can be amenable to currently available exon skipping therapies, in particular exon-51, exon-45, exon-53,exon-8, exon-44, or exon-50 skipping treatments, and hence the present methods provide highly valuable information on the potential occurrence of such treatable DMD mutations (DMD patients eligible for either exon-51, exon-45 or exon-53, exon-8, exon-44, or exon-50 skipping treatments account for approximately 30% of all DMD cases). Indeed, there is rising awareness that DMD patients amenable to exon-skipping should be treated as early as possible (i.e., from birth) in order to maximise the beneficial therapeutic effect. A trial is also ongoing to study the safety and efficacy of eteplirsen in children as young as six months (ClinicalTrials identifier: NCT03218995). The present assays thus offer a reliable and robust test that may be used to detect DMD patients carrying deletion(s) of one or more of the aforementioned DMD exons even in new-borns, facilitating very early initiation of the required treatment (currently, due to a delay in clinical diagnosis of 1.3-2.5 years after the appearance of first symptoms, the mean age at diagnosis of DMD patients is 4.43 years, which reduces the chance of an early and successful therapeutic intervention). Additionally, pre-treating patients before gene therapy using an exon-skipping approach could present major benefits as pre-treatment by exon-skipping therapy potentiates the effect of gene therapy. Such pre-treatment would allow the use of lower—and therefore safer—doses of vector to bring about a higher level of dystrophin expression in the long term. The assays may further optionally detect one or more additional genetic alterations in the DMD gene, in particular one or more pathological mutations in the DMD gene. In certain embodiments, the methods detect the presence or absence of at least exons that are selected such that a deletion in the DMD gene spanning one or more of the exons shifts the reading frame of the DMD gene downstream of the deletion and the reading frame can be restored by exon-skipping therapy. Exon-skipping approaches using antisense oligonucleotides (AO) ‘masking’ a particular DMD exon such that said exon is omitted from the DMD transcript are conceptually well-understood. A schematic illustration of exon-skipping of DMD exon 51 to restore a reading frame shift caused by the deletion of DMD exon 50 is shown in FIG. 2. Additionally, multi-exon-skipping approaches in which all DMD exons from exon 45 to exon 55 are skipped are also known. Such approaches can potentially address any mutation or deletion in any of these exons which alters the reading frame (such mutations are found in as many as 63% of DMD patients). A DMD transcript in which exon 44 is linked to exon 56 has restored reading frame, and produces internally deleted (Δ45-55) dystrophin, which is known to be at least partly functional from BMD patients.

Accordingly, an aspect of the invention provides a method for analysing the Duchenne Muscular Dystrophy (DMD) gene in a sample containing genetic material of a subject, wherein the method detects the presence or absence of at least exons 44, 46, 50, 52 and 54 and/or exons 7, 43, 45, 49 and 51 of the DMD gene, preferably at least exons 7, 43, 44, 45, 46, 49, 50, 51, 52 and 54 of the DMD gene in the genetic material of the subject, wherein the detection of the presence or absence of the exons comprises multiplex polymerase-based nucleic acid amplification. The design of informative and dependable polymerase-based nucleic acid amplifications in a molecular diagnosis context remains a challenging task in view of the multitude of factors which may impact on such reactions. The complexity becomes immensely greater when multiplexed amplifications of more than one loci or target sequences are intended. The present inventors have successfully tackled these complexities.

Another aspect of the invention provides a method for analysing the DMD gene in a sample containing genetic material of a subject, wherein the method detects the presence or absence of at least exons 44, 46, 50, 52 and 54 and/or exons 7, 43, 45, 49 and 51 of the DMD gene, preferably at least exons 7, 43, 44, 45, 46, 49, 50, 51, 52 and 54 of the DMD gene in the genetic material of the subject, and wherein the sample is blood.

Duchenne muscular dystrophy is caused by mutations in the gene encoding dystrophin (DMD gene), a large protein containing an N-terminal actin-binding domain and multiple spectrin repeats. Dystrophin forms a component of the dystrophin-glycoprotein complex (DGC) which bridges the inner cytoskeleton and the extracellular matrix. Alternative promoter usage and alternative splicing result in numerous distinct transcript variants and protein isoforms for the DMD gene. Deletions, duplications, and point mutations at the DMD gene locus may cause the severe Duchenne muscular dystrophy (DMD), the milder Becker muscular dystrophy (BMD), or cardiomyopathy. Approximately two-thirds of the mutations in both DMD and BMD are deletions of one or more exons in the dystrophin gene. Although there is no clear correlation found between the extent of the deletion and the severity of the disorder, DMD deletions usually result in frameshift.

The human DMD (dystrophin) gene, which spans a genomic range of greater than 2 Mb on the X chromosome, is annotated under U.S. government's National Center for Biotechnology Information (NCBI) Genbank (http://www.ncbi.nlm.nih.gov/) Gene ID no. 1756.

A reference genomic sequence of the human DMD gene is annotated under Genbank accession no: NG_012232.1. Various human DMD transcript isoforms and corresponding dystrophin protein sequences are also annotated in the Genbank inter alia under accession no: NM_000109.4/NP_000100.3 (isoform Dp427c), NM_004006.3/NP_003997.2 (isoform Dp427m), NM_004009.3/NP_004000.1 (isoform Dp427p1), NM_004010.3/NP_004001.1 (isoform Dp427p2), NM_004011.4/NP_004002.3 (isoform Dp260-1), NM_004012.4/NP_004003.2 (isoform Dp260-2), NM_004013.2/NP_004004.1 (isoform Dp140), NM_004014.2/NP_004005.1 (isoform Dp116), NM_004015.3/NP_004006.1 (isoform Dp71), NM_004016.3/NP_004007.1 (isoform Dp71b), NM_004017.3/NP_004008.1 (isoform Dp71a), NM_004018.3/NP_004009.1 (isoform Dp71ab), etc.

Further, a representative sequence of exon 7 of the human DMD gene is set forth below in SEQ ID NO: 66 with the exon 7 (coding) sequence shown in upper case and the surrounding intronic sequences shown in lower case:

(SEQ ID NO: 66)
atgtgtgtatgtgtatgtgttttagGCCAGACCTATTTGACTGGAATAGT
GTGGTTTGCCAGCAGTCAGCCACACAACGACTGGAACATGCATTCAACAT
CGCCAGATATCAATTAGGCATAGAGAAACTACTCGATCCTGAAGgttggt
aaatttctggactaccact

Further, a representative sequence of exon 43 of the human DMD gene is set forth below in SEQ ID NO: 67 with the exon 43 (coding) sequence shown in upper case and the surrounding intronic sequences shown in lower case:

(SEQ ID NO: 67)
ctgttttaaaatttttatattacagAATATAAAAGATAGTCTACAACAAA
GCTCAGGTCGGATTGACATTATTCATAGCAAGAAGACAGCAGCATTGCAA
AGTGCAACGCCTGTGGAAAGGGTGAAGCTACAGGAAGCTCTCTCCCAGCT
TGATTTCCAATGGGAAAAAGTTAACAAAATGTACAAGGACCGACAAGGgt
aggtaacacatatatttttcttg

Further, a representative sequence of exon 44 of the human DMD gene is set forth below in SEQ ID NO: 31, with the exon 44 (coding) sequence shown in upper case and the surrounding intronic sequences shown in lower case:

(SEQ ID NO: 31)
aaaaattgcaaccttccatttaaaatcagcttttatattgagtatttttt
taaaatgttgtgtgtacatgctaggtgtgtatattaatttttatttgtta
cttgaaactaaactctgcaaatgcaggaaactatcagagtgatatctttg
tcagtataaccaaaaaatatacgctatatctctataatctgttttacata
atccatctatttttcttgatccatatgcttttacctgcagGCGATTTGAC
AGATCTGTTGAGAAATGGCGGCGTTTTCATTATGATATAAAGATATTTAA
TCAGTGGCTAACAGAAGCTGAACAGTTTCTCAGAAAGACACAAATTCCTG
AGAATTGGGAACATGCTAAATACAAATGGTATCTTAAGgtaagtctttga
tttgttttttcgaaattgtatttatcttcagcacatctggactctttaac
ttcttaaagatcaggttctgaagggtgatggaaattacttttgactgttg
ttgtcatcattatattactagaaagaaaattatcataatgataatattag
agcacggtgctatggactttttgtgtcaggatgagagagtttgcctggac
ggagctggtttatctgataaactgcaaa.

Further, a representative sequence of exon 45 of the human DMD gene is set forth below in SEQ ID NO: 68 with the exon 45 (coding) sequence shown in upper case and the surrounding intronic sequences shown in lower case:

(SEQ ID NO: 68)
gttttgcctttttggtatcttacagGAACTCCAGGATGGCATTGGGCAGC
GGCAAACTGTTGTCAGAACATTGAATGCAACTGGGGAAGAAATAATTCAG
CAATCCTCAAAAACAGATGCCAGTATTCTACAGGAAAAATTGGGAAGCCT
GAATCTGCGGTGGCAGGAGGTCTGCAAACAGCTGTCAGACAGAAAAAAGA
Ggtagggcgacagatctaataggaat

A representative sequence of exon 46 of the human DMD gene is set forth below in SEQ ID NO: 32, with the exon 46 (coding) sequence shown in upper case and the surrounding intronic sequences shown in lower case:

(SEQ ID NO: 32)
ggccaggaatttttgaatcagaatttttcttgttcgattttaatctctta
tcatttagagattcttgaaatattgaaattactttgttcaaagtgaatga
attttcttaaattatgtatggttaacatcttttaaattgcttatttttaa
attgccatgtttgtgtcccagtttgcattaacaaatagtttgagaactat
gttggaaaaaaaaataacaattttattcttctttctccagGCTAGAAGAA
CAAAAGAATATCTTGTCAGAATTTCAAAGAGATTTAAATGAATTTGTTTT
ATGGTTGGAGGAAGCAGATAACATTGCTAGTATCCCACTTGAACCTGGAA
AAGAGCAGCAACTAAAAGAAAAGCTTGAGCAAGTCAAGgtaattttattt
tctcaaatcccccagggcctgcttgcataaagaagtatatgaatctattt
tttaattcaatcattggttttctgcccattaggttattcatagttccttg
ctaaagtgtttttctcacaactttatttcttcttaaccctgcagttctga
accagtgcacataagaacatatgtatatatgtgtgtgtgtgtatttatat
atacacacacacatattgcatctataca.

Further, a representative sequence of exon 49 of the human DMD gene is set forth below in SEQ ID NO: 69 with the exon 49 (coding) sequence shown in upper case and the surrounding intronic sequences shown in lower case:

(SEQ ID NO: 69)
gatctgcaatacatgtggagtctccaagggtatatttaaatttagtaatt
ttattgctaactgtgaagttaatctgcactatatgggttcttttccccag
GAAACTGAAATAGCAGTTCAAGCTAAACAACCGGATGTGGAAGAGATTTT
GTCTAAAGGGCAGCATTTGTACAAGGAAAAACCAGCCACTCAGCCAGTGA
AGgtaatgaagcaacctctagcaatatccattacctcataatgggttatg
cttcccctgttg

A representative sequence of exon 50 of the human DMD gene is set forth below in SEQ ID NO: 33, with the exon 50 (coding) sequence shown in upper case and the surrounding intronic sequences shown in lower case:

(SEQ ID NO: 33)
tgtagggtggttggctaaaataattataattcctttaaaagaaattctac
ccactaaagttaatttagaagtaaaatataatagaaatccaataatatat
tcaccaaatggattaagatgttcatgaattatcttcaaagtgttaatcga
ataagtaatgtgtatgcttttctgttaaagAGGAAGTTAGAAGATCTGAG
CTCTGAGTGGAAGGCGGTAAACCGTTTACTTCAAGAGCTGAGGGCAAAGC
AGCCTGACCTAGCTCCTGGACTGACCACTATTGGAGCCTgtaagtatact
ggatcccattctctttggctctagctatttgttcaaaagtgcaactatga
agtgatgactgggtgagagagaaaatttgtttcaattctaaagatagaga
taaacctttgtgttattgactgtgcaaaaagtcttagagtacattccttg
gaaattgactctgattcaa.

Further, a representative sequence of exon 51 of the human DMD gene is set forth below in SEQ ID NO: 70 with the exon 51 (coding) sequence shown in upper case and the surrounding intronic sequences shown in lower case:

(SEQ ID NO: 70)
tttgcaaaaacccaaaatattttagCTCCTACTCAGACTGTTACTCTGGT
GACACAACCTGTGGTTACTAAGGAAACTGCCATCTCCAAACTAGAAATGC
CATCTTCCTTGATGTTGGAGGTACCTGCTCTGGCAGATTTCAACCGGGCT
TGGACAGAACTTACCGACTGGCTTTCTCTGCTTGATCAAGTTATAAAATC
ACAGAGGGTGATGGTGGGTGACCTTGAGGATATCAACGAGATGATCATCA
AGCAGAAGgtatgagaaaaaatgataaaagttg

A representative sequence of exon 52 of the human DMD gene is set forth below in SEQ ID NO: 34, with the exon 52 (coding) sequence shown in upper case and the surrounding intronic sequences shown in lower case:

(SEQ ID NO: 34)
ccatttgagcctttaaatgaagaaaatctatagtcaagattttcatttga
aatatttttgatatctaagaatgaaacatatttcctgttaaattgttttc
tataaacccttatacagtaacatcttttttatttctaaaagtgttttggc
tggtctcacaattgtactttactttgtattatgtaaaaggaatacacaac
gctgaagaaccctgatactaagggatatttgttcttacagGCAACAATGC
AGGATTTGGAACAGAGGCGTCCCCAGTTGGAAGAACTCATTACCGCTGCC
CAAAATTTGAAAAACAAGACCAGCAATCAAGAGGCTAGAACAATCATTAC
GGATCGAAgtaagttttttaacaagcatgggacacacaaagcaagatgca
tgacaagtttcaataaaaacttaagttcatatatccccctcacatttata
aaaataatgtgaaataattgtaaatgataacaattgtgctgagattttca
gtccataatgttaccttttaataaatgaatgtaattccattgaatagaag
aaatacatttttaaatcaattcagggcttatatagttgcaaagcatgc.

A representative sequence of exon 54 of the human DMD gene is set forth below in SEQ ID NO: 35, with the exon 54 (coding) sequence shown in upper case and the surrounding intronic sequences shown in lower case:

(SEQ ID NO: 35)
aaaggtgggttaccttatactgtcatgattgactaaatcatatggtaggt
taaaagcaatctaatatatgtattctgacctgaggattcagaagctgttt
acgaagtattttaagacactccaactagagatttcataaaaaaaactgac
attcattctctttctcataaaaatctatagCAGTTGGCCAAAGACCTCCG
CCAGTGGCAGACAAATGTAGATGTGGCAAATGACTTGGCCCTGAAACTTC
TCCGGGATTATTCTGCAGATGATACCAGAAAAGTCCACATGATAACAGAG
AATATCAATGCCTCTTGGAGAAGCATTCATAAAAGgtatgaattacatta
tttctaaaactactgttggctgtaataatggggtggtgaaactggatgga
ccatgaggatttgtttttccaatccagctaaactggagcttgggagggtt
caagacgataaataccaactaaactcacggacttggctcagacttctatt
ttaaaaacgaggaac.

The dystrophin gene and protein have orthologues in many other species, and many dystrophin gene and protein sequences have been publically annotated (e.g., Genbank lists 235 DMD orthologues from vertebrate species). By means of an illustration and without limitation, a dog (Canis lupus familiaris) dystrophin gene is annotated under Genbank Gene ID no. 606758 (genomic reference sequence NC_006621.3), a mouse (Mus musculus) dystrophin gene is annotated under Genbank Gene ID no. 13405 (genomic reference sequence NC_000086.7), a chimpanzee (Pan troglodytes) dystrophin gene is annotated under Genbank Gene ID no. 465559 (genomic reference sequence NC_036902.1), a pig (Sus scrofa) dystrophin gene is annotated under Genbank Gene ID no. 497636 (genomic reference sequence NC_010461.5), a cattle (Bos taurus) dystrophin gene is annotated under Genbank Gene ID no. 537655 (genomic reference sequence NC_037357.1), a horse (Equus caballus) dystrophin gene is annotated under Genbank Gene ID no. 100051515 (genomic reference sequence NC_009175.3), etc.

The term “sample” as conventionally understood refers to a limited quantity, piece or specimen that shows the quality (i.e., is representative or characteristic of the properties) of the whole (e.g., an object or material) from which it was removed or taken. In particular, the terms “sample” or “biological sample” as used throughout this specification may denote a biological specimen obtained (isolated, removed) from a subject. Samples may include without limitation organ tissue, whole blood, a blood fraction, plasma, serum, whole blood cells, white blood cells (e.g., peripheral blood mononuclear cells), saliva, urine, stool, tears, sweat, sebum, lymph, amniotic fluid, cell lysates, etc. Preferably, a sample may be readily obtainable by non-invasive or minimally invasive methods, such as blood collection, urine collection, stool collection, tissue biopsy, etc. allowing the provision/removal/isolation of the sample from a subject. The tissue may be from a living subject or may be cadaveric tissue, preferably may be from a living subject. Any suitable weight or volume of a sample may be removed from a subject for analysis. Without limitation, a liquid sample may have a volume between 10 μl and 20 ml. A solid sample may have a weight of between 10 μg and 20 g.

A sample as intended herein contains genetic material of a subject. Hence, particularly useful samples are those known to comprise or expected or predicted to comprise genetic material of the subject. Genetic material in the present context encompasses any nucleic acid molecule or molecules in which the structure or sequence of the subject's DMD gene can be evaluated, and may particularly encompass subject's nuclear deoxyribonucleic acid (DNA), i.e., subject's nuclear genomic DNA.

In certain embodiments, the present methods and tools may be directly applied to the sample. In other embodiments, the present methods and tools may be applied to nucleic acids isolated from the sample. Nucleic acids, such as DNA, particularly genomic DNA, can be extracted or isolated from DNA-containing samples in ways known in the art. The terms “extracting” or “isolating” with reference to a particular component (such as DNA) of a composition or mixture (such as a sample) encompasses processes or techniques whereby such component is separated from one or more or (substantially) all other components of the composition or mixture. The term does not require absolute purity. Instead, isolating the component will produce a discrete environment in which the abundance of the component relative to one or more or all other components is greater than in the starting composition or mixture. A discrete environment may denote a single medium, such as for example a single solution, dispersion, gel, precipitate, etc. Quantity of nucleic acids may be determined by measuring absorbance A260. Purity of nucleic acids may be determined by measuring absorbance A260/A280, or by agarose- or polyacrylamide-gel electrophoresis and ethidium bromide or similar staining. Conventional techniques for extracting or isolating DNA, particularly genomic DNA, include without limitation organic (phenol-chloroform) extraction, non-organic (proteinase K and salting-out) extraction, ion exchange resin extraction, or silica exchange resin extraction.

In certain preferred embodiments, the sample comprises, consists essentially of or consists of blood. Particularly preferably, the sample is blood. Whole blood may typically be employed, even while the use of any fraction of blood containing nuclear genomic DNA of the subject, such as buffy coat or isolated leukocytes, is also contemplated. In certain embodiments, the sample may be fresh unclotted whole blood (preferably with EDTA as anticoagulant). Such samples can be conveniently kept at room temperature for up to about 72 hours before DNA isolation and screening. Typically, whole blood samples may be about 5 ml for infants and adults, and about 1 ml for newborns. In certain embodiments, the sample may be dried blood. A convenient way of preparing and handling dried blood samples is to apply a small volume of whole blood, such as a drop or a few drops of blood typically drawn by lancet from the finger, heel or toe, onto absorbent filter paper, air dry the blood spot(s) for several hours, and store the specimen in low gas-permeability plastic bags with desiccant added to reduce humidity at ambient temperature. In a laboratory, disc of blood-saturated paper can be punched out from the specimen, and blood can be eluted out in phosphate buffered saline containing 0.05% Tween 80 and 0.005% sodium azide overnight at 4° C. Hence, in certain embodiments, the sample is whole blood or any fraction of blood containing DNA, or dried blood, more preferably a dried blood spot.

The instant methods detect the presence or absence of at least exons 44, 46, 50, 52 and 54 and/or exons 7, 43, 45, 49 and 51 of the DMD gene, preferably at least exons 7, 43, 44, 45, 46, 49, 50, 51, 52 and 54 of the DMD gene in the genetic material of the subject, and may optionally be extended or supplemented to detect one or more additional genetic alterations in the DMD gene, in particular one or more pathological mutations in the DMD gene. Any such additional mutation or mutations are contemplated, including deletions, insertions and/or substitutions, for example, missense or non-sense point mutations, duplications, frameshift mutations, exon deletions, etc. in the DMD gene. Such additional DMD mutation or mutations may be evaluated sequentially to (in any order) or simultaneously with the detection of the presence or absence of DMD exons 44, 46, 50, 52 and 54 and/or DMD exons 7, 43, 45, 49 and 51, preferably DMD exons 7, 43, 44, 45, 46, 49, 50, 51, 52 and 54, and in certain embodiments the simultaneous evaluation of such additional DMD mutation or mutations may be multiplexed with the detection of the presence or absence of DMD exons 44, 46, 50, 52 and 54 and/or DMD exons 7, 43, 45, 49 and 51, preferably DMD exons 7, 43, 44, 45, 46, 49, 50, 51, 52 and 54. In certain preferred embodiments, the evaluation of such additional DMD mutation or mutations may involve polymerase-based nucleic acid amplification, especially where this is also the case for the detection of the presence or absence of DMD exons 44, 46, 50, 52 and 54 and/or DMD exons 7, 43, 45, 49 and 51, preferably DMD exons 7, 43, 44, 45, 46, 49, 50, 51, 52 and 54.

The genomic organisation of the dystrophin gene, including the numbering of the individual DMD exons, is well-established. For example, exons 42-79 of the human DMD gene have been mapped as early as 1995 (Nobile et al. Genomics 1995, vol. 28(1), 97-100), and the human DMD genomic structure and all exons are also annotated in the Genbank genomic nucleic acid entry NG_012232.1. By means of additional guidance, representative sequences of human DMD exons 7, 43, 44, 45, 46, 49, 50, 51, 52 and 54 and their respective flanking partial intronic sequences are set forth above in SEQ ID NO: 31-35 and SEQ ID NO: 66-70, respectively. While certain sequences are specifically individualised herein, the skilled person understands that the native nucleic acid sequences of a given locus, gene, exon or intron may differ between or within different individuals of the same species due to normal genetic diversity or variation within such species, and that such naturally-occurring sequence variations or polymorphisms are subsumed by the reference to such genetic elements.

The instant methods may be contemplated to provide a principally binary answer to the query whether the genetic material of a subject contains or does not contain exon 7, 43, 44, 45, 46, 49, 50, 51, 52 and/or 54 of the DMD gene. By means of an example, the methods may thus conclude that any one, or any two, or any three, or any four, or any five, any six, any seven, any eight, any nine, or all ten of DMD exons 7, 43, 44, 45, 46, 49, 50, 51, 52 and 54 are absent from the genetic material of the subject, whereas the remaining ones of DMD exons 7, 43, 44, 45, 46, 49, 50, 51, 52 and 54 are present. For example, the methods may thus conclude that any one, or any two, or any three, or any four or all five of DMD exons 44, 46, 50, 52 and 54 and/or any one, or any two, or any three, or any four or all five of DMD exons 7, 43, 45, 49 and 51 are absent from the genetic material of the subject, whereas the remaining ones of DMD exons 44, 46, 50, 52 and 54 and/or DMD exons 7, 43, 45, 49 and 51 are present. The relevance of this information lies in the fact that a large proportion of pathogenic mutations in patients with DMD or BMD are deletions of one or more DMD exons, including one or more DMD exons selected from DMD exons 7, 43, 44, 45, 46, 49, 50, 51, 52 and 54. Because the dystrophin gene resides at the X chromosome, males only carry one DMD gene allele, i.e., are hemizygous for the DMD gene, and the deletion of one or more exons in that DMD gene allele will mean that such exon or exons will be altogether absent in the genetic material of a male subject. Typically, a deletion in the DMD gene may span more than one exon, such as for example a deletion may span or remove DMD exons 13-44, 20-44, 46-55, 45-55, 46-53, 46-52, 45-50, 45-48, 45-47, 45-46, 46-48, 45-47, 45-48, 48-55, 49-54, 51-55, or 51-64, or so on. Accordingly, where two or more of DMD exons 7, 43, 44, 45, 46, 49, 50, 51, 52 and 54 are absent from the genetic material of the a subject, these will typically be two or more exons that are adjacent or successive in the series 7, 43, 44, 45, 46, 49, 50, 51, 52, 54.

Disease-causing deletions in the DMD gene may commonly eliminate the whole or entire sequence of one or more DMD exons, and in case of the deletion of multiple exons also the intronic sequences interposed there between. Hence, the reference to the absence of an exon may typically contemplate the deletion of an entire exon, i.e., the deletion of the complete exon sequence (typically together with the intronic sequences adjacent to the exon), but the deletion of a portion of the exon is also encompassed. A portion of an exon in this context denotes a substantial portion of the exon, such as the deletion of at least 20% or at least 50% or at least 70% of the exon sequence, rather than minor deletions such as deletions affecting a single one or only a few nucleotides.

The term “subject”, “individual” or “patient” are used interchangeably throughout this specification, and typically and preferably denote humans, but may also encompass reference to non-human animals, preferably warm-blooded animals, more preferably vertebrates, yet more preferably higher animals, still more preferably non-human mammals or non-human primates. Non-limiting examples of such animals include rodents, canines, felines, equines, ovines, or porcines; such as for example pets (e.g., dogs, cats, rabbits, gerbils, hamsters, chinchillas, mice, rats, guinea pigs, donkeys, mules, ferrets, pygmy goats, pot-bellied pigs; avian pets such as canaries, parakeets, parrots, chickens, turkeys; reptile pets, such as lizards, snakes, tortoises and turtles; aquatic pets, such as fish, frogs), experimental animals (e.g., mice, rats, guinea pigs, rabbits, dogs, pigs, monkeys, ferrets, sheep), and livestock animals (e.g., alpaca, banteng, bison, camel, cattle (cows), deer, donkey, gayal, goat, horse, llama, mule, pig, pony, reindeer, sheep, water buffalo, yak). In certain embodiments, the subject may be an experimental animal or animal substitute as a disease model. In certain embodiments, the subject is a mammal. Particularly preferred are human subjects. The term does not denote a particular age or sex. Thus, adult and new-born subjects, as well as foetuses, whether male or female, are intended to be covered. The term subject is further intended to include transgenic non-human species.

Pathologies caused by mutations of the X-linked DMD gene, such as DMD, are expected to affect only male patients, whereas females heterozygous for the disease-causing mutation constitute asymptomatic carriers. Accordingly, in preferred embodiments, the subject may be a male, in particular a human male. However, as the added logistics and cost of sorting and separately handling samples from male and female subjects may exceed the added cost of performing the method for both types of samples and neglecting the results from the female subjects in downstream reviewing and reporting (female samples are not expected to show the absence of the DMD exons in question, since even carrier females will have one intact DMD allele), in certain embodiments the subject may be a female, in particular a human female.

Whereas the term “subject” is as such not restricted to any particular age, DMD is typically a severe and early onset pathology—first symptoms typically start before the age of 3 years. Accordingly, to facilitate early diagnosis and treatment, insofar treatment is available, early screening or testing may be desirable. Accordingly, in certain embodiments, a subject, such as in particular a human subject may be 5 years old or less, such as 4 years old or less, such as preferably 3 years old or less, such as more preferably 2 years old or less, such as even more preferably 1 year old or less, such as for example 12 months, 11 months, 10 months, 9 months, 8 months, 7 months, 6 months, 5 months, 4 months, 3 months, 2 months, or 1 month old or less. In certain particularly preferred embodiments, a subject may be a neonate (new-born), for example a human subject may be within the first 4 weeks or 28 days from birth. Preferably, a human subject may be within the first 5 days, more preferably within the first 48 hours from birth. In certain embodiments, the present methods, which can be run in practical and cost-effective way, can allow to make DMD screening a part of the standard new-born screening (NBS) tests, which typically employ dried blood spot (DBS) material as explained elsewhere in this specification. Advantageously, in many countries DNA is already extracted from DBS material for the new-born screening of spinal muscular atrophy, and such DNA material can also be readily used for the present methods. This fact may further simplify the inclusion of the present methods in the Recommended Uniform Screening Panel (RUSP) in NBS context.

In certain aspects, the present methods and tools may rely on polymerase-based nucleic acid amplification. The phrase “polymerase-based nucleic acid amplification” as used herein generally encompasses any in vitro process for increasing the number of copies of a target nucleic acid region within a nucleic acid molecule, preferably within a DNA molecule, by the action of a nucleic acid polymerase, e.g., DNA polymerase. The process may encompass both linear and exponential amplification, and particularly preferably refers to exponential amplification. The process may particularly preferably refer to polymerase chain reaction (PCR). In PCR, target nucleic acid region within a nucleic acid molecule, especially within a DNA molecule, is amplified using thermostable DNA polymerase(s) and at least two amplification primers, one complementary to the (+)-strand at one end of the target sequence to be amplified and the other complementary to the (−)-strand at the other end of the target sequence. A reference to PCR as used herein encompasses modifications of the prototypic PCR, such as, e.g., high-fidelity PCR, hot-start PCR, touch-down PCR, nested PCR, multiplex PCR, quantitative PCR, quantitative real-time PCR, long-range PCR, RT-PCR, etc. (see, e.g., PCR Protocols: A Guide to Methods and Applications, eds. Innis et al., Academic Press, San Diego, 1990).

Hence, in preferred embodiments, the polymerase-based nucleic acid amplification may be polymerase chain reaction (PCR).

In certain embodiments, the polymerase-based nucleic acid amplification may be quantitative, i.e., it provides information about the quantity of the amplification products and by extension about the quantity of the templates (i.e., DMD gene target sequences). In particularly preferred embodiments, the polymerase-based nucleic acid amplification may be real-time quantitative amplification, more preferably real-time quantitative PCR. Real-time quantitative PCR is commonly known in the art as simply “quantitative PCR” (qPCR, QPCR) or as real-time qPCR, real-time QPCR, RT-qPCR or RT-QPCR. Real-time quantitative PCR may be preferred under some circumstances, because it provides not only a quantitative measurement, but also reduced time and contamination. Hence, as taught herein, in certain embodiments, the polymerase-based nucleic acid amplification is real-time quantitative amplification, preferably real-time quantitative PCR (qPCR).

In certain aspects and particularly preferred embodiments, the polymerase-based nucleic acid amplification as taught herein, such as PCR or QPCR, may be multiplexed, such that at least two, preferably at least three, at least four, more preferably at least five, at least six, at least seven, at least eight, at least nine, and most preferably all ten DMD exons 7, 43, 44, 45, 46, 49, 50, 51, 52 and 54, and optionally one or more additional DMD genetic mutations, such as the deletion of one or more additional DMD exons, are amplified and detected in the same polymerase-based nucleic acid amplification reaction. In certain preferred embodiments, the multiplex polymerase-based nucleic acid amplification is multiplex real-time quantitative amplification, preferably multiplex real-time quantitative PCR (qPCR). Hence, in certain embodiments, the detection of the presence or absence of at least exons 44, 46, 50, 52 and 54 of the DMD gene or the detection of the presence or absence of at least exons 7, 43, 45, 49, and 51 of the DMD gene in the genetic material of the subject is multiplexed in a single polymerase-based nucleic acid amplification reaction. For example, the detection of the presence or absence of at least exons 44, 46, 50, 52 and 54 of the DMD gene may be multiplexed in one single polymerase-based nucleic acid amplification reaction, and the detection of the presence or absence of at least exons 7, 43, 45, 49, and 51 of the DMD gene may be multiplexed in another, separate single polymerase-based nucleic acid amplification reaction, whereby the two separate multiplexed reactions can together yield information on all exons 7, 43, 44, 45, 46, 49, 50, 51, 52 and 54. Preferably, in certain embodiments, the detection of the presence or absence of at least exons 7, 43, 44, 45, 46, 49, 50, 51, 52 and 54 of the DMD gene in the genetic material of the subject is multiplexed in a single polymerase-based nucleic acid amplification reaction. By reducing the number of reactions to set-up, multiplexing advantageously reduces the amount of sample needed, cuts down the processing and machine space required, reduces variability between reactions, etc.

The term “nucleic acid” as used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U), which are widespread in naturally-occurring nucleic acids, other naturally-occurring bases (e.g., xanthine, inosine, hypoxanthine), as well as chemically or biochemically modified (e.g., methylated), non-natural or derivatised bases. Exemplary modified nucleobases include, without limitation, 5-substituted pyrimidines, 6-azapyrimidines and N-2, N-6 and O-6 substituted purines, including 2-aminopropyladenine, 5-propynyluracil and 5-propynylcytosine. In particular, 5-methylcytosine substitutions have been shown to increase nucleic acid duplex stability. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as, without limitation, 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C₁-C₄ linear or branched, saturated or unsaturated hydrocarbon, such as methyl, ethyl, ethenyl, propyl, 1-propenyl, 2-propenyl, and isopropyl.

Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

In the practice of the present invention, the “nucleic acid” to be amplified may particularly preferably refer to deoxyribonucleic acid (DNA), i.e., a polymer composed of deoxyribonucleotides, even more preferably to nuclear genomic DNA.

In certain embodiments, nucleic acids may be isolated from samples and the methods may employ at least a portion of so-isolated nucleic acids. The term “isolated” with reference to a particular component (e.g., a nucleic acid) generally denotes that such component exists in separation from—for example, has been separated from or prepared and/or maintained in separation from—one or more other components of its natural environment. The term “isolated” as used herein may preferably also encompass the qualifier “purified”. The term “purified” with reference to a substance (e.g., a nucleic acid) does not require absolute purity. Instead, it denotes that such substance is in a discrete environment in which its abundance (conveniently expressed in terms of mass or weight or concentration) relative to other relevant substances is greater than in a sample. A discrete environment denotes a single medium, such as for example a single solution, gel, precipitate, lyophilisate, etc. Purified nucleic acids may be obtained by methods routinely known in the art, e.g., nucleic acids released from lysed cells may be precipitated, e.g., by ethanol precipitation, pelleted, washed, and re-suspended in an appropriate buffer. Purity and quantity of nucleic acids may be determined by measuring absorbance A₂₆₀/A₂₈₀.

In certain embodiments, to detect the presence or absence of exon 44 of the DMD gene the polymerase-based nucleic acid amplification may be configured to amplify a target nucleic acid region using an amplification primer pair comprising an amplification primer configured to hybridise within the nucleic acid sequence GATCTGTCAAATCGCCTGCAGGTAAAAGC (SEQ ID NO: 1), preferably within positions 2-28, more preferably within positions 3-27, even more preferably within positions 4-26, still more preferably within positions 5-25, and yet more preferably within positions 6-24 of SEQ ID NO: 1, and an amplification primer configured to hybridise within the nucleic acid sequence TTCTTAAAGATCAGGTTCTGAAGGGTGATGGA (SEQ ID NO: 2), preferably within positions 2-31, more preferably within positions 3-30, even more preferably within positions 4-29, still more preferably within positions 5-28, and yet more preferably within positions 6-29 of SEQ ID NO: 2.

In certain embodiments, which may optionally and preferably be used in combination with the aforementioned embodiments for the detection of exon 44, to detect the presence or absence of exon 46 of the DMD gene the polymerase-based nucleic acid amplification may be configured to amplify a target nucleic acid region using an amplification primer pair comprising an amplification primer configured to hybridise within the nucleic acid sequence TGTTATCTGCTTCCTCCAACCATAAAACAAA (SEQ ID NO: 3), preferably within positions 2-30, more preferably within positions 3-29, even more preferably within positions 4-28, still more preferably within positions 5-27, and yet more preferably within positions 6-26 of SEQ ID NO: 3, and an amplification primer configured to hybridise within the nucleic acid sequence TTCAATCATTGGTTTTCTGCCCATTAGGTT (SEQ ID NO: 4), preferably within positions 2-29, more preferably within positions 3-28, even more preferably within positions 4-27, still more preferably within positions 5-26, and yet more preferably within positions 6-25 of SEQ ID NO: 4.

In certain embodiments, which may optionally and preferably be used in combination with the aforementioned embodiments for the detection of exons 44 and/or 46 (preferably exons 44 and 46), to detect the presence or absence of exon 50 of the DMD gene the polymerase-based nucleic acid amplification may be configured to amplify a target nucleic acid region using an amplification primer pair comprising an amplification primer configured to hybridise within the nucleic acid sequence AAACGGTTTACCGCCTTCCACTCAGAGCTC (SEQ ID NO: 5), preferably within positions 2-29, more preferably within positions 3-28, even more preferably within positions 4-27, still more preferably within positions 5-26, and yet more preferably within positions 6-25 of SEQ ID NO: 5, and an amplification primer configured to hybridise within the nucleic acid sequence AACTATGAAGTGATGACTGGGTGAGAGAGAA (SEQ ID NO: 6), preferably within positions 2-30, more preferably within positions 3-29, even more preferably within positions 4-28, still more preferably within positions 5-27, and yet more preferably within positions 6-26 of SEQ ID NO: 6.

In certain embodiments, which may optionally and preferably be used in combination with the aforementioned embodiments for the detection of exons 44, 46 and/or 50 (preferably exons 44, 46 and 50), to detect the presence or absence of exon 52 of the DMD gene the polymerase-based nucleic acid amplification may be configured to amplify a target nucleic acid region using an amplification primer pair comprising an amplification primer configured to hybridise within the nucleic acid sequence TATCAGGGTTCTTCAGCGTTGTGTATTCCTTT (SEQ ID NO: 7), preferably within positions 2-31, more preferably within positions 3-30, even more preferably within positions 4-29, still more preferably within positions 5-28, and yet more preferably within positions 6-27 of SEQ ID NO: 7, and an amplification primer configured to hybridise within the nucleic acid sequence TTTTTAACAAGCATGGGACACACAAAGCAA (SEQ ID NO: 8), preferably within positions 2-29, more preferably within positions 3-28, even more preferably within positions 4-27, still more preferably within positions 5-26, and yet more preferably within positions 6-25 of SEQ ID NO: 8.

In certain embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene the polymerase-based nucleic acid amplification may be configured to amplify a target nucleic acid region using an amplification primer pair comprising an amplification primer configured to hybridise within the nucleic acid sequence CGGAGGTCTTTGGCCAACTGCTATAGATTTTT (SEQ ID NO: 9), preferably within positions 2-31, more preferably within positions 3-30, even more preferably within positions 4-29, still more preferably within positions 5-28, and yet more preferably within positions 6-27 of SEQ ID NO: 9, and an amplification primer configured to hybridise within the nucleic acid sequence GGTGGTGAAACTGGATGGACCATGAGGATT (SEQ ID NO: 10), preferably within positions 2-29, more preferably within positions 3-28, even more preferably within positions 4-27, still more preferably within positions 5-26, and yet more preferably within positions 6-25 of SEQ ID NO: 10.

In certain embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene the polymerase-based nucleic acid amplification may be configured to amplify a target nucleic acid region using an amplification primer pair comprising an amplification primer configured to hybridise within the nucleic acid sequence AAATAGGTCTGGCCTAAAACACATACACATAC (SEQ ID NO: 36), preferably within positions 2-31, more preferably within positions 3-30, even more preferably within positions 4-29, still more preferably within positions 5-28, and yet more preferably within positions 6-27 of SEQ ID NO: 36, and an amplification primer configured to hybridise within the nucleic acid sequence GATCCTGAAGGTTGGTAAATTTCTGGACTACC (SEQ ID NO: 37), preferably within positions 2-31, more preferably within positions 3-30, even more preferably within positions 4-29, still more preferably within positions 5-28, and yet more preferably within positions 6-27 of SEQ ID NO: 37.

In certain embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene the polymerase-based nucleic acid amplification may be configured to amplify a target nucleic acid region using an amplification primer pair comprising an amplification primer configured to hybridise within the nucleic acid sequence ATAATGTCAATCCGACCTGAGCTTTGTTGT (SEQ ID NO: 38), preferably within positions 2-29, more preferably within positions 3-28, even more preferably within positions 4-27, still more preferably within positions 5-26, and yet more preferably within positions 6-25 of SEQ ID NO: 38, and an amplification primer configured to hybridise within the nucleic acid sequence TGTACAAGGACCGACAAGGGTAGGTAACAC (SEQ ID NO: 39), preferably within positions 2-29, more preferably within positions 3-28, even more preferably within positions 4-27, still more preferably within positions 5-26, and yet more preferably within positions 6-25 of SEQ ID NO: 39.

In certain embodiments, which may optionally and preferably be used in combination with the aforementioned embodiments for the detection of exons 7, 43, 44, 46, 50, 52 and/or 54 (preferably exon 7 and 43, more preferably exons 7, 43, 44, 46, 50, 52 and 54), to detect the presence or absence of exon 45 of the DMD gene the polymerase-based nucleic acid amplification may be configured to amplify a target nucleic acid region using an amplification primer pair comprising an amplification primer configured to hybridise within the nucleic acid sequence CTGGAGTTCCTGTAAGATACCAAAAAGGCAAAAC (SEQ ID NO: 40), preferably within positions 2-33, more preferably within positions 3-32, even more preferably within positions 4-31, still more preferably within positions 5-30, and yet more preferably within positions 6-29 of SEQ ID NO: 40, and an amplification primer configured to hybridise within the nucleic acid sequence CTACAGGAAAAATTGGGAAGCCTGAATCT (SEQ ID NO: 41), preferably within positions 2-28, more preferably within positions 3-27, even more preferably within positions 4-26, still more preferably within positions 5-25, and yet more preferably within positions 6-24 of SEQ ID NO: 41.

In certain embodiments, which may optionally and preferably be used in combination with the aforementioned embodiments for the detection of exons 7, 43, 44, 45, 46, 50, 52 and/or 54 (preferably exon 7n 43 and 45, more preferably exons 7, 43, 44, 45, 46, 50, 52 and 54), to detect the presence or absence of exon 49 of the DMD gene the polymerase-based nucleic acid amplification may be configured to amplify a target nucleic acid region using an amplification primer pair comprising an amplification primer configured to hybridise within the nucleic acid sequence TGCTATTTCAGTTTCCTGGGGAAAAGAACC (SEQ ID NO: 42), preferably within positions 2-29, more preferably within positions 3-28, even more preferably within positions 4-27, still more preferably within positions 5-26, and yet more preferably within positions 6-25 of SEQ ID NO: 42, and an amplification primer configured to hybridise within the nucleic acid sequence TCTAGCAATATCCATTACCTCATAATGGGTTATG (SEQ ID NO: 43), preferably within positions 2-33, more preferably within positions 3-32, even more preferably within positions 4-31, still more preferably within positions 5-30, and yet more preferably within positions 6-29 of SEQ ID NO: 43.

In certain embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 45, 46, 49, 50, 52 and 54), to detect the presence or absence of exon 51 of the DMD gene the polymerase-based nucleic acid amplification may be configured to amplify a target nucleic acid region using an amplification primer pair comprising an amplification primer configured to hybridise within the nucleic acid sequence CCACAGGTTGTGTCACCAGAGTAACAGTCTGAGT (SEQ ID NO: 44), preferably within positions 2-33, more preferably within positions 3-32, even more preferably within positions 4-31, still more preferably within positions 5-30, and yet more preferably within positions 6-29 of SEQ ID NO: 44, and an amplification primer configured to hybridise within the nucleic acid sequence TTATAAAATCACAGAGGGTGATGGTGGGTGA (SEQ ID NO: 45), preferably within positions 2-30, more preferably within positions 3-29, even more preferably within positions 4-28, still more preferably within positions 5-27, and yet more preferably within positions 6-26 of SEQ ID NO: 45.

As used herein, the term “primer” or “amplification primer” refers to a single-stranded oligonucleotide, more preferably to a DNA oligonucleotide, which is (or part of which is) complementary or sufficiently complementary to a sequence comprised in a nucleic acid to be amplified by polymerase-based amplification process, e.g., PCR, such that the primer can hybridise (anneal) with said sequence and can act as a point of initiation of synthesis of a primer extension product in the presence of nucleotides and a nucleic acid polymerase, e.g., DNA polymerase. A primer needs to be sufficiently long to prime the synthesis of an extension product. A typical primer may thus be at least 10 nucleotides in length, e.g., at least 11, at least 12, at least 13 or at least 14 nucleotides in length, preferably at least 15 nucleotides in length, e.g., at least 16, at least 17, at least 18 or at least 19 nucleotides in length, more preferably at least 20 nucleotides in length. Further preferred primers are between about 10 and about 40 nucleotides in length, more preferably between about 15 and about 30 nucleotides in length, most preferably between about 18 and about 26 nucleotides long or between about 18 and about 22 nucleotides long, such as particularly preferably 18, 19, 20, 21 or 22 nucleotides long.

The term “oligonucleotide” as used herein refers to a nucleic acid (including nucleic acid analogues and mimetics) oligomer or polymer as defined herein. Preferably, an oligonucleotide is (substantially) single-stranded. Oligonucleotides as intended herein may be preferably between about 10 and about 100 nucleoside units (i.e., nucleotides or nucleotide analogues) in length, preferably between about 15 and about 50, more preferably between about 15 and about 40, also preferably between about 20 and about 30.

The term “primer pair” or “amplification primer pair” refers to a combination of two primers which are suited for amplification of a target nucleic acid region (amplicon) from within a nucleic acid of interest by a polymerase-based amplification process, e.g., PCR. The ability to amplify an amplicon from within the nucleic acid of interest using a primer pair designed to specifically hybridise within the nucleic acid indicates the presence (and optionally quantity) of the nucleic acid in the polymerase-based amplification reaction.

In certain embodiments, primers as taught herein may be defined as configured to hybridise (anneal) within certain recited nucleic acid sequences. In this context, the phrase “hybridise within a nucleic acid” or “hybridise within a nucleic acid sequence” is intended to mean that the primer may anneal to the whole of the recited nucleic acid sequence, or only to a portion of the recited nucleic acid sequence, but does not anneal to sequences adjacent to but outside of the recited nucleic acid sequence.

The terms “hybridisation” and “hybridise” refer to a process by which a nucleic acid strand anneals with complementary or sufficiently complementary sequence(s) comprised in the same or another nucleic acid strand through base pairing, particularly Watson-Crick base pairing. The terms “complementary” or “complementarity” as used herein with reference to nucleic acids, refer to the normal binding of single-stranded nucleic acids under permissive salt (ionic strength) and temperature conditions by base pairing, particularly Watson-Crick base pairing. By means of example, complementary Watson-Crick base pairing occurs between the bases A and T, A and U or G and C. For example, the sequence 5′-A-G-T-3′ is complementary to sequence 5′-A-C-T-3′.

A primer said to hybridise within a given nucleic acid may in certain embodiments be wholly complementary to the sequence or portion thereof with which it anneals. In other embodiments, the primer may be partly but not wholly complementary to said sequence. For example, the primer may display one or more, typically only one or two, substitutions, deletions or additions vis-à-vis a primer that would be wholly complementary to said sequence. Hence, such primer, while not being wholly complementary, is sufficiently complementary to act as a point of initiation of synthesis of a primer extension product in the polymerase-based amplification reaction.

Hybridisation and the strength of hybridisation (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

A primer as taught herein thus comprises an oligonucleotide sequence which effects the hybridisation (annealing) of the primer with its respective target nucleic acid. In certain embodiments, a primer does not contain any further oligonucleotide sequence(s). In certain other embodiments, a primer may contain—besides the oligonucleotide sequence which effects the hybridisation of the primer with its target nucleic acid—additional oligonucleotide sequence(s) serving other useful purpose(s). For example but without limitation, such additional oligonucleotide sequence(s) may provide primer-binding sequences allowing for subsequent amplification or sequencing of the initial amplification product, or may provide probe-binding sequences allowing for subsequent hybridisation of the initial amplification product with probes or capture probes (e.g., on (micro)arrays), or may provide cloning adaptor sequences facilitating cloning of the initial amplification product to nucleic acid constructs, e.g., by restriction enzyme- or recombination-mediated cloning, or may provide linker sequences allowing to couple a primer with another moiety or moieties, e.g., label(s), etc.; various options are available to a skilled reader. Such additional oligonucleotide sequence(s) may be suitably arranged at the 5′ terminus of the primer, such that the primer extension reaction from the 3′ end of the primer is not altered.

In certain embodiments, to detect the presence or absence of exon 44 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18 or preferably 19 contiguous nucleotides, of the nucleic acid sequence TACCTGCAGGCGATTTGAC (SEQ ID NO: 11) or of a sequence diverging from SEQ ID NO: 11 by addition, deletion or substitution of one or two nucleotides, preferably of one nucleotide, or
   comprising, consisting essentially of or consisting of SEQ ID NO: 11; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence CACCCTTCAGAACCTGATCTTT (SEQ ID NO: 12) or of a sequence diverging from SEQ ID NO: 12 by addition, deletion or substitution of one or two nucleotides, preferably of one nucleotide, or
   comprising, consisting essentially of or consisting of SEQ ID NO: 12.

In certain embodiments, which may optionally and preferably be used in combination with the aforementioned embodiments for the detection of exon 44, to detect the presence or absence of exon 46 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20 or preferably 21 contiguous nucleotides, of the nucleic acid sequence TTTATGGTTGGAGGAAGCAGA (SEQ ID NO: 13) or of a sequence diverging from SEQ ID NO: 13 by addition, deletion or substitution of one or two nucleotides, preferably of one nucleotide, or
   comprising, consisting essentially of or consisting of SEQ ID NO: 13; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence AATGGGCAGAAAACCAATGA (SEQ ID NO: 14) or of a sequence diverging from SEQ ID NO: 14 by addition, deletion or substitution of one or two nucleotides, preferably of one nucleotide, or
   comprising, consisting essentially of or consisting of SEQ ID NO: 14.

In certain embodiments, which may optionally and preferably be used in combination with the aforementioned embodiments for the detection of exons 44 and/or 46 (preferably exons 44 and 46), to detect the presence or absence of exon 50 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence CTGAGTGGAAGGCGGTAAAC (SEQ ID NO: 15) or of a sequence diverging from SEQ ID NO: 15 by addition, deletion or substitution of one or two nucleotides, preferably of one nucleotide, or
   comprising, consisting essentially of or consisting of SEQ ID NO: 15; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20 or preferably 21 contiguous nucleotides, of the nucleic acid sequence TCTCACCCAGTCATCACTTCA (SEQ ID NO: 16) or of a sequence diverging from SEQ ID NO: 16 by addition, deletion or substitution of one or two nucleotides, preferably of one nucleotide, or
   comprising, consisting essentially of or consisting of SEQ ID NO: 16.

In certain embodiments, which may optionally and preferably be used in combination with the aforementioned embodiments for the detection of exons 44, 46 and/or 50 (preferably exons 44, 46 and 50), to detect the presence or absence of exon 52 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence AATACACAACGCTGAAGAACCC (SEQ ID NO: 17) or of a sequence diverging from SEQ ID NO: 17 by addition, deletion or substitution of one or two nucleotides, preferably of one nucleotide, or
   comprising, consisting essentially of or consisting of SEQ ID NO: 17; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence TTGTGTGTCCCATGCTTGTT (SEQ ID NO: 18) or of a sequence diverging from SEQ ID NO: 18 by addition, deletion or substitution of one or two nucleotides, preferably of one nucleotide, or
   comprising, consisting essentially of or consisting of SEQ ID NO: 18.

In certain embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence TCTATAGCAGTTGGCCAAAGAC (SEQ ID NO: 19) or of a sequence diverging from SEQ ID NO: 19 by addition, deletion or substitution of one or two nucleotides, preferably of one nucleotide, or
   comprising, consisting essentially of or consisting of SEQ ID NO: 19; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence TCATGGTCCATCCAGTTTCA (SEQ ID NO: 20) or of a sequence diverging from SEQ ID NO: 20 by addition, deletion or substitution of one or two nucleotides, preferably of one nucleotide, or
   comprising, consisting essentially of or consisting of SEQ ID NO: 20.

In certain embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence TGTATGTGTTTTAGGCCAGACC (SEQ ID NO: 46) or of a sequence diverging from SEQ ID NO: 46 by addition, deletion or substitution of one or two nucleotides, preferably of one nucleotide, or
   comprising, consisting essentially of or consisting of SEQ ID NO: 46; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence TCCAGAAATTTACCAACCTTCA (SEQ ID NO: 47) or of a sequence diverging from SEQ ID NO: 47 by addition, deletion or substitution of one or two nucleotides, preferably of one nucleotide, or
   comprising, consisting essentially of or consisting of SEQ ID NO: 47.

In certain embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, or preferably 20 contiguous nucleotides, of the nucleic acid sequence AAAGCTCAGGTCGGATTGAC (SEQ ID NO: 48) or of a sequence diverging from SEQ ID NO: 48 by addition, deletion or substitution of one or two nucleotides, preferably of one nucleotide, or
   comprising, consisting essentially of or consisting of SEQ ID NO: 48; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence ACCTACCCTTGTCGGTCCTT (SEQ ID NO: 49) or of a sequence diverging from SEQ ID NO: 49 by addition, deletion or substitution of one or two nucleotides, preferably of one nucleotide, or
   comprising, consisting essentially of or consisting of SEQ ID NO: 49.

In certain embodiments, which may optionally and preferably be used in combination with the aforementioned embodiments for the detection of exons 7, 43, 44, 46, 50, 52 and/or 54 (preferably exon 7 and 43, more preferably exons 7, 43, 44, 46, 50, 52 and 54), to detect the presence or absence of exon 45 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22 or 23 or preferably 24 contiguous nucleotides, of the nucleic acid sequence GCCTTTTTGGTATCTTACAGGAAC (SEQ ID NO: 50) or of a sequence diverging from SEQ ID NO: 48 by addition, deletion or substitution of one or two nucleotides, preferably of one nucleotide, or
   comprising, consisting essentially of or consisting of SEQ ID NO: 50; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, or preferably 19 contiguous nucleotides, of the nucleic acid sequence CAGGCTTCCCAATTTTTCC (SEQ ID NO: 51) or of a sequence diverging from SEQ ID NO: 51 by addition, deletion or substitution of one or two nucleotides, preferably of one nucleotide, or
   comprising, consisting essentially of or consisting of SEQ ID NO: 51.

In certain embodiments, which may optionally and preferably be used in combination with the aforementioned embodiments for the detection of exons 7, 43, 44, 45, 46, 50, 52 and/or 54 (preferably exons 7, 43 and 45, more preferably exons 7, 43, 44, 45, 46, 50, 52 and 54), to detect the presence or absence of exon 49 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, or preferably 20 contiguous nucleotides, of the nucleic acid sequence TTTTCCCCAGGAAACTGAAA (SEQ ID NO: 52) or of a sequence diverging from SEQ ID NO: 52 by addition, deletion or substitution of one or two nucleotides, preferably of one nucleotide, or
   comprising, consisting essentially of or consisting of SEQ ID NO: 52; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22, 23 or preferably 24 contiguous nucleotides, of the nucleic acid sequence CCCATTATGAGGTAATGGATATTG (SEQ ID NO: 53) or of a sequence diverging from SEQ ID NO: 53 by addition, deletion or substitution of one or two nucleotides, preferably of one nucleotide, or
   comprising, consisting essentially of or consisting of SEQ ID NO: 53.

In certain embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 45, 46, 49, 50, 52 and 54), to detect the presence or absence of exon 51 of the DMD gene the polymerase-based nucleic acid amplification may be configured to amplify a target nucleic acid region using an amplification primer pair comprising:
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22, 23 or preferably 24 contiguous nucleotides, of the nucleic acid sequence GACTGTTACTCTGGTGACACAACC (SEQ ID NO: 54) or of a sequence diverging from SEQ ID NO: 54 by addition, deletion or substitution of one or two nucleotides, preferably of one nucleotide, or
   comprising, consisting essentially of or consisting of SEQ ID NO: 54; and
an amplification primer:
   comprising, consisting essentially of or consisting of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20 or preferably 21 contiguous nucleotides, of the nucleic acid sequence CACCATCACCCTCTGTGATTT (SEQ ID NO: 55) or of a sequence diverging from SEQ ID NO: 55 by addition, deletion or substitution of one or two nucleotides, preferably of one nucleotide, or
   comprising, consisting essentially of or consisting of SEQ ID NO: 55.

In certain preferred embodiments, to detect the presence or absence of exon 44 of the DMD gene the polymerase-based nucleic acid amplification may be configured to amplify a target nucleic acid region using an amplification primer pair comprising an amplification primer of nucleic acid sequence as set forth in SEQ ID NO: 11 and an amplification primer of nucleic acid sequence as set forth in SEQ ID NO: 12.

In certain preferred embodiments, which may optionally and preferably be used in combination with the aforementioned embodiments for the detection of exon 44, to detect the presence or absence of exon 46 of the DMD gene the polymerase-based nucleic acid amplification may be configured to amplify a target nucleic acid region using an amplification primer pair comprising an amplification primer of nucleic acid sequence as set forth in SEQ ID NO: 13 and an amplification primer of nucleic acid sequence as set forth in SEQ ID NO: 14.

In certain preferred embodiments, which may optionally and preferably be used in combination with the aforementioned embodiments for the detection of exons 44 and/or 46 (preferably exons 44 and 46), to detect the presence or absence of exon 50 of the DMD gene the polymerase-based nucleic acid amplification may be configured to amplify a target nucleic acid region using an amplification primer pair comprising an amplification primer of nucleic acid sequence as set forth in SEQ ID NO: 15 and an amplification primer of nucleic acid sequence as set forth in SEQ ID NO: 16.

In certain preferred embodiments, which may optionally and preferably be used in combination with the aforementioned embodiments for the detection of exons 44, 46 and/or 50 (preferably exons 44, 46 and 50), to detect the presence or absence of exon 52 of the DMD gene the polymerase-based nucleic acid amplification may be configured to amplify a target nucleic acid region using an amplification primer pair comprising an amplification primer of nucleic acid sequence as set forth in SEQ ID NO: 17 and an amplification primer of nucleic acid sequence as set forth in SEQ ID NO: 18.

In certain preferred embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene the polymerase-based nucleic acid amplification may be configured to amplify a target nucleic acid region using an amplification primer pair comprising an amplification primer of nucleic acid sequence as set forth in SEQ ID NO: 19 and an amplification primer of nucleic acid sequence as set forth in SEQ ID NO: 20.

In certain preferred embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene the polymerase-based nucleic acid amplification may be configured to amplify a target nucleic acid region using an amplification primer pair comprising an amplification primer of nucleic acid sequence as set forth in SEQ ID NO: 46 and an amplification primer of nucleic acid sequence as set forth in SEQ ID NO: 47.

In certain preferred embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene the polymerase-based nucleic acid amplification may be configured to amplify a target nucleic acid region using an amplification primer pair comprising an amplification primer of nucleic acid sequence as set forth in SEQ ID NO: 48 and an amplification primer of nucleic acid sequence as set forth in SEQ ID NO: 49.

In certain preferred embodiments, which may optionally and preferably be used in combination with the aforementioned embodiments for the detection of exons 7, 43, 44, 46, 50, 52 and/or 54 (preferably exons 7 and 43, more preferably exons 7, 43, 44, 46, 50, 52 and 54), to detect the presence or absence of exon 45 of the DMD gene the polymerase-based nucleic acid amplification may be configured to amplify a target nucleic acid region using an amplification primer pair comprising an amplification primer of nucleic acid sequence as set forth in SEQ ID NO: 50 and an amplification primer of nucleic acid sequence as set forth in SEQ ID NO: 51.

In certain preferred embodiments, which may optionally and preferably be used in combination with the aforementioned embodiments for the detection of exons 7, 43, 44, 45, 46, 50, 52 and/or 54 (preferably exons 7, 43 and 45, more preferably exons 7, 43, 44, 45, 46, 50, 52 and 54), to detect the presence or absence of exon 49 of the DMD gene the polymerase-based nucleic acid amplification may be configured to amplify a target nucleic acid region using an amplification primer pair comprising an amplification primer of nucleic acid sequence as set forth in SEQ ID NO: 52 and an amplification primer of nucleic acid sequence as set forth in SEQ ID NO: 53.

In certain preferred embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 45, 46, 49, 50, 52 and 54), to detect the presence or absence of exon 51 of the DMD gene the polymerase-based nucleic acid amplification may be configured to amplify a target nucleic acid region using an amplification primer pair comprising an amplification primer of nucleic acid sequence as set forth in SEQ ID NO: 54 and an amplification primer of nucleic acid sequence as set forth in SEQ ID NO: 55.

Hence, in certain particularly preferred embodiments, the polymerase-based nucleic acid amplification may be configured to amplify:
a) to detect the presence or absence of exon 44 of the DMD gene, a target nucleic acid region using an amplification primer pair comprising an amplification primer of nucleic acid sequence as set forth in SEQ ID NO: 11 and an amplification primer of nucleic acid sequence as set forth in SEQ ID NO: 12;
b) to detect the presence or absence of exon 46 of the DMD gene, a target nucleic acid region using an amplification primer pair comprising an amplification primer of nucleic acid sequence as set forth in SEQ ID NO: 13 and an amplification primer of nucleic acid sequence as set forth in SEQ ID NO: 14;
c) to detect the presence or absence of exon 50 of the DMD gene, a target nucleic acid region using an amplification primer pair comprising an amplification primer of nucleic acid sequence as set forth in SEQ ID NO: 15 and an amplification primer of nucleic acid sequence as set forth in SEQ ID NO: 16;
d) to detect the presence or absence of exon 52 of the DMD gene, a target nucleic acid region using an amplification primer pair comprising an amplification primer of nucleic acid sequence as set forth in SEQ ID NO: 17 and an amplification primer of nucleic acid sequence as set forth in SEQ ID NO: 18; and
e) to detect the presence or absence of exon 54 of the DMD gene, a target nucleic acid region using an amplification primer pair comprising an amplification primer of nucleic acid sequence as set forth in SEQ ID NO: 19 and an amplification primer of nucleic acid sequence as set forth in SEQ ID NO: 20;
and/or may be configured to amplify
f) to detect the presence or absence of exon 7 of the DMD gene, a target nucleic acid region using an amplification primer pair comprising an amplification primer of nucleic acid sequence as set forth in SEQ ID NO: 46 and an amplification primer of nucleic acid sequence as set forth in SEQ ID NO: 47;
g) to detect the presence or absence of exon 43 of the DMD gene, a target nucleic acid region using an amplification primer pair comprising an amplification primer of nucleic acid sequence as set forth in SEQ ID NO: 48 and an amplification primer of nucleic acid sequence as set forth in SEQ ID NO: 49;
h) to detect the presence or absence of exon 45 of the DMD gene, a target nucleic acid region using an amplification primer pair comprising an amplification primer of nucleic acid sequence as set forth in SEQ ID NO: 50 and an amplification primer of nucleic acid sequence as set forth in SEQ ID NO: 51;
i) to detect the presence or absence of exon 49 of the DMD gene, a target nucleic acid region using an amplification primer pair comprising an amplification primer of nucleic acid sequence as set forth in SEQ ID NO: 52 and an amplification primer of nucleic acid sequence as set forth in SEQ ID NO: 53; and
j) to detect the presence or absence of exon 51 of the DMD gene, a target nucleic acid region using an amplification primer pair comprising an amplification primer of nucleic acid sequence as set forth in SEQ ID NO: 54 and an amplification primer of nucleic acid sequence as set forth in SEQ ID NO: 55.

The methods as taught herein may be suitably implemented by contacting a sample or at least a portion of nucleic acids isolated from the sample under conditions conducive to polymerase-based nucleic acid amplification with primer pairs configured to amplify the aforementioned DMD exons under said conditions as taught herein. The reference to “conditions conducive to polymerase-based nucleic acid amplification” means that the conditions, such as in particular the composition of the amplification reaction and the physical conditions to which the amplification reaction is subjected (in particular temperature cycling conditions) are sufficient to effect amplification of target nucleic acid regions in the DMD gene by the respective primer pairs as taught herein.

The concentration of the primers in the amplification reaction may be as customary, for example may preferably be about 200 to about 400 nM, preferably about 250 to about 350 nM, such as particularly preferably about 300 nM.

The primer pairs and probes for DMD exons 7, 43, 44, 45, 46, 49, 50, 51, 52 and 54 as taught be certain embodiments of the present invention entail a number of considerable advantages, which make these primers and probes particularly well-suited for multiplex amplifications, specifically for QPCR reactions in which the detection of all these ten exons is multiplexed. For example, the primers display narrowly spaced melting temperatures, which allows the individual amplifications to display optimal efficiency at the same annealing temperature conditions:

Exon 7:
(SEQ ID NO: 46)
Forward primer TGTATGTGTTTTAGGCCAGACC - 63.0° C.,
(SEQ ID NO: 47)
reverse primer TCCAGAAATTTACCAACCTTCA - 62.5° C.,
(SEQ ID NO: 61)
probe 6-FAM- TGG AAT AGT GTG GTT TGC CAG C -BHQ -
68.3° C.;
Exon 43:
(SEQ ID NO: 48)
Forward primer AAAGCTCAGGTCGGATTGAC - 63.6° C.,
(SEQ ID NO: 49)
reverse primer ACCTACCCTTGTCGGTCCTT - 64.0° C.,
(SEQ ID NO: 62)
probe Yakima Yellow- CCA GCT TGA TTT CCA ATG GG -
BHQ - 66.7° C.;
Exon 44:
(SEQ ID NO: 11)
Forward primer TACCTGCAGGCGATTTGAC - 64.3° C.,
(SEQ ID NO: 12)
reverse primer CACCCTTCAGAACCTGATCTTT - 63.6° C.,
(SEQ ID NO: 26)
probe 6-FAM AAATTCCTGAGAATTGGGAACATG - 66.1° C.;
Exon 45:
(SEQ ID NO: 50)
Forward primer GCCTTTTTGGTATCTTACAGGAAC - 63.0°
C.,
(SEQ ID NO: 51)
reverse primer CAGGCTTCCCAATTTTTCC- 63.6° C.,
(SEQ ID NO: 63)
probe ROX- CAG AAC ATT GAA TGC AAC TGG G -BHQ -
66.7° C.;
Exon 46:
(SEQ ID NO: 13)
Forward primer TTTATGGTTGGAGGAAGCAGA - 63.8° C.,
(SEQ ID NO: 14)
reverse primer AATGGGCAGAAAACCAATGA - 64.5° C.,
(SEQ ID NO: 27)
probe Yakima Yellow AACCTGGAAAAGAGCAGCAACT - 65.5°
C.;
Exon 49:
(SEQ ID NO: 52)
Forward primer TTTTCCCCAGGAAACTGAAA - 63.6° C.,
(SEQ ID NO: 53)
reverse primer CCCATTATGAGGTAATGGATATTG - 62.2°
C.,
(SEQ ID NO: 64)
probe Cy5- AAC CGG ATG TGG AAG AGA TTT TG -BHQ -
67.2° C.;
Exon 50:
(SEQ ID NO: 15)
Forward primer CTGAGTGGAAGGCGGTAAAC - 63.9° C.,
(SEQ ID NO: 16)
reverse primer TCTCACCCAGTCATCACTTCA- 63.9° C.,
(SEQ ID NO: 28)
probe ROX ACTTCAAGAGCTGAGGGCAAAG - 66.1° C.;
Exon 51:
(SEQ ID NO: 54)
Forward primer TACCTGCAGGCGATTTGAC - 64.1° C.,
(SEQ ID NO: 55)
reverse primer CACCCTTCAGAACCTGATCTTT - 64.2° C.,
(SEQ ID NO: 65)
probe Atto700- GGG CTT GGA CAG AAC TTA CCG -BHQ -
67.1° C.;
Exon 52:
(SEQ ID NO: 17)
Forward primer AATACACAACGCTGAAGAACCC - 64.3° C.,
(SEQ ID NO: 18)
reverse primer TTGTGTGTCCCATGCTTGTT - 64.6,
(SEQ ID NO: 29)
probe Atto 647N CGCTGCCCAAAATTTGAAAAA - 67.9° C.;
Exon 54:
(SEQ ID NO: 19)
Forward primer TCTATAGCAGTTGGCCAAAGAC - 62.5° C.,
(SEQ ID NO: 20)
reverse primer TCATGGTCCATCCAGTTTCA - 64.5^{O} C.,
(SEQ ID NO: 30)
probe Atto 7000 AATATCAATGCCTCTTGGAGAAGC - 65.6°
C.

Further, the primers and probes display no tendency to form self-dimers or cross-primer dimers.

Also, the primers and probes have been meticulously selected to minimise off-target binding to other sequences in human genome, which could otherwise cause non-specific amplification and/or of sequences other than the respective DMD exons.

In addition, the primers have been selected to produce similarly-sized amplicons, such that the amplification of the different exons can proceed with similar efficiency.

Furthermore, the primers and probes maximally avoid hotspots of mutations or polymorphism in the DMD gene, such that the primers allow for highly universal amplification of the respective DMD exons across most DMD alleles present in human populations.

Numerous different PCR or QPCR protocols are known in the art and can be directly applied or adapted for use with the primers and primer pairs as described herein, and methods as described herein. Generally, in PCR, a target polynucleotide sequence is amplified by reaction with a pair of oligonucleotide primers. The primers hybridise to complementary regions of a target nucleic acid and a DNA polymerase extends the primers to amplify the target sequence, generating an amplification product. The amplification cycle is repeated to increase the concentration of the amplification product.

The reaction can be performed in any thermocycler commonly used for PCR. However, preferred are cyclers with real-time fluorescence measurement capabilities, for example, Smartcycler® (Cepheid, Sunnyvale, Calif.), ABI PRISM 7700® (Applied Biosystems, Foster City, Calif.), Rotor-Gene™ (Corbett Research, Sydney, Australia), Lightcycler® (Roche Diagnostics Corp, Indianapolis, Ind.), iCycler® (Biorad Laboratories, Hercules, Calif.), MX4000® (Stratagene, La Jolla, Calif.), and CFX96 Real-Time PCR system (Biorad).

As used herein, “quantitative PCR” (or “real-time QPCR”) refers to the direct monitoring of the progress of a PCR amplification as it is occurring without the need for repeated sampling of the reaction products. In QPCR, the reaction products may be monitored via a signalling mechanism (e.g., fluorescence) as they are generated and are tracked after the signal rises above a background level but before the reaction reaches a plateau. The number of cycles required to achieve a detectable or “threshold” level of fluorescence (“cycle threshold”, “CT”) varies directly with the concentration of amplifiable targets at the beginning of the PCR process, enabling a measure of signal intensity to provide a measure of the amount of target nucleic acid in a sample in real time.

In preferred embodiments, labelled probes are used to detect the extension product generated by PCR amplification. Any probe format utilising labelled probes as taught herein may be used, e.g., such as SCORPIONS™ probes, sunrise probes, TAQMAN® probes, or molecular beacon probes, as is known in the art or described elsewhere herein. In certain preferred embodiments, the probes and hence detection technology may be TAQMAN® probes.

Methods for setting up a PCR reaction are well known to those skilled in the art. The reaction mixture minimally comprises template nucleic acid (except in the case of a negative control) and oligonucleotide primers and/or probes in combination with suitable buffers, salts, and the like, and an appropriate concentration of a nucleic acid polymerase.

As used herein, “nucleic acid polymerase” refers to an enzyme that catalyses the polymerization of nucleoside triphosphates. Generally, the enzyme will initiate synthesis at the 3′-end of the primer annealed to the target sequence, and will proceed in the 5′-direction along the template until synthesis terminates. An appropriate concentration includes one that catalyses this reaction in the presently described methods. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase. Fusion polymerases with in which a DNA polymerase is fused to a double-stranded DNA binding protein, such as the Sulfolobus sulfactaricus Sso7 protein are also contemplated, e.g., Phusion™ High-Fidelity DNA Polymerase (Thermo Fisher Scientific, Waltham, Mass., USA) or Takyon™ fusion polymerase (Eurogentec, Seraing, Belgium).

In addition to the above components, the reaction mixture of the present methods includes primers, optionally probes, and deoxyribonucleoside triphosphates (dNTPs). Usually the reaction mixture will further comprise four different types of dNTPs corresponding to the four naturally occurring nucleoside bases, i.e., dATP, dTTP, dCTP, and dGTP. In the methods as taught herein, each dNTP will typically be present in an amount ranging from about 10 to 5000 μM, usually from about 20 to 1000 μM, about 100 to 800 μM, or about 300 to 600 μM.

The amplification reaction mixture further includes an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulphate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like.

In preparing the amplification reaction mixture, the various constituent components may be combined in any convenient order. For example, the buffer may be combined with primer, polymerase, and then template nucleic acid, or all of the various constituent components may be combined at the same time to produce the reaction mixture. Alternatively, commercially available premixed reagents can be utilised according to the manufacturer's instructions, or modified to improve reaction conditions (e.g., modification of buffer concentration, cation concentration, or dNTP concentration, as necessary), including, for example, Takyon™ master mix (Eurogentec), TAQMAN® Universal PCR Master Mix (Applied Biosystems), OMNIMIX® or SMARTMIX® (Cepheid), iQ™ Supermix (Bio-Rad Laboratories), Lightcycler® FastStart (Roche Applied Science, Indianapolis, Ind.), or BRILLIANT® QPCR Master Mix (Stratagene, La Jolla, Calif.). Such optimised PCR master mixes are typically sold in concentrated form by vendors of DNA polymerases for PCR use.

Following preparation of the reaction mixture, the reaction mixture is subjected to primer extension reaction conditions (conditions conducive to the amplification), i.e., conditions that permit for polymerase-mediated primer extension by addition of nucleotides to the end of the primer molecule using the template strand as a template. In many embodiments, the primer extension reaction conditions are amplification conditions, which conditions include a plurality of reaction cycles, where each reaction cycle comprises: (1) a denaturation step, (2) an annealing step, and (3) a polymerisation step. The number of reaction cycles will vary depending on the application being performed, but will usually be at least 15, more usually at least 20, and may be as high as 60 or higher, where the number of different cycles will typically range from about 20 to 40, preferably from about 30 to 40, also preferably about 40 such as exactly 40. For methods where more than about 25, usually more than about 30 cycles are performed, it may be convenient or desirable to introduce additional polymerase into the reaction mixture such that conditions suitable for enzymatic primer extension are maintained. The denaturation step comprises heating the reaction mixture to an elevated temperature and maintaining the mixture at the elevated temperature for a period of time sufficient for any double-stranded or hybridised nucleic acid present in the reaction mixture to dissociate. For denaturation, the temperature of the reaction mixture will usually be raised to, and maintained at, a temperature ranging from about 85 to 100° C., usually from about 90 to 98° C., and more usually from about 93 to 96° C., for a period of time ranging from about 3 to 120 sec. Prior to cycling, a longer denaturation step may be included, such as a step of 5 minute denaturation.

Following denaturation, the reaction mixture will be subjected to conditions sufficient for primer annealing to template nucleic acid present in the mixture (if present), and for polymerisation of nucleotides to the primer ends in a manner such that the primer is extended in a 5′ to 3′ direction using the nucleic acid to which it is hybridised as a template, i.e., conditions sufficient for enzymatic production of primer extension product. In this embodiment, the annealing and extension processes occur in the same step. The temperature to which the reaction mixture is lowered to achieve these conditions will usually be chosen to provide optimal efficiency and specificity, and will generally range from about 50 to 75° C., usually from about 55 to 70° C., and more usually from about 60 to 68° C., more particularly around 60° C., e.g., 60° C. or 61° C. Annealing conditions will be maintained for a period of time ranging from about 15 sec to 30 min, usually from about 20 sec to 5 min, or about 30 sec to 2 minutes, or about 1 minute, such as for example 75 seconds.

This step can optionally comprise one of each of an annealing step and an extension step with variation and optimisation of the temperature and length of time for each step. In a two-step annealing and extension, the annealing step is allowed to proceed as above. Following annealing of primer to template nucleic acid, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65 to 75° C., usually from about 67 to 73° C. and maintained for a period of time ranging from about 15 sec to 20 min, usually from about 30 sec to 5 min.

The above cycles of denaturation, annealing, and polymerization may be performed using an automated device, typically known as a thermal cycler, many of which are commercially available (supra).

Further, variations on the exact amounts of the various reagents and on the conditions for the PCR amplification procedure (e.g., buffer conditions, cycling times, etc.) that lead to similar amplification or detection/quantification results are known to those of skill in the art and are considered to be equivalents. In certain embodiments, a hot-start PCR reaction may be performed (e.g., using a hot start Taq DNA polymerase) so as to improve PCR reaction by decreasing background from non-specific amplification and to increase amplification of the desired extension product.

By means of an illustration, in accordance with certain embodiments of the present invention a method may comprise:

(a) obtaining or receiving a sample from a subject;

(b) insofar necessary, processing the sample such as to render it suitable for (compatible with) polymerase chain reaction, or isolating nucleic acids from the sample;

(c) forming a polymerase chain reaction solution comprising (i) the sample or at least a portion of nucleic acids isolated from the sample, (ii) a mixture of nucleoside triphosphate monomers, (iii) a thermostable DNA polymerase such as Taq polymerase in a buffered solution, and (iv) at least amplification primer pairs as taught herein configured to amplify target nucleic acid regions comprising exons 44, 46, 50, 52 and 54 and/or exons 7, 43, 45, 49 and 51, preferably exons 7, 43, 44, 45, 46, 49, 50, 51, 52 and 54 of the DMD gene;

(d) carrying out a polymerase chain reaction on the PCR reaction solution to amplify any of said target nucleic acid regions; and

(e) detecting the amplified target nucleic acid region or regions, whereby the presence or absence of DMD exon 44, 46, 50, 52 or 54 and/or exons 7, 43, 45, 49 and 51, preferably exons 7, 43, 44, 45, 46, 49, 50, 51, 52 and 54 in the sample is detected.

The PCR or QPCR reaction as taught herein may contain various controls. Such controls may include a “no template” negative control, in which primers, buffer, enzyme(s) and other necessary reagents (e.g., magnesium chloride, nucleotides) are cycled in the absence of added test sample. A positive control including a known target nucleic acid (e.g., ribonuclease P protein subunit p30 gene, RPP30) may also be run in parallel. Both positive control and negative control may be included in the amplification reaction. A single reaction may contain either a positive control, a negative control, or a sample template, or a single reaction may contain both a sample template and a positive control. In addition to “no template” controls, negative controls can also include amplification reactions with non-specific target nucleic acid included in the reaction, or can be samples prepared using any or all steps of the sample preparation (from nucleic acid extraction to amplification preparation) without the addition of a test sample.

Positive and negative controls are useful for setting the parameters within which a test sample will be classified as having or not having the exons discussed herein. For example, in a QPCR reaction, the cycle threshold at which an exon is detected in a positive control sample can be used to set the threshold for classifying a sample as “positive” and the cycle threshold at which an exon is detected in a negative control sample can be used to set the threshold for classifying a sample as “negative”. The cycle threshold from a single reaction may be used for each control, or the median or mean of replicate samples may be used. In yet another embodiment, historical control values may be used. The minimum level of detection for each of the negative and the positive controls is typically set at the lower end of the 95% confidence interval of the mean CT across multiple reactions. This value can be adjusted depending on the requirements of the diagnostic assay.

In certain embodiments, in an amplification reaction where the detection of DMD exons 44, 46, 50, 52 and 54 and/or DMD exons 7, 43, 45, 49 and 51, preferably DMD exons 7, 43, 44, 45, 46, 49, 50, 51, 52 and 547, 43, 44, 45, 46, 49, 50, 51, 52 and 54 is multiplexed, the amplification of any one of these exons will also serve as an internal quality control for the amplification reaction as a whole. Where the reaction fails to amplify any of the DMD exons (which may be possible if all exons are deleted), the inclusion of a positive control in the multiplexed reaction can serve to quality control the reaction. If the real-time PCR reader is constrained in the number of channels such that a positive control cannot be included in the multiplexed reaction, it is for example possible to re-run the sample in duplex qPCR reactions, combining each individual DMD exon with a positive control such as the RPP30 reference gene to confirm the DMD exon deletion.

If desired, the identity of the primer extension or amplification product can be confirmed using standard molecular techniques including, for example, a Southern blot assay, a dot blot assay, hybridisation to microarrays, melting curve analyses, sequencing, etc., which will be apparent to a skilled person.

In certain preferred embodiments, the amplification primer pairs as taught herein may be advantageously used in conjunction with oligonucleotide probes configured to hybridise with the target nucleic acid regions. Probes may facilitate detection of the amplified target nucleic acid regions. Accordingly, in certain embodiments, the amplified target nucleic acid regions are detected by oligonucleotide probes configured to hybridise with said target nucleic acid regions.

As used herein, the term “probe” refers to an oligonucleotide, more preferably to a DNA oligonucleotide, which (or part of which) is complementary or sufficiently complementary as defined herein to a sequence comprised in a nucleic acid to be detected by the probe, such that the probe can hybridise (anneal) with said sequence. In particular, probes as intended herewith can hybridise (anneal) with a primer extension product produced by the polymerase-based amplification.

In certain embodiments, probes as taught herein may be defined as configured to hybridise (anneal) within certain recited nucleic acid sequences. In this context, the phrase “hybridise within a nucleic acid” or “hybridise within a nucleic acid sequence” is intended to mean that the probe may anneal to the whole of the recited nucleic acid sequence, or only to a portion of the recited nucleic acid sequence, but does not anneal to sequences adjacent to but outside of the recited nucleic acid sequence.

Probes may be ideally less than or equal to about 50 nucleotides in length, for example less than or equal to about 40, about 30, about 20, or less than about 10 nucleotides in length, e.g., between 10 and 30 or between 15 and 25 nucleotides in length.

A probe as taught herein thus comprises an oligonucleotide sequence which effects the hybridisation (annealing) of the probe with a sequence comprised in a nucleic acid to be detected by the probe. In certain embodiments, a probe does not contain any further oligonucleotide sequence(s). In certain other embodiments, a probe may contain—besides the oligonucleotide sequence which effects the hybridisation of the probe with a sequence comprised in a nucleic acid to be detected by the probe—additional oligonucleotide sequence(s) serving other useful purpose(s). For example but without limitation, such additional oligonucleotide sequence(s) may provide linker sequences allowing to couple a probe with another moiety or moieties, e.g., label(s), or may provide sequences ensuring a certain conformation of a probe, etc.; various options are available to a skilled reader.

By means of example and not limitation, when a probe forms a molecular beacon as known in the art, mutually complementary oligonucleotide extensions are provided at the 5′ and 3′ ends of the probe, one of the oligonucleotide extensions linked to a fluorophore and the other one to a quencher capable of quenching the fluorescent emission of the fluorophore. When the probe is not annealed to the nucleic acid to be detected, the mutually complementary oligonucleotide extensions will form a hairpin structure, whereby the quencher is brought into proximity of the fluorophore and quenches the fluorophore's signal. Conversely, when the probe is annealed to the nucleic acid to be detected, the hairpin structure cannot formed, the quencher is not in proximity of the fluorophore and does not quench the fluorophore's signal, which signal is therefore detectable.

In certain embodiments, the oligonucleotide probes as taught herein may comprise detectable labels. Preferably, such labels may allow for individual detection of each of the amplified target nucleic acid regions.

The term “label” or “labelled” refers to any atom, molecule, moiety or biomolecule that can be used to provide a detectable and preferably quantifiable read-out or property, and that can be attached to or made part of an entity of interest, such as a primer or an oligonucleotide probe. Labels may be suitably detectable by mass spectrometric, spectroscopic, optical, colourimetric, magnetic, photochemical, biochemical, immunochemical or chemical means. A wide variety of labels and conjugation techniques, including direct and indirect labelling, are known and are reported extensively in both the scientific and patent literature. Examples of labels that can be used include radionucleotides, enzymes, substrates, cofactors, inhibitors, fluorescent moieties, intercalators, chemiluminescent moieties, magnetic particles, and the like. For example, labels include without limitation a radioactive isotope (e.g., ³²P, ³³P), ligand, chemiluminescent agent, fluorophore (e.g., fluorescein, tetrachloro-fluorescein, TAMRA, ROX, Cy3, Cy3.5, Cy5, Cy5.5, Texas Red, etc. or the fluorophores used in the present examples), vitamin (e.g., biotin), steroid (e.g., digoxin), enzyme (e.g., HRP, AP, etc.), etc.

Particularly preferably, the detectable labels may comprise distinct fluorophores having distinct excitation and/or emission characteristics, such that each of the amplified target nucleic acid regions can be individually detected by detecting the corresponding fluorophore.

Various probe (or probe plus quencher)-based methodologies for amplification product detection have been developed for QPCR and may be employed in embodiments of the present invention. One example is the TaqMan™ system developed by Applied Biosystems, which relies on the release and detection of a fluorogenic probe during each round of DNA amplification (Holland et al. 1991. Detection of specific polymerase chain reaction product by utilizing the 5′-3′ exonuclease activity of Thermus aquaticus DNA polymerase. PNAS 88: 7276-80). TaqMan™ probes typically contain a 5′-linked fluorophore and a 3′-linked quencher capable of quenching the fluorescent emission of the fluorophore. In another example, systems based on molecular beacons also utilise fluorescence resonance energy transfer (FRET) between a fluorophore and a quencher (see, e.g., Manganelli et al. 2001. Real-time PCR using molecular beacons. Methods Mol Med 54: 295-310; Marras SAE. 2006. Selection of fluorophore and quencher pairs for fluorescent nucleic acid hybridization probes. Methods Mol Biol 335: 3-16; Marras SAE et al. 2006. Real-time assays with molecular beacons and other fluorescent nucleic acid hybridization probes. Clin Chim Acta 363: 48-60 for further discussion of molecular beacons detection). Another system is the Light Upon Extension (LUX™) system commercialised by Invitrogen (Carlsbad, Calif.) and described in detail in Nazarenko et al. 2002 (Nucleic Acids Research 30: e37) and Nazarenko et al. 2002 (Nucleic Acids Research 30: 2089-2095). For additional description of ways to detect and evaluate amplification products in real-time (e.g., using adjacent probes; 5′-nuclease probes such as Taqman™; Light-up probes; Duplex scorpion primers; Amplifluor primers; and further alternative fluorescent hybridisation probe formats) see, e.g., Marras SAE et al. 2006. Real-time assays with molecular beacons and other fluorescent nucleic acid hybridization probes. Clin Chim Acta 363: 48-60, esp. section 6 and references therein.

In certain embodiments, to detect the presence or absence of exon 44 of the DMD gene, the oligonucleotide probe may be configured to hybridise within the nucleic acid sequence TTTAGCATGTTCCCAATTCTCAGGAATTTGTGTC (SEQ ID NO: 21), preferably within positions 2-33, more preferably within positions 3-32, even more preferably within positions 4-31, still more preferably within positions 5-30, and yet more preferably within positions 6-29 of SEQ ID NO: 21.

In certain embodiments, which may optionally and preferably be used in combination with the aforementioned embodiments for the detection of exon 44, to detect the presence or absence of exon 46 of the DMD gene, the oligonucleotide probe may be configured to hybridise within the nucleic acid sequence CTTTTAGTTGCTGCTCTTTTCCAGGTTCAAGT (SEQ ID NO: 22), preferably within positions 2-31, more preferably within positions 3-30, even more preferably within positions 4-29, still more preferably within positions 5-28, and yet more preferably within positions 6-27 of SEQ ID NO: 22.

In certain embodiments, which may optionally and preferably be used in combination with the aforementioned embodiments for the detection of exons 44 and/or 46 (preferably exons 44 and 46), to detect the presence or absence of exon 50 of the DMD gene, the oligonucleotide probe may be configured to hybridise within the nucleic acid sequence GGCTGCTTTGCCCTCAGCTCTTGAAGTAAACG (SEQ ID NO: 23), preferably within positions 2-31, more preferably within positions 3-30, even more preferably within positions 4-29, still more preferably within positions 5-28, and yet more preferably within positions 6-27 of SEQ ID NO: 23.

In certain embodiments, which may optionally and preferably be used in combination with the aforementioned embodiments for the detection of exons 44, 46 and/or 50 (preferably exons 44, 46 and 50), to detect the presence or absence of exon 52 of the DMD gene, the oligonucleotide probe may be configured to hybridise within the nucleic acid sequence TCTTGTTTTTCAAATTTTGGGCAGCGGTAAT (SEQ ID NO: 24), preferably within positions 2-30, more preferably within positions 3-29, even more preferably within positions 4-28, still more preferably within positions 5-27, and yet more preferably within positions 6-26 of SEQ ID NO: 24.

In certain embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene, the oligonucleotide probe may be configured to hybridise within the nucleic acid sequence TGAATGCTTCTCCAAGAGGCATTGATATTCTCTG (SEQ ID NO: 25), preferably within positions 2-33, more preferably within positions 3-32, even more preferably within positions 4-31, still more preferably within positions 5-30, and yet more preferably within positions 6-29 of SEQ ID NO: 25.

In certain embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene, the oligonucleotide probe may be configured to hybridise within the nucleic acid sequence TGACTGCTGGCAAACCACACTATTCCAGTCAA (SEQ ID NO: 56), preferably within positions 2-31, more preferably within positions 3-30, even more preferably within positions 4-29, still more preferably within positions 5-28, and yet more preferably within positions 6-27 of SEQ ID NO: 56.

In certain embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene, the oligonucleotide probe may be configured to hybridise within the nucleic acid sequence TTTTTCCCATTGGAAATCAAGCTGGGAGAG (SEQ ID NO: 57), preferably within positions 2-29, more preferably within positions 3-28, even more preferably within positions 4-27, still more preferably within positions 5-26, and yet more preferably within positions 6-25 of SEQ ID NO: 57.

In certain embodiments, which may optionally and preferably be used in combination with the aforementioned embodiments for the detection of exons 7, 43, 44, 46, 50, 52 and/or 54 (preferably exons 7 and 43, more preferably exons 7, 43, 44, 46, 50, 52 and 54), to detect the presence or absence of exon 45 of the DMD gene, the oligonucleotide probe may be configured to hybridise within the nucleic acid sequence TCTTCCCCAGTTGCATTCAATGTTCTGACAAC (SEQ ID NO: 58), preferably within positions 2-31, more preferably within positions 3-30, even more preferably within positions 4-29, still more preferably within positions 5-28, and yet more preferably within positions 6-27 of SEQ ID NO: 58.

In certain embodiments, which may optionally and preferably be used in combination with the aforementioned embodiments for the detection of exons 7, 43, 44, 45, 46, 50, 52 and/or 54 (preferably exon 7, 43 and 45, more preferably exons 7, 43, 44, 45, 46, 50, 52 and 54), to detect the presence or absence of exon 49 of the DMD gene, the oligonucleotide probe may be configured to hybridise within the nucleic acid sequence TTAGACAAAATCTCTTCCACATCCGGTTGTTTA (SEQ ID NO: 59), preferably within positions 2-32, more preferably within positions 3-31, even more preferably within positions 4-30, still more preferably within positions 5-29, and yet more preferably within positions 6-28 of SEQ ID NO: 59.

In certain embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 45, 46, 49, 50, 52 and 54), to detect the presence or absence of exon 51 of the DMD gene, the oligonucleotide probe may be configured to hybridise within the nucleic acid sequence CCAGTCGGTAAGTTCTGTCCAAGCCCGGTTG (SEQ ID NO: 60), preferably within positions 2-30, more preferably within positions 3-29, even more preferably within positions 4-28, still more preferably within positions 5-27, and yet more preferably within positions 6-26 of SEQ ID NO: 60.

In certain embodiments, to detect the presence or absence of exon 44 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22, 23 or preferably 24 contiguous nucleotides, of the nucleic acid sequence AAATTCCTGAGAATTGGGAACATG (SEQ ID NO: 26) or of a sequence diverging from SEQ ID NO: 26 by addition, deletion or substitution of one or two nucleotides, preferably of one nucleotide, or may comprise, consist essentially of or consist of SEQ ID NO: 26.

In certain embodiments, which may optionally and preferably be used in combination with the aforementioned embodiments for the detection of exon 44, to detect the presence or absence of exon 46 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence AACCTGGAAAAGAGCAGCAACT (SEQ ID NO: 27) or of a sequence diverging from SEQ ID NO: 27 by addition, deletion or substitution of one or two nucleotides, preferably of one nucleotide, or may comprise, consist essentially of or consist of SEQ ID NO: 27.

In certain embodiments, which may optionally and preferably be used in combination with the aforementioned embodiments for the detection of exons 44 and/or 46 (preferably exons 44 and 46), to detect the presence or absence of exon 50 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence ACTTCAAGAGCTGAGGGCAAAG (SEQ ID NO: 28) or of a sequence diverging from SEQ ID NO: 28 by addition, deletion or substitution of one or two nucleotides, preferably of one nucleotide, or may comprise, consist essentially of or consist of SEQ ID NO: 28.

In certain embodiments, which may optionally and preferably be used in combination with the aforementioned embodiments for the detection of exons 44, 46 and/or 50 (preferably exons 44, 46 and 50), to detect the presence or absence of exon 52 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20 or preferably 21 contiguous nucleotides, of the nucleic acid sequence CGCTGCCCAAAATTTGAAAAA (SEQ ID NO: 29) or of a sequence diverging from SEQ ID NO: 29 by addition, deletion or substitution of one or two nucleotides, preferably of one nucleotide, or may comprise, consist essentially of or consist of SEQ ID NO: 29.

In certain embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22, 23 or preferably 24 contiguous nucleotides, of the nucleic acid sequence AATATCAATGCCTCTTGGAGAAGC (SEQ ID NO: 30) or of a sequence diverging from SEQ ID NO: 30 by addition, deletion or substitution of one or two nucleotides, preferably of one nucleotide, or may comprise, consist essentially of or consist of SEQ ID NO: 30.

In certain embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence TGGAATAGTGTGGTTTGCCAGC (SEQ ID NO: 61) or of a sequence diverging from SEQ ID NO: 61 by addition, deletion or substitution of one or two nucleotides, preferably of one nucleotide, or may comprise, consist essentially of or consist of SEQ ID NO: 61.

In certain embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19 or preferably 20 contiguous nucleotides, of the nucleic acid sequence CCAGCTTGATTTCCAATGGG (SEQ ID NO: 62) or of a sequence diverging from SEQ ID NO: 62 by addition, deletion or substitution of one or two nucleotides, preferably of one nucleotide, or may comprise, consist essentially of or consist of SEQ ID NO: 62.

In certain embodiments, which may optionally and preferably be used in combination with the aforementioned embodiments for the detection of exons 7, 43, 44, 46, 50, 52 and/or 54 (preferably exons 7 and 43, more preferably exons 7, 43, 44, 46, 50, 52 and 54), to detect the presence or absence of exon 45 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21 or preferably 22 contiguous nucleotides, of the nucleic acid sequence CAGAACATTGAATGCAACTGGG (SEQ ID NO: 63) or of a sequence diverging from SEQ ID NO: 63 by addition, deletion or substitution of one or two nucleotides, preferably of one nucleotide, or may comprise, consist essentially of or consist of SEQ ID NO: 63.

In certain embodiments, which may optionally and preferably be used in combination with the aforementioned embodiments for the detection of exons 7, 43, 44, 45, 46, 50, 52 and/or 54 (preferably exons 7, 43 and 45, more preferably exons 7, 43, 44, 45, 46, 50, 52 and 54), to detect the presence or absence of exon 49 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20, 21, 22 or preferably 23 contiguous nucleotides, of the nucleic acid sequence AACCGGATGTGGAAGAGATTTTG (SEQ ID NO: 64) or of a sequence diverging from SEQ ID NO: 64 by addition, deletion or substitution of one or two nucleotides, preferably of one nucleotide, or may comprise, consist essentially of or consist of SEQ ID NO: 64.

In certain embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 45, 46, 49, 50, 52 and 54), to detect the presence or absence of exon 51 of the DMD gene the oligonucleotide probe may comprise, consist essentially of or consist of at least 12 contiguous nucleotides, such as for example 12, 13, or 14 contiguous nucleotides, preferably at least 15 contiguous nucleotides, such as 15, 16, or 17 contiguous nucleotides, more preferably at least 18 contiguous nucleotides, such as 18, 19, 20 or preferably 21 contiguous nucleotides, of the nucleic acid sequence GGGCTTGGACAGAACTTACCG (SEQ ID NO: 65) or of a sequence diverging from SEQ ID NO: 65 by addition, deletion or substitution of one or two nucleotides, preferably of one nucleotide, or may comprise, consist essentially of or consist of SEQ ID NO: 65.

In certain preferred embodiments, to detect the presence or absence of exon 44 of the DMD gene the oligonucleotide probe may be of nucleic acid sequence as set forth in SEQ ID NO: 26.

In certain preferred embodiments, which may optionally and preferably be used in combination with the aforementioned embodiments for the detection of exon 44, to detect the presence or absence of exon 46 of the DMD gene the oligonucleotide probe may be of nucleic acid sequence as set forth in SEQ ID NO: 27.

In certain preferred embodiments, which may optionally and preferably be used in combination with the aforementioned embodiments for the detection of exons 44 and/or 46 (preferably exons 44 and 46), to detect the presence or absence of exon 50 of the DMD gene the oligonucleotide probe may be of nucleic acid sequence as set forth in SEQ ID NO: 28.

In certain preferred embodiments, which may optionally and preferably be used in combination with the aforementioned embodiments for the detection of exons 44, 46 and/or 50 (preferably exons 44, 46 and 50), to detect the presence or absence of exon 52 of the DMD gene the oligonucleotide probe may be of nucleic acid sequence as set forth in SEQ ID NO: 29.

In certain preferred embodiments, which may optionally and preferably be used in combination with the aforementioned embodiments for the detection of exons 44, 46, 50 and/or 52 (preferably exons 44, 46, 50 and 52), to detect the presence or absence of exon 54 of the DMD gene the oligonucleotide probe may be of nucleic acid sequence as set forth in SEQ ID NO: 30.

In certain preferred embodiments, which may optionally and preferably be used in combination with the aforementioned embodiments for the detection of exons 44, 46, 50, 52 and/or 54 (preferably exons 44, 46, 50, 52 and 54), to detect the presence or absence of exon 7 of the DMD gene the oligonucleotide probe may be of nucleic acid sequence as set forth in SEQ ID NO: 61.

In certain preferred embodiments, which may optionally and preferably be used in combination with the aforementioned embodiments for the detection of exons 7, 44, 46, 50, 52 and/or 54 (preferably exon 7, more preferably exons 7, 44, 46, 50, 52 and 54), to detect the presence or absence of exon 43 of the DMD gene the oligonucleotide probe may be of nucleic acid sequence as set forth in SEQ ID NO: 62.

In certain preferred embodiments, which may optionally and preferably be used in combination with the aforementioned embodiments for the detection of exons 7, 43, 44, 46, 50, 52 and/or 54 (preferably exons 7 and 43, more preferably exons 7, 43, 44, 46, 50, 52 and 54), to detect the presence or absence of exon 45 of the DMD gene the oligonucleotide probe may be of nucleic acid sequence as set forth in SEQ ID NO: 63.

In certain preferred embodiments, which may optionally and preferably be used in combination with the aforementioned embodiments for the detection of exons 7, 43, 44, 45, 46, 50, 52 and/or 54 (preferably exons 7, 43 and 45, more preferably exons 7, 43, 44, 46, 50, 52 and 54), to detect the presence or absence of exon 49 of the DMD gene the oligonucleotide probe may be of nucleic acid sequence as set forth in SEQ ID NO: 64.

In certain preferred embodiments, which may optionally and preferably be used in combination with the aforementioned embodiments for the detection of exons 7, 43, 44, 45, 46, 49, 50, 52 and/or 54 (preferably exons 7, 43, 45 and 49, more preferably exons 7, 43, 44, 46, 49, 50, 52 and 54), to detect the presence or absence of exon 51 of the DMD gene the oligonucleotide probe may be of nucleic acid sequence as set forth in SEQ ID NO: 65.

Hence, in certain particularly preferred embodiments:
a) to detect the presence or absence of exon 7 of the DMD gene, the oligonucleotide probe is of nucleic acid sequence as set forth in SEQ ID NO: 61;
b) to detect the presence or absence of exon 43 of the DMD gene, the oligonucleotide probe is of nucleic acid sequence as set forth in SEQ ID NO: 62;
c) to detect the presence or absence of exon 44 of the DMD gene, the oligonucleotide probe is of nucleic acid sequence as set forth in SEQ ID NO: 26;
d) to detect the presence or absence of exon 45 of the DMD gene, the oligonucleotide probe is of nucleic acid sequence as set forth in SEQ ID NO: 63;
e) to detect the presence or absence of exon 46 of the DMD gene, the oligonucleotide probe is of nucleic acid sequence as set forth in SEQ ID NO: 27;
f) to detect the presence or absence of exon 49 of the DMD gene, the oligonucleotide probe is of nucleic acid sequence as set forth in SEQ ID NO: 64;
g) to detect the presence or absence of exon 50 of the DMD gene, the oligonucleotide probe is of nucleic acid sequence as set forth in SEQ ID NO: 28;
h) to detect the presence or absence of exon 51 of the DMD gene, the oligonucleotide probe is of nucleic acid sequence as set forth in SEQ ID NO: 65;
i) to detect the presence or absence of exon 52 of the DMD gene, the oligonucleotide probe is of nucleic acid sequence as set forth in SEQ ID NO: 29; and
j) to detect the presence or absence of exon 54 of the DMD gene, the oligonucleotide probe is of nucleic acid sequence as set forth in SEQ ID NO: 30.

The concentration of the probes in the amplification reaction may be as customary, for example may preferably range from about 50 to about 200 nM, such as for example about 80 nM, about 90 nM, about 100 nM, about 110 nM, about 120 nM, about 130 nM, about 140 nM, about 150 nM, about 160 nM, about 170 nM or about 180 nM.

A further aspect provides a set of amplification primer pairs suitable for polymerase-based nucleic acid amplification comprising a set of five amplification primer pairs comprising an amplification primer pair configured to detect the presence or absence of exon 44 of the DMD gene, an amplification primer pair configured to detect the presence or absence of exon 46 of the DMD gene, an amplification primer pair configured to detect the presence or absence of exon 50 of the DMD gene, an amplification primer pair configured to detect the presence or absence of exon 52 of the DMD gene, and an amplification primer pair configured to detect the presence or absence of exon 54 of the DMD gene; and/or

comprising a set of five amplification primer pairs comprising an amplification primer pair configured to detect the presence or absence of exon 7 of the DMD gene, an amplification primer pair configured to detect the presence or absence of exon 43 of the DMD gene, an amplification primer pair configured to detect the presence or absence of exon 45 of the DMD gene, an amplification primer pair configured to detect the presence or absence of exon 49 of the DMD gene, an amplification primer pair configured to detect the presence or absence of exon 51 of the DMD gene,
wherein the respective amplification primer pairs are as defined elsewhere in this specification.

In particular embodiments, the set of amplification primer pairs suitable for polymerase-based nucleic acid amplification comprises an amplification primer pair configured to detect the presence or absence of exon 7 of the DMD gene, an amplification primer pair configured to detect the presence or absence of exon 43 of the DMD gene, an amplification primer pair configured to detect the presence or absence of exon 44 of the DMD gene, an amplification primer pair configured to detect the presence or absence of exon 45 of the DMD gene, an amplification primer pair configured to detect the presence or absence of exon 46 of the DMD gene, an amplification primer pair configured to detect the presence or absence of exon 49 of the DMD gene, an amplification primer pair configured to detect the presence or absence of exon 50 of the DMD gene, an amplification primer pair configured to detect the presence or absence of exon 51 of the DMD gene, an amplification primer pair configured to detect the presence or absence of exon 52 of the DMD gene, and an amplification primer pair configured to detect the presence or absence of exon 54 of the DMD gene, wherein the respective amplification primer pairs are as defined elsewhere in this specification.

The term “set” as in “a set of amplification primer pairs” is synonymous with such terms as “collection”, “group”, “grouping”, “combination”, or “assembly”. The phrase “a set of amplification primer pairs” denotes the set of amplification primer pairs irrespective of whether the primers or primer pairs constituting the set are provided each individually (e.g., each primer or primer pair may be provided within a separate composition), or are provided as several sub-sets together making up the set (e.g., each sub-set of primers or primer pairs may be provided within a separate composition), or are provided as a complete set (e.g., the set of primer pairs may be provided within the same composition).

In certain embodiments, the set of amplification primer pairs may further comprise a set of oligonucleotide probes configured to hybridise with the target nucleic acid regions, preferably wherein the respective oligonucleotide probes are as taught elsewhere in this specification.

Hence, a further aspect provides a set of oligonucleotide probes, wherein the respective oligonucleotide probes are as defined elsewhere in this specification.

The phrase “a set of probes” denotes the set of probes irrespective of whether the probes constituting the set are provided each individually (e.g., each probe may be provided within a separate composition), or are provided as several sub-sets together making up the set (e.g., each sub-set of probes may be provided within a separate composition), or are provided as a complete set (e.g., the set of probes may be provided within the same composition).

The amplification primers or primer pairs comprised in the set may be suitably included in a composition. The same applies for the probes comprised in the set. Hence, a further aspect provides a composition comprising the set of amplification primer pairs as taught above. Another aspect provides a composition comprising the set of probes as taught above. A yet further aspect provides a composition comprising the set of amplification primer pairs and probes as taught above. By means of an example, such compositions may provide the primers and/or probes in a dry (solid, powder) form (e.g., lyophilised or spray dried as known for oligonucleotides in the art) or in a dissolved form (e.g., dissolved in deionised water or in a suitable buffer such as PBS) at a concentration compatible with direct use of the primers or probes or with use as a concentrated stock solution.

The amplification primers or primer pairs comprised in the set may be packaged into kits suitable for DMD gene analysis. Accordingly, another aspect of the invention provides a kit of parts comprising the set of amplification primer pairs and/or the set of oligonucleotide probes as taught herein and optionally further comprising reagents sufficient for formulating a polymerase-based nucleic acid amplification reaction mixture.

For example, such reagents may include one or more or all of thermostable nucleic acid polymerase, preferably thermostable DNA polymerase, such as without limitation Taq polymerase, a mixture of nucleotides, preferably deoxyribonucleotides (dATP, dGTP, dCTP, dTTP), a suitable reaction buffer, source of divalent ions, preferably Mg′ ions, such as magnesium sulphate, and deionised water. The kits may further comprise instructions for using the provided composition in a polymerase-based amplification reaction.

In preferred embodiments, the kit of parts may comprise the set of amplification primer pairs and the set of oligonucleotide probes as taught herein, and optionally further comprise the reagents sufficient for formulating a polymerase-based nucleic acid amplification reaction mixture. The kits may further comprise instructions for using the provided composition in a polymerase-based amplification reaction.

Kits as intended herein may comprise a carrier being compartmentalised to receive in close confinement therein one or more containers, such as tubes or vials. The containers will hold the set of amplification primer pairs and optionally the set of oligonucleotide probes as taught herein. The primers and/or probes may be present in lyophilised form or in an appropriate buffer as necessary. One or more containers may contain one or more enzymes or reagents to be utilised in amplification reactions. These enzymes may be present by themselves or in admixtures, in lyophilized form or in appropriate buffers. The kit may optionally contain any or all additional elements useful to carry out the techniques taught herein, such as buffers, extraction reagents, enzymes, pipettes, plates, nucleic acids, nucleoside triphosphates, filter paper, gel materials, transfer materials, autoradiography supplies, and the like. The various reagent components of the kits may be present in separate containers, or may some or all be pre-combined into a reagent mixture for combination with template nucleic acid.

Instructions for using the provided composition in a polymerase-based amplification reaction may be included in the kits; such as in any one or more of a variety of forms. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, flash memory, etc., on which the information has been recorded. Yet another means that may be present is a website address that may be used via the internet to access the information at a removed site. Any convenient means may be present in the kits.

A further aspect provides a method for diagnosing DMD in a subject, comprising analysing the DMD gene of the subject in accordance with the methods disclosed herein. DMD diagnosis may be concluded when the absence of one or more DMD exons 7, 43, 44, 45, 46, 49, 50, 51, 52 or 54 is detected.

Further provided is a method of treating DMD in a subject in need thereof, such as in particular treating the subject using an exon-skipping therapy of DMD exon 51, 45 or 53, wherein the subject has been identified as having the deletion of one or more DMD exons 44, 46, 50, 52 or 54, using the methods disclosed herein. Further provided is a method of treating DMD in a subject in need thereof, such as in particular treating the subject using an exon-skipping therapy of DMD exon 8, 44, or 50, wherein the subject has been identified as having the deletion of one or more DMD exons 7, 43, 45, 49, or 51, using the methods disclosed herein. Further provided is a method of treating DMD in a subject in need thereof, such as in particular treating the subject using an exon-skipping therapy of DMD exon 8, 44, 50, 51, 45 or 53, wherein the subject has been identified as having the deletion of one or more DMD exons 7, 43, 44, 45, 46, 49, 50, 51, 52 or 54, using the methods disclosed herein.

A related aspect provides a DMD exon-skipping therapeutic agent, particularly an agent capable of inducing skipping of DMD exon 51, 45 or 53, wherein the subject has been identified as having the deletion of one or more DMD exons 44, 46, 50, 52 or 54, using the methods disclosed herein. A related aspect provides a DMD exon-skipping therapeutic agent, particularly an agent capable of inducing skipping of DMD exon 8, 44, or 50, wherein the subject has been identified as having the deletion of one or more DMD exons 7, 43, 45, 49, or 51, using the methods disclosed herein. A related aspect provides a DMD exon-skipping therapeutic agent, particularly an agent capable of inducing skipping of DMD exon 8, 44, 50, 51, 45 or 53, wherein the subject has been identified as having the deletion of one or more DMD exons 7, 43, 44, 45, 46, 49, 50, 51, 52 or 54, using the methods disclosed herein.

The present application also provides aspects and embodiments as set forth in the following Statements:

Statement 1. A method for analysing the Duchenne Muscular Dystrophy (DMD) gene in a sample containing genetic material of a subject, wherein the method detects the presence or absence of at least exons 44, 46, 50, 52 and 54, and/or at least exons 7, 43, 45, 49 and 51, of the DMD gene in the genetic material of the subject, wherein the detection of the presence or absence of the exons comprises multiplex polymerase-based nucleic acid amplification.

Statement 2. The method according to Statement 1, wherein the sample is blood.

Statement 3. A method for analysing the Duchenne Muscular Dystrophy (DMD) gene in a sample containing genetic material of a subject, wherein the method detects the presence or absence of at least exons 44, 46, 50, 52 and 54, and/or at least exons 7, 43, 45, 49 and 51, of the DMD gene in the genetic material of the subject, and wherein the sample is blood.

Statement 4. The method according to any one of Statements 1 or 2, wherein the multiplex polymerase-based nucleic acid amplification is multiplex polymerase chain reaction (PCR).

Statement 5. The method according to any one of Statements 1, 2 or 4, wherein the multiplex polymerase-based nucleic acid amplification is multiplex real-time quantitative amplification, preferably multiplex real-time quantitative PCR (qPCR).

Statement 6. The method according to any one of Statements 1, 2, 4 or 5, wherein the detection of the presence or absence of at least exons 44, 46, 50, 52 and 54, and/or at least exons 7, 43, 45, 49 and 51, of the DMD gene in the genetic material of the subject is multiplexed in a single polymerase-based nucleic acid amplification reaction.

Statement 7. The method according to any one of Statements 1, 2 or 4 to 6, wherein the polymerase-based nucleic acid amplification is configured to amplify:
a) to detect the presence or absence of exon 44 of the DMD gene, a target nucleic acid region using an amplification primer pair comprising an amplification primer configured to hybridise within the nucleic acid sequence GATCTGTCAAATCGCCTGCAGGTAAAAGC (SEQ ID NO: 1) and an amplification primer configured to hybridise within the nucleic acid sequence TTCTTAAAGATCAGGTTCTGAAGGGTGATGGA (SEQ ID NO: 2);
b) to detect the presence or absence of exon 46 of the DMD gene, a target nucleic acid region using an amplification primer pair comprising an amplification primer configured to hybridise within the nucleic acid sequence TGTTATCTGCTTCCTCCAACCATAAAACAAA (SEQ ID NO: 3) and an amplification primer configured to hybridise within the nucleic acid sequence TTCAATCATTGGTTTTCTGCCCATTAGGTT (SEQ ID NO: 4);
c) to detect the presence or absence of exon 50 of the DMD gene, a target nucleic acid region using an amplification primer pair comprising an amplification primer configured to hybridise within the nucleic acid sequence AAACGGTTTACCGCCTTCCACTCAGAGCTC (SEQ ID NO: 5) and an amplification primer configured to hybridise within the nucleic acid sequence AACTATGAAGTGATGACTGGGTGAGAGAGAA (SEQ ID NO: 6);
d) to detect the presence or absence of exon 52 of the DMD gene, a target nucleic acid region using an amplification primer pair comprising an amplification primer configured to hybridise within the nucleic acid sequence TATCAGGGTTCTTCAGCGTTGTGTATTCCTTT (SEQ ID NO: 7) and an amplification primer configured to hybridise within the nucleic acid sequence TTTTTAACAAGCATGGGACACACAAAGCAA (SEQ ID NO: 8); and
e) to detect the presence or absence of exon 54 of the DMD gene, a target nucleic acid region using an amplification primer pair comprising an amplification primer configured to hybridise within the nucleic acid sequence CGGAGGTCTTTGGCCAACTGCTATAGATTTTT (SEQ ID NO: 9) and an amplification primer configured to hybridise within the nucleic acid sequence GGTGGTGAAACTGGATGGACCATGAGGATT (SEQ ID NO: 10); and/or
f) to detect the presence or absence of exon 7 of the DMD gene, a target nucleic acid region using an amplification primer pair comprising an amplification primer configured to hybridise within the nucleic acid sequence AAATAGGTCTGGCCTAAAACACATACACATAC (SEQ ID NO: 36) and an amplification primer configured to hybridise within the nucleic acid sequence GATCCTGAAGGTTGGTAAATTTCTGGACTACC (SEQ ID NO: 37);
g) to detect the presence or absence of exon 43 of the DMD gene, a target nucleic acid region using an amplification primer pair comprising an amplification primer configured to hybridise within the nucleic acid sequence ATAATGTCAATCCGACCTGAGCTTTGTTGT (SEQ ID NO: 38) and an amplification primer configured to hybridise within the nucleic acid sequence TGTACAAGGACCGACAAGGGTAGGTAACAC (SEQ ID NO: 39);
h) to detect the presence or absence of exon 45 of the DMD gene, a target nucleic acid region using an amplification primer pair comprising an amplification primer configured to hybridise within the nucleic acid sequence CTGGAGTTCCTGTAAGATACCAAAAAGGCAAAAC (SEQ ID NO: 40) and an amplification primer configured to hybridise within the nucleic acid sequence CTACAGGAAAAATTGGGAAGCCTGAATCT (SEQ ID NO: 41);
i) to detect the presence or absence of exon 49 of the DMD gene, a target nucleic acid region using an amplification primer pair comprising an amplification primer configured to hybridise within the nucleic acid sequence TGCTATTTCAGTTTCCTGGGGAAAAGAACC (SEQ ID NO: 42) and an amplification primer configured to hybridise within the nucleic acid sequence TCTAGCAATATCCATTACCTCATAATGGGTTATG (SEQ ID NO: 43); and
j) to detect the presence or absence of exon 51 of the DMD gene, a target nucleic acid region using an amplification primer pair comprising an amplification primer configured to hybridise within the nucleic acid sequence CCACAGGTTGTGTCACCAGAGTAACAGTCTGAGT (SEQ ID NO: 44) and an amplification primer configured to hybridise within the nucleic acid sequence TTATAAAATCACAGAGGGTGATGGTGGGTGA (SEQ ID NO: 45).

Statement 8. The method according to any one of Statements 1, 2 or 4 to 7, wherein the polymerase-based nucleic acid amplification is configured to amplify:
a) to detect the presence or absence of exon 44 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence TACCTGCAGGCGATTTGAC (SEQ ID NO: 11) or of a sequence diverging from SEQ ID NO: 11 by addition, deletion or substitution of one or two nucleotides, or comprising, consisting essentially of or consisting of SEQ ID NO: 11; and
   an amplification primer comprising at least 12 contiguous nucleotides, preferably at least 15 contiguous nucleotides, more preferably at least 18 contiguous nucleotides of the nucleic acid sequence CACCCTTCAGAACCTGATCTTT (SEQ ID NO: 12) or of a sequence diverging from SEQ ID NO: 12 by addition, deletion or substitution of one or two nucleotides, or comprising, consisting essentially of or consisting of SEQ ID NO: 12;
b) to detect the presence or absence of exon 46 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence TTTATGGTTGGAGGAAGCAGA (SEQ ID NO: 13) or of a sequence diverging from SEQ ID NO: 13 by addition, deletion or substitution of one or two nucleotides, or comprising, consisting essentially of or consisting of SEQ ID NO: 13; and
   an amplification primer comprising at least 12 contiguous nucleotides, preferably at least 15 contiguous nucleotides, more preferably at least 18 contiguous nucleotides of the nucleic acid sequence AATGGGCAGAAAACCAATGA (SEQ ID NO: 14) or of a sequence diverging from SEQ ID NO: 14 by addition, deletion or substitution of one or two nucleotides, or comprising, consisting essentially of or consisting of SEQ ID NO: 14;
c) to detect the presence or absence of exon 50 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence CTGAGTGGAAGGCGGTAAAC (SEQ ID NO: 15) or of a sequence diverging from SEQ ID NO: 15 by addition, deletion or substitution of one or two nucleotides, or comprising, consisting essentially of or consisting of SEQ ID NO: 15; and
   an amplification primer comprising at least 12 contiguous nucleotides, preferably at least 15 contiguous nucleotides, more preferably at least 18 contiguous nucleotides of the nucleic acid sequence TCTCACCCAGTCATCACTTCA (SEQ ID NO: 16) or of a sequence diverging from SEQ ID NO: 16 by addition, deletion or substitution of one or two nucleotides, or comprising, consisting essentially of or consisting of SEQ ID NO: 16;
d) to detect the presence or absence of exon 52 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence AATACACAACGCTGAAGAACCC (SEQ ID NO: 17) or of a sequence diverging from SEQ ID NO: 17 by addition, deletion or substitution of one or two nucleotides, or comprising, consisting essentially of or consisting of SEQ ID NO: 17; and
   an amplification primer comprising at least 12 contiguous nucleotides, preferably at least 15 contiguous nucleotides, more preferably at least 18 contiguous nucleotides of the nucleic acid sequence TTGTGTGTCCCATGCTTGTT (SEQ ID NO: 18) or of a sequence diverging from SEQ ID NO: 18 by addition, deletion or substitution of one or two nucleotides, or comprising, consisting essentially of or consisting of SEQ ID NO: 18; and
e) to detect the presence or absence of exon 54 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence TCTATAGCAGTTGGCCAAAGAC (SEQ ID NO: 19) or of a sequence diverging from SEQ ID NO: 19 by addition, deletion or substitution of one or two nucleotides, or comprising, consisting essentially of or consisting of SEQ ID NO: 19; and
   an amplification primer comprising at least 12 contiguous nucleotides, preferably at least 15 contiguous nucleotides, more preferably at least 18 contiguous nucleotides of the nucleic acid sequence TCATGGTCCATCCAGTTTCA (SEQ ID NO: 20) or of a sequence diverging from SEQ ID NO: 20 by addition, deletion or substitution of one or two nucleotides, or comprising, consisting essentially of or consisting of SEQ ID NO: 20; and/or
f) to detect the presence or absence of exon 7 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence TGTATGTGTTTTAGGCCAGACC (SEQ ID NO: 46) or of a sequence diverging from SEQ ID NO: 46 by addition, deletion or substitution of one or two nucleotides, or comprising, consisting essentially of or consisting of SEQ ID NO: 46; and
   an amplification primer comprising at least 12 contiguous nucleotides, preferably at least 15 contiguous nucleotides, more preferably at least 18 contiguous nucleotides of the nucleic acid sequence TCCAGAAATTTACCAACCTTCA (SEQ ID NO: 47) or of a sequence diverging from SEQ ID NO: 47 by addition, deletion or substitution of one or two nucleotides, or comprising, consisting essentially of or consisting of SEQ ID NO: 47;
g) to detect the presence or absence of exon 43 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence AAAGCTCAGGTCGGATTGAC (SEQ ID NO: 48) or of a sequence diverging from SEQ ID NO: 48 by addition, deletion or substitution of one or two nucleotides, or comprising, consisting essentially of or consisting of SEQ ID NO: 48; and
   an amplification primer comprising at least 12 contiguous nucleotides, preferably at least 15 contiguous nucleotides, more preferably at least 18 contiguous nucleotides of the nucleic acid sequence ACCTACCCTTGTCGGTCCTT (SEQ ID NO: 49) or of a sequence diverging from SEQ ID NO: 49 by addition, deletion or substitution of one or two nucleotides, or comprising, consisting essentially of or consisting of SEQ ID NO: 49;
h) to detect the presence or absence of exon 45 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence GCCTTTTTGGTATCTTACAGGAAC (SEQ ID NO: 50) or of a sequence diverging from SEQ ID NO: 50 by addition, deletion or substitution of one or two nucleotides, or comprising, consisting essentially of or consisting of SEQ ID NO: 50; and
   an amplification primer comprising at least 12 contiguous nucleotides, preferably at least 15 contiguous nucleotides, more preferably at least 18 contiguous nucleotides of the nucleic acid sequence CAGGCTTCCCAATTTTTCC (SEQ ID NO: 51) or of a sequence diverging from SEQ ID NO: 51 by addition, deletion or substitution of one or two nucleotides, or comprising, consisting essentially of or consisting of SEQ ID NO: 51;
i) to detect the presence or absence of exon 49 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence TTTTCCCCAGGAAACTGAAA (SEQ ID NO: 52) or of a sequence diverging from SEQ ID NO: 52 by addition, deletion or substitution of one or two nucleotides, or comprising, consisting essentially of or consisting of SEQ ID NO: 52; and
   an amplification primer comprising at least 12 contiguous nucleotides, preferably at least 15 contiguous nucleotides, more preferably at least 18 contiguous nucleotides of the nucleic acid sequence CCCATTATGAGGTAATGGATATTG (SEQ ID NO: 53) or of a sequence diverging from SEQ ID NO: 53 by addition, deletion or substitution of one or two nucleotides, or comprising, consisting essentially of or consisting of SEQ ID NO: 53; and
j) to detect the presence or absence of exon 51 of the DMD gene, a target nucleic acid region using an amplification primer pair comprising:
   an amplification primer comprising at least 12 contiguous nucleotides, preferably at least 15 contiguous nucleotides, more preferably at least 18 contiguous nucleotides of the nucleic acid sequence GACTGTTACTCTGGTGACACAACC (SEQ ID NO: 54) or of a sequence diverging from SEQ ID NO: 54 by addition, deletion or substitution of one or two nucleotides, or comprising, consisting essentially of or consisting of SEQ ID NO: 54; and
   an amplification primer comprising at least 12 contiguous nucleotides, preferably at least 15 contiguous nucleotides, more preferably at least 18 contiguous nucleotides of the nucleic acid sequence CACCATCACCCTCTGTGATTT (SEQ ID NO: 55) or of a sequence diverging from SEQ ID NO: 55 by addition, deletion or substitution of one or two nucleotides, or comprising, consisting essentially of or consisting of SEQ ID NO: 55.

Statement 9. The method according to any one of Statements 1, 2 or 4 to 8, wherein the polymerase-based nucleic acid amplification is configured to amplify:
a) to detect the presence or absence of exon 44 of the DMD gene, a target nucleic acid region using an amplification primer pair comprising an amplification primer of nucleic acid sequence as set forth in SEQ ID NO: 11 and an amplification primer of nucleic acid sequence as set forth in SEQ ID NO: 12;
b) to detect the presence or absence of exon 46 of the DMD gene, a target nucleic acid region using an amplification primer pair comprising an amplification primer of nucleic acid sequence as set forth in SEQ ID NO: 13 and an amplification primer of nucleic acid sequence as set forth in SEQ ID NO: 14;
c) to detect the presence or absence of exon 50 of the DMD gene, a target nucleic acid region using an amplification primer pair comprising an amplification primer of nucleic acid sequence as set forth in SEQ ID NO: 15 and an amplification primer of nucleic acid sequence as set forth in SEQ ID NO: 16;
d) to detect the presence or absence of exon 52 of the DMD gene, a target nucleic acid region using an amplification primer pair comprising an amplification primer of nucleic acid sequence as set forth in SEQ ID NO: 17 and an amplification primer of nucleic acid sequence as set forth in SEQ ID NO: 18; and
e) to detect the presence or absence of exon 54 of the DMD gene, a target nucleic acid region using an amplification primer pair comprising an amplification primer of nucleic acid sequence as set forth in SEQ ID NO: 19 and an amplification primer of nucleic acid sequence as set forth in SEQ ID NO: 20; and/or
f) to detect the presence or absence of exon 7 of the DMD gene, a target nucleic acid region using an amplification primer pair comprising an amplification primer of nucleic acid sequence as set forth in SEQ ID NO: 46 and an amplification primer of nucleic acid sequence as set forth in SEQ ID NO: 47;
g) to detect the presence or absence of exon 43 of the DMD gene, a target nucleic acid region using an amplification primer pair comprising an amplification primer of nucleic acid sequence as set forth in SEQ ID NO: 48 and an amplification primer of nucleic acid sequence as set forth in SEQ ID NO: 49;
h) to detect the presence or absence of exon 45 of the DMD gene, a target nucleic acid region using an amplification primer pair comprising an amplification primer of nucleic acid sequence as set forth in SEQ ID NO: 50 and an amplification primer of nucleic acid sequence as set forth in SEQ ID NO: 51;
i) to detect the presence or absence of exon 49 of the DMD gene, a target nucleic acid region using an amplification primer pair comprising an amplification primer of nucleic acid sequence as set forth in SEQ ID NO: 52 and an amplification primer of nucleic acid sequence as set forth in SEQ ID NO: 53; and
j) to detect the presence or absence of exon 51 of the DMD gene, a target nucleic acid region using an amplification primer pair comprising an amplification primer of nucleic acid sequence as set forth in SEQ ID NO: 54 and an amplification primer of nucleic acid sequence as set forth in SEQ ID NO: 55.

Statement 10. The method according to any one of Statements 7 to 9, wherein the amplified target nucleic acid regions are detected by oligonucleotide probes configured to hybridise with said target nucleic acid regions, preferably wherein the oligonucleotide probes comprise detectable labels allowing for individual detection of each of the amplified target nucleic acid regions, more preferably wherein the detectable labels comprise distinct fluorophores having distinct excitation and/or emission characteristics, such that each of the amplified target nucleic acid regions can be individually detected by detecting the corresponding fluorophore.

Statement 11. The method according to Statement 10, wherein:
a) to detect the presence or absence of exon 44 of the DMD gene, the oligonucleotide probe is configured to hybridise within the nucleic acid sequence

(SEQ ID NO: 21)
TTTAGCATGTTCCCAATTCTCAGGAATTTGTGTC;

b) to detect the presence or absence of exon 46 of the DMD gene, the oligonucleotide probe is configured to hybridise within the nucleic acid sequence

(SEQ ID NO: 22)
CTTTTAGTTGCTGCTCTTTTCCAGGTTCAAGT;

c) to detect the presence or absence of exon 50 of the DMD gene, the oligonucleotide probe is configured to hybridise within the nucleic acid sequence

(SEQ ID NO: 23)
GGCTGCTTTGCCCTCAGCTCTTGAAGTAAACG;

d) to detect the presence or absence of exon 52 of the DMD gene, the oligonucleotide probe is configured to hybridise within the nucleic acid sequence

(SEQ ID NO: 24)
TCTTGTTTTTCAAATTTTGGGCAGCGGTAAT;

e) to detect the presence or absence of exon 54 of the DMD gene, the oligonucleotide probe is configured to hybridise within the nucleic acid sequence TGAATGCTTCTCCAAGAGGCATTGATATTCTCTG (SEQ ID NO: 25); and/or
f) to detect the presence or absence of exon 7 of the DMD gene, a target nucleic acid region using an amplification primer pair comprising an amplification primer of nucleic acid sequence as set forth in SEQ ID NO: 46 and an amplification primer of nucleic acid sequence as set forth in SEQ ID NO: 47;
g) to detect the presence or absence of exon 43 of the DMD gene, a target nucleic acid region using an amplification primer pair comprising an amplification primer of nucleic acid sequence as set forth in SEQ ID NO: 48 and an amplification primer of nucleic acid sequence as set forth in SEQ ID NO: 49;
h) to detect the presence or absence of exon 45 of the DMD gene, a target nucleic acid region using an amplification primer pair comprising an amplification primer of nucleic acid sequence as set forth in SEQ ID NO: 50 and an amplification primer of nucleic acid sequence as set forth in SEQ ID NO: 51;
i) to detect the presence or absence of exon 49 of the DMD gene, a target nucleic acid region using an amplification primer pair comprising an amplification primer of nucleic acid sequence as set forth in SEQ ID NO: 52 and an amplification primer of nucleic acid sequence as set forth in SEQ ID NO: 53; and
j) to detect the presence or absence of exon 51 of the DMD gene, a target nucleic acid region using an amplification primer pair comprising an amplification primer of nucleic acid sequence as set forth in SEQ ID NO: 54 and an amplification primer of nucleic acid sequence as set forth in SEQ ID NO: 55.

Statement 12. The method according to Statement 10 or 11, wherein:
a) to detect the presence or absence of exon 44 of the DMD gene, the oligonucleotide probe comprises at least 12 contiguous nucleotides, preferably at least 15 contiguous nucleotides, more preferably at least 18 contiguous nucleotides of the nucleic acid sequence AAATTCCTGAGAATTGGGAACATG (SEQ ID NO: 26) or of a sequence diverging from SEQ ID NO: 26 by addition, deletion or substitution of one or two nucleotides, or comprises, consists essentially of or consists of SEQ ID NO: 26, preferably the oligonucleotide probe is of nucleic acid sequence as set forth in SEQ ID NO: 26;
b) to detect the presence or absence of exon 46 of the DMD gene, the oligonucleotide probe comprises at least 12 contiguous nucleotides, preferably at least 15 contiguous nucleotides, more preferably at least 18 contiguous nucleotides of the nucleic acid sequence AACCTGGAAAAGAGCAGCAACT (SEQ ID NO: 27) or of a sequence diverging from SEQ ID NO: 27 by addition, deletion or substitution of one or two nucleotides, or comprises, consists essentially of or consists of SEQ ID NO: 27, preferably the oligonucleotide probe is of nucleic acid sequence as set forth in SEQ ID NO: 27;
c) to detect the presence or absence of exon 50 of the DMD gene, the oligonucleotide probe comprises at least 12 contiguous nucleotides, preferably at least 15 contiguous nucleotides, more preferably at least 18 contiguous nucleotides of the nucleic acid sequence ACTTCAAGAGCTGAGGGCAAAG (SEQ ID NO: 28) or of a sequence diverging from SEQ ID NO: 28 by addition, deletion or substitution of one or two nucleotides, or comprises, consists essentially of or consists of SEQ ID NO: 28, preferably the oligonucleotide probe is of nucleic acid sequence as set forth in SEQ ID NO: 28;
d) to detect the presence or absence of exon 52 of the DMD gene, the oligonucleotide probe comprises at least 12 contiguous nucleotides, preferably at least 15 contiguous nucleotides, more preferably at least 18 contiguous nucleotides of the nucleic acid sequence CGCTGCCCAAAATTTGAAAAA (SEQ ID NO: 29) or of a sequence diverging from SEQ ID NO: 29 by addition, deletion or substitution of one or two nucleotides, or comprises, consists essentially of or consists of SEQ ID NO: 29, preferably the oligonucleotide probe is of nucleic acid sequence as set forth in SEQ ID NO: 29; and
e) to detect the presence or absence of exon 54 of the DMD gene, the oligonucleotide probe comprises at least 12 contiguous nucleotides, preferably at least 15 contiguous nucleotides, more preferably at least 18 contiguous nucleotides of the nucleic acid sequence AATATCAATGCCTCTTGGAGAAGC (SEQ ID NO: 30) or of a sequence diverging from SEQ ID NO: 30 by addition, deletion or substitution of one or two nucleotides, or comprises, consists essentially of or consists of SEQ ID NO: 30, preferably the oligonucleotide probe is of nucleic acid sequence as set forth in SEQ ID NO: 30; and/or
f) to detect the presence or absence of exon 7 of the DMD gene, the oligonucleotide probe comprises at least 12 contiguous nucleotides, preferably at least 15 contiguous nucleotides, more preferably at least 18 contiguous nucleotides of the nucleic acid sequence TGGAATAGTGTGGTTTGCCAGC (SEQ ID NO: 61) or of a sequence diverging from SEQ ID NO: 61 by addition, deletion or substitution of one or two nucleotides, or comprises, consists essentially of or consists of SEQ ID NO: 61, preferably the oligonucleotide probe is of nucleic acid sequence as set forth in SEQ ID NO: 61;
g) to detect the presence or absence of exon 43 of the DMD gene, the oligonucleotide probe comprises at least 12 contiguous nucleotides, preferably at least 15 contiguous nucleotides, more preferably at least 18 contiguous nucleotides of the nucleic acid sequence CCAGCTTGATTTCCAATGGG (SEQ ID NO: 62) or of a sequence diverging from SEQ ID NO: 62 by addition, deletion or substitution of one or two nucleotides, or comprises, consists essentially of or consists of SEQ ID NO: 62, preferably the oligonucleotide probe is of nucleic acid sequence as set forth in SEQ ID NO: 62;
h) to detect the presence or absence of exon 45 of the DMD gene, the oligonucleotide probe comprises at least 12 contiguous nucleotides, preferably at least 15 contiguous nucleotides, more preferably at least 18 contiguous nucleotides of the nucleic acid sequence CAGAACATTGAATGC AACTGGG (SEQ ID NO: 63) or of a sequence diverging from SEQ ID NO: 63 by addition, deletion or substitution of one or two nucleotides, or comprises, consists essentially of or consists of SEQ ID NO: 63, preferably the oligonucleotide probe is of nucleic acid sequence as set forth in SEQ ID NO: 63;
i) to detect the presence or absence of exon 49 of the DMD gene, the oligonucleotide probe comprises at least 12 contiguous nucleotides, preferably at least 15 contiguous nucleotides, more preferably at least 18 contiguous nucleotides of the nucleic acid sequence AACCGGATGTGGAAGAGATTTTG (SEQ ID NO: 64) or of a sequence diverging from SEQ ID NO: 64 by addition, deletion or substitution of one or two nucleotides, or comprises, consists essentially of or consists of SEQ ID NO: 64, preferably the oligonucleotide probe is of nucleic acid sequence as set forth in SEQ ID NO: 64; and
j) to detect the presence or absence of exon 51 of the DMD gene, the oligonucleotide probe comprises at least 12 contiguous nucleotides, preferably at least 15 contiguous nucleotides, more preferably at least 18 contiguous nucleotides of the nucleic acid sequence GGGCTTGGACAGAACTTACCG (SEQ ID NO: 65) or of a sequence diverging from SEQ ID NO: 65 by addition, deletion or substitution of one or two nucleotides, or comprises, consists essentially of or consists of SEQ ID NO: 65, preferably the oligonucleotide probe is of nucleic acid sequence as set forth in SEQ ID NO: 65.

Statement 13. The method according to any one of Statements 1 to 12, wherein the method detects the presence or absence of at least exons that are selected such that a deletion in the DMD gene spanning one or more of the exons shifts the reading frame of the DMD gene downstream of the deletion and the reading frame can be restored by exon-skipping therapy.

Statement 14. The method according to any one of Statements 1 to 13, wherein the subject is a mammal, preferably a human, more preferably a neonate.

Statement 15. The method according to any one of Statements 1 to 14, wherein the sample is whole blood or any fraction of blood containing DNA, or dried blood, more preferably a dried blood spot.

Statement 16. A set of amplification primer pairs suitable for polymerase-based nucleic acid amplification, comprising a set of five amplification primer pairs comprising an amplification primer pair configured to detect the presence or absence of exon 44 of the DMD gene, an amplification primer pair configured to detect the presence or absence of exon 46 of the DMD gene, an amplification primer pair configured to detect the presence or absence of exon 50 of the DMD gene, an amplification primer pair configured to detect the presence or absence of exon 52 of the DMD gene, and an amplification primer pair configured to detect the presence or absence of exon 54 of the DMD gene, and/or a set of five amplification primer pairs comprising an amplification primer pair configured to detect the presence or absence of exon 7 of the DMD gene, an amplification primer pair configured to detect the presence or absence of exon 43 of the DMD gene, an amplification primer pair configured to detect the presence or absence of exon 45 of the DMD gene, an amplification primer pair configured to detect the presence or absence of exon 49 of the DMD gene, and an amplification primer pair configured to detect the presence or absence of exon 51 of the DMD gene, wherein the respective amplification primer pairs are as defined in any one of Statements 7 to 9.

Statement 17. The set of amplification primer pairs according to Statement 16, further comprising a set of oligonucleotide probes configured to hybridise with the target nucleic acid regions, preferably wherein the respective oligonucleotide probes are as defined in any one of Statements 10 to 12.

Statement 18. A set of oligonucleotide probes, wherein the respective oligonucleotide probes are as defined in any one of Statements 10 to 12.

Statement 19. A composition comprising the set of amplification primer pairs and/or the set of oligonucleotide probes according to any one of Statements 16 to 18.

Statement 20. A kit of parts comprising the set of amplification primer pairs and/or the set of oligonucleotide probes according to any one of Statements 16 to 18, and optionally further comprising reagents sufficient for formulating a polymerase-based nucleic acid amplification reaction mixture.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments ofthe invention are further supported by the following non-limiting examples.

### EXAMPLES

### Example 1—DMD Genotyping Assay

DNA from human Duchenne Muscular Dystrophy (DMD) and control samples were collected either from dried blood spots (DBS) or from EDTA whole blood. In total, 120 samples were collected and were classified into three different subgroups.

The “deleted group” consisted of 51 male patients with a clinical DMD phenotype. Amongst them, 34 had a confirmed deletion of one or more DMD exons selected from exons 44, 46, 50, 52 or 54. The other 17 male patients carried a deletion of another DMD exon.

The “carrier group” included 50 females with one deleted DMD allele, of whom 32 carried a deletion that overlapped at least one of exons 44, 46, 50, 52 or 54, while 18 had a deletion of any other DMD exon.

The “control group” consisted of 19 individuals with a normal DMD gene sequence.

The DMD genotyping assay was designed and validated to detect hemi-(homo)-zygotic deletions of DMD exons 44, 46, 50, 52 and 54, relying on a multiplex quantitative polymerase chain reaction (qPCR) assay.

DNA was extracted from one 3.1-mm dried blood spot according to the protocol described by Saavedra-Matiz et al. (Clin Chem. 2013, vol. 59(7), 1045-1051). Isolated DNA was not quantitated, and 1 μL of freshly extracted DNA was mixed with 5×Takyon master mix (Eurogentec, Liege, Belgium), primers and probes in a total volume of 25 μL.

The sequences and concentrations of primers and probes (in the present examples TAQMAN® probes and detection strategy were used) are shown in Table 1. Assays were run on a CFX96 Real-Time PCR System (Bio-Rad Laboratories, Hercules, Calif., USA) under the following conditions: 95° C. for 5 min, followed by 40 cycles of 95° C. for 30 sec and 61° C. for 75 sec.

TABLE 1
qPCR primers and probes used to amplify and identify human DMD exons 44, 46, 50,
52 and 54.

| | | |
|---|---|---|
| DMD target | | Concentration |
| exon | Sequence | (nmol/L) |

DMD exon 44

| | | |
|---|---|---|
| Forward primer | 5′-TACCTGCAGGCGATTTGAC-3′ | 300 |
| | SEQ ID NO: 11 | |
| Reverse primer | 5′-CACCCTTCAGAACCTGATCTTT-3′ | 300 |
| | SEQ ID NO : 12 | |
| Probe | 5′-AAATTCCTGAGAATTGGGAACATG-3′ | 130 |
| | SEQ ID NO: 26 | |
| | 5′ labelled with 6-fluorescein amidite (FAM) | |
| | 3′ containing Black Hole Quencher (BHQ) | |

DMD exon 46

| | | |
|---|---|---|
| Forward primer | 5′-TTTATGGTTGGAGGAAGCAGA-3′ | 300 |
| | SEQ ID NO: 13 | |
| Reverse primer | 5′-AATGGGCAGAAAACCAATGA-3′ | 300 |
| | SEQ ID NO: 14 | |
| Probe | 5′-AACCTGGAAAAGAGCAGCAACT-3′ | 90 |
| | SEQ ID NO: 27 | |
| | 5′ labelled with YakimaYellow™ | |
| | 3′ containing Black Hole Quencher (BHQ) | |

DMD exon 50

| | | |
|---|---|---|
| Forward primer | 5′-CTGAGTGGAAGGCGGTAAAC-3′ | 300 |
| | SEQ ID NO: 15 | |
| Reverse primer | 5′-TCTCACCCAGTCATCACTTCA-3′ | 300 |
| | SEQ ID NO: 16 | |
| Probe | 5′-ACTTCAAGAGCTGAGGGCAAAG-3′ | 80 |
| | SEQ ID NO: 28 | |
| | 5′ labelled with ROX™ | |
| | 3′ containing Black Hole Quencher (BHQ) | |

DMD exon 52

| | | |
|---|---|---|
| Forward primer | 5′-AATACACAACGCTGAAGAACCC-3′ | 300 |
| | SEQ ID NO: 17 | |
| Reverse primer | 5′-TTGTGTGTCCCATGCTTGTT-3′ | 300 |
| | SEQ ID NO: 18 | |
| Probe | 5′-CGCTGCCCAAAATTTGAAAAA-3′ | 150 |
| | SEQ ID NO: 29 | |
| | 5′ labelled with Atto 647N | |
| | 3′ containing Black Hole Quencher (BHQ) | |

DMD exon 54

| | | |
|---|---|---|
| Forward primer | 5′-TCTATAGCAGTTGGCCAAAGAC-3′ | 300 |
| | SEQ ID NO: 19 | |
| Reverse primer | 5′-TCATGGTCCATCCAGTTTCA-3′ | 300 |
| | SEQ ID NO: 20 | |
| Probe | 5′-AATATCAATGCCTCTTGGAGAAGC-3′ | 180 |
| | SEQ ID NO: 30 | |
| | 5′ labelled with Atto 700 | |
| | 3 ′ containing Black Hole Quencher (BHQ) | |

120 samples with known genotypes were analysed. Deletion of any of the five target exons (exons 44, 46, 50, 52 and 54) of the DMD gene was identified by the absence of amplification of the corresponding target. As shown in Table 2, the assay correctly identified all subjects, with either presence or absence of amplification of the target exon. To corroborate this absence of amplification, for each exon, we divided the endpoint fluorescence of the sample by the median endpoint fluorescence of the corresponding exon of all samples of the microplate. A ratio below 0.2, for any exon, was considered as deleted.

TABLE 2
Amplification results of the different subject groups: “deleted group”, “carrier
group” and “control group”.

| | Target DMD exon | | | | | | |
|---|---|---|---|---|---|---|---|
| Subject | Exon | Exon | Exon | Exon | Exon | | Number |
| group | 44 | 46 | 50 | 52 | 54 | Genotype | of patients |
| Deleted group | Del* | Ampli * | Ampli | Ampli | Ampli | del_13-44 | n = 34 |
| Deleted group | Del | Ampli | Ampli | Ampli | Ampli | del_44 | (total) |
| Deleted group | Del | Ampli | Ampli | Ampli | Ampli | del_20-44 | |
| Deleted group | Del | Ampli | Ampli | Ampli | Ampli | del_20-44 | |
| Deleted group | Ampli | Del | Del | Del | Del | del_46-55 | |
| Deleted group | Ampli | Del | Del | Del | Del | del_45-55 | |
| Deleted group | Ampli | Del | Del | Del | Del | del_45-55 | |
| Deleted group | Ampli | Del | Del | Del | Del | del_45-55 | |
| Deleted group | Ampli | Del | Del | Del | Del | del_45-55 | |
| Deleted group | Ampli | Del | Del | Del | Ampli | del_46-53 | |
| Deleted group | Ampli | Del | Del | Del | Ampli | del 46-52 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Del | Ampli | Ampli | del_45-50 | |
| Deleted group | Ampli | Del | Ampli | Ampli | Ampli | del_45-48 | |
| Deleted group | Ampli | Del | Ampli | Ampli | Ampli | del_45-47 | |
| Deleted group | Ampli | Del | Ampli | Ampli | Ampli | del_45-47 | |
| Deleted group | Ampli | Del | Ampli | Ampli | Ampli | del_45-46 | |
| Deleted group | Ampli | Del | Ampli | Ampli | Ampli | del_46-48 | |
| Deleted group | Ampli | Del | Ampli | Ampli | Ampli | del_45-47 | |
| Deleted group | Ampli | Del | Ampli | Ampli | Ampli | del_45-48 | |
| Deleted group | Amnli | Amnli | Del | Del | Del | del_48-55 | |
| Deleted group | Ampli | Ampli | Del | Del | Del | del_49-54 | |
| Deleted group | Ampli | Ampli | Del | Del | Del | del_49-54 | |
| Deleted group | Ampli | Ampli | Del | Ampli | Ampli | del_50 | |
| Deleted group | Ampli | Ampli | Del | Ampli | Ampli | del_50 | |
| Deleted group | Ampli | Ampli | Del | Ampli | Ampli | del_50 | |
| Deleted group | Ampli | Ampli | Ampli | Del | Del | del_51-55 | |
| Deleted group | Ampli | Ampli | Ampli | Del | Del | del_51-64 | |
| Deleted group | Ampli | Ampli | Ampli | Del | Ampli | del_52 | |
| Deleted group | Ampli | Ampli | Ampli | Ampli | Ampli | N/A^{a} | n = 17 |
| Carrier group | Ampli | Ampli | Ampli | Ampli | Ampli | N/A^{b} | n = 32 |
| Carrier group | Ampli | Ampli | Ampli | Ampli | Ampli | N/A^{c} | n = 18 |
| Control group | Ampli | Ampli | Ampli | Ampli | Ampli | Normal. | n = 19 |

^{a}= male patients with a DMD deletion not overlapping any of the 5 target exons
^{b}= female subjects carrying a DMD deletion overlapping at least one of the 5 target exons
^{c}= female subjects carrying a DMD deletion not overlapping any of the 5 target exons
* Results show if the target exon is deleted (“Del”) or amplified (“Ampli”).
N/A: Not Applicable

Within the “deleted group” (n=51), there were 10 different deletion patterns seen: a deletion overlapping exon 44 (n=4 patients), a deletion overlapping exon 46 to 54 (n=5), a deletion overlapping exon 46 to 52 (n=2), a deletion overlapping exon 46 to 50 (n=7), a deletion overlapping exon 46 (n=7), a deletion overlapping exon 50 to 54 (n=3), a deletion overlapping exon 50 (n=3), a deletion overlapping exon 52 to 54 (n=2), a deletion overlapping exon 52 (n=1), and patients with a deletion not overlapping any target exon (n=17).

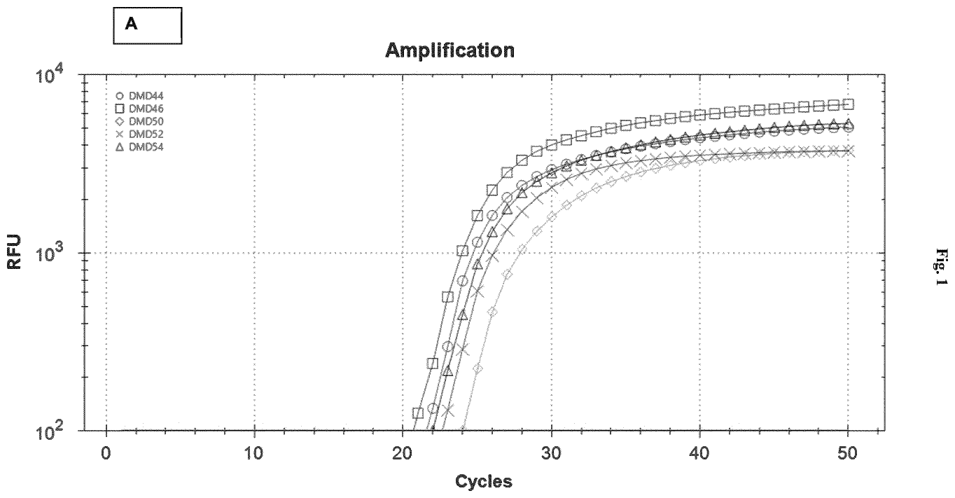

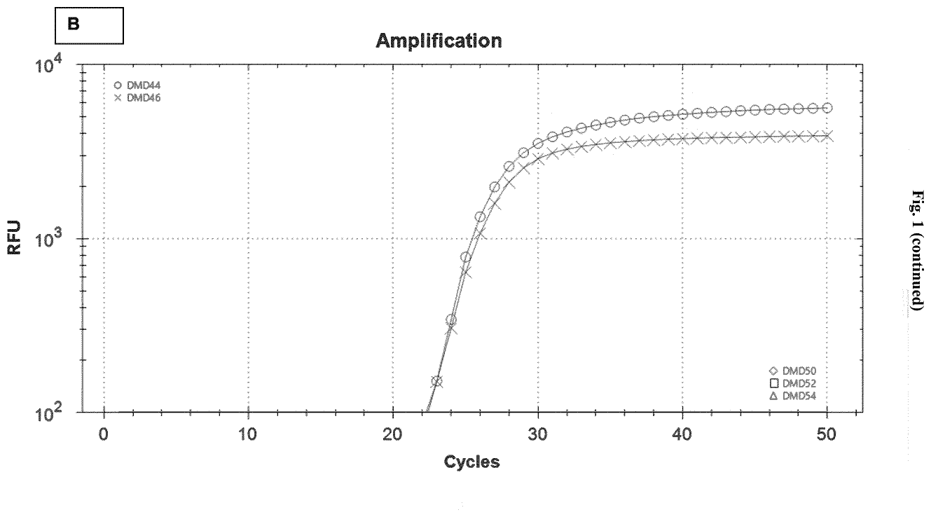

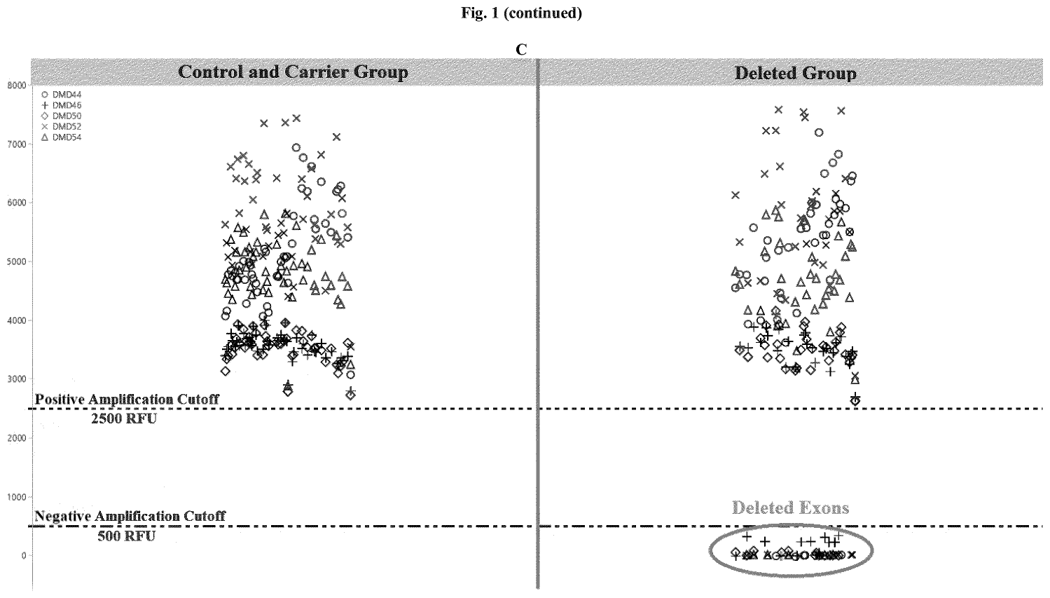

The 34 DMD patients of the “deleted group” with a deletion overlapping at least one of the five target exons were correctly characterised by an absence of fluorescence of the corresponding probes. The other 17 male patients of the “deleted group”, with a deletion of a DMD exon not targeted by our assay, presented a clear amplified profile of all target exons. All subjects of the “carrier group” and the “control group” also were characterized by a normal significant fluorescent signal of each probes. Our assay did not discriminate between carrier females and controls. The amplification profile of each target exon is summarized in FIG. 1. The results demonstrate that our technique reached 100% sensitivity and 100% specificity in the population studied.

### Example 2—DMD Genotyping Assay

### 2.1 Materials and Methods

### Subjects

Blood from DMD and control samples were collected as dried blood spots (DBS). DMD gene profile of all patients was performed using Multiplex Ligation-Dependent Probe Amplification (MLPA) assay in the course of diagnostic workup.

In total, 96 samples were collected and were classified into 2 different subgroups:

The “deleted group” consisted of 48 male patients with a clinical DMD phenotype. Amongst them, 28 had a confirmed deletion of one or more DMD exons amenable to exon-skipping targeted therapies (i.e. exons 7, 43, 45, 49 or 51). The other 20 male patients carried a deletion of another DMD exon, more particularly del_13-44, del_20-41, del_46-48, dup_02, del_50, del_50, dup_17-33, dup_17-33, dup_02, del_64-71, del_33-41, del_44, del_52, del_08-25, del_08-09, dup_10-26, trip_03-05, dup_02-07, dup_02-07 or del_10.

The “carrier group” included 48 females with one deleted DMD allele, of whom 32 carried a deletion that overlapped at least one of the target exons 7, 43, 45, 49 or 51, while 16 had a deletion of any other DMD exon. All females are heterozygous for DMD.

qPCR Technical Design

The DMD genotyping assay was designed and validated to detect hemi-(homo)-zygotic deletions of DMD exons 7, 43, 45, 49 and 51. The protocol uses a multiplex quantitative polymerase chain reaction (qPCR) assay.

DNA was extracted from one 3.1-mm dried blood spot according to the protocol described previously in Boemer F. et al. Newborn screening for SMA in Southern Belgium, Neuromuscul. Disord, 29, 343-349 (2019). Isolated DNA was not quantitated, and 1 μL of freshly extracted DNA was mixed with 5× Takyon master mix (Eurogentec, Liege, Belgium), primers and probes in a total volume of 25 μL. The sequences and concentrations of primers and probes are shown in Table 3.

Assays were run on a CFX96 Real-Time PCR System (Bio-Rad Laboratories, Hercules, Calif., USA) under the following conditions: 95° C. for 5 min, followed by 40 cycles of 95° C. for 30 sec and 61° C. for 75 sec.

### Interpretation of Findings

To normalize amplification results and to facilitate the interpretation, a normalized fluorescence ratio (NFR) was calculated dividing the endpoint fluorescence of each sample by the median endpoint fluorescence of the corresponding exon of all samples of the same run.

### Ethics

The study was approved by the local ethics committees and patients or guardians provided written informed consent (2019/278 and CEH 84/19). All experiments were performed in accordance with relevant guidelines and regulations.

### 2.2 Results

qPCR Results

Present inventors analyzed 96 samples with known genotypes. Deletion of any of the five target exons (exons 7, 43, 45, 49 and 51) of the DMD gene was characterized by the absence of amplification of the corresponding target. As shown in Table 4, the assay correctly identified all subjects, with either presence or absence of amplification of the target exon. To corroborate the absence of amplification, for each exon, an NFR below 0.2 was found as being discriminant between the deleted group and the carrier and control groups.

Within the “deleted group” (n=48), there were 8 different deletion patterns seen, including a deletion overlapping exon 7 (n=1 patients), a deletion overlapping exon 43 (n=2), a deletion overlapping at least exon 45 (n=16), a deletion overlapping at least exon 49 (n=15), a deletion overlapping at least exon 51 (n=12).

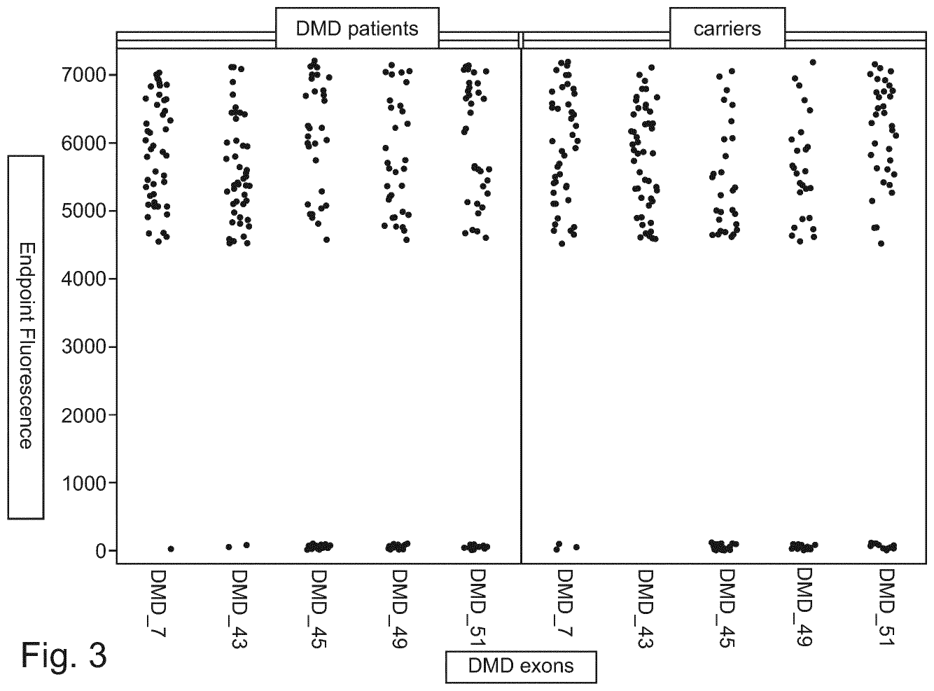

The 28 DMD patients of the “deleted group” with a deletion overlapping at least one of the five target exons were correctly characterized by an absence of fluorescence of the corresponding probes. The other 20 male patients of the “deleted group”, with a deletion of a DMD exon not targeted by present inventors' assay, presented a clear amplified profile of all target exons. All subjects of the female “carrier group” also were characterized by a normal significant fluorescent signal of each probe. Our assay did not discriminate between carrier females and controls. The amplification profile of each target exon is summarized in FIG. 3. The results demonstrate that the technique achieved 100% sensibility and 100% specificity in the population studied.

TABLE 3
qPCR primers and probes used to amplify and identify DMD Exons 7, 43, 45, 49 and 51.

| | | |
|---|---|---|
| DMD target | | Concentration |
| exon | Sequence | (nmol/L) |

DMD exon 7

| | | |
|---|---|---|
| Forward | 5′-TGT ATG TGT TTT AGG CCA GAC C-3′ | 300 |
| primer | SEQ ID NO: 46 | |
| Reverse | 5′-TCC AGA AAT TTA CCA ACC TTC A-3′ | 300 |
| primer | SEQ ID NO: 47 | |
| Probe | 5′- TGG AAT AGT GTG GTT TGC CAG C- | 130 |
| | 3′ | |
| | SEQ ID NO: 61 | |
| | 5′ labelled with 6-FAM | |
| | 3′ labelled with BHQ | |

DMD exon 43

| | | |
|---|---|---|
| Forward | 5′-AAA GCT CAG GTC GGA TTG AC-3′ | 300 |
| primer | SEQ ID NO: 48 | |
| Reverse | 5′-ACC TAC CCT TGT CGG TCC TT-3′ | 300 |
| primer | SEQ ID NO: 49 | |
| Probe | 5′-CCA GCT TGA TTT CCA ATG GG-3′ | 90 |
| | SEQ ID NO: 62 | |
| | 5′ labelled with Yakima Yellow | |
| | 3′ labelled with BHQ | |

DMD exon 45

| | | |
|---|---|---|
| Forward | 5′-GCC TTT TTG GTA TCT TAC AGG | 300 |
| primer | AAC-3′ | |
| | SEQ ID NO: 50 | |
| Reverse | 5′-CAG GCT TCC CAA TTT TTC C-3′ | 300 |
| primer | SEQ ID NO: 51 | |
| Probe | 5′-CAG AAC ATT GAA TGC AAC TGG G- | 80 |
| | 3′ | |
| | SEQ ID NO: 63 | |
| | 5′ labelled with ROX | |
| | 3′ labelled with BHQ | |

DMD exon 49

| | | |
|---|---|---|
| Forward | 5′-TTT TCC CCA GGA AAC TGA AA-3′ | 300 |
| primer | SEQ ID NO: 52 | |
| Reverse | 5′-CCC ATT ATG AGG TAA TGG ATA | 300 |
| primer | TTG-3′ | |
| | SEQ ID NO: 53 | |
| Probe | 5′-AAC CGG ATG TGG AAG AGA TTT | 150 |
| | TG-3′ | |
| | SEQ ID NO: 64 | |
| | 5′ labelled with Cy5 | |
| | 3′ labelled with BHQ | |

DMD exon 51

| | | |
|---|---|---|
| Forward | 5′-GAC TGT TAC TCT GGT GAC ACA | 300 |
| primer | ACC-3′ | |
| | SEQ ID NO: 54 | |
| Reverse | 5′-CAC CAT CAC CCT CTG TGA TTT-3′ | 300 |
| primer | SEQ ID NO: 55 | |
| Probe | 5′-GGG CTT GGA CAG AAC TTA CCG -3′ | 180 |
| | SEQ ID NO: 65 | |
| | 5′ labelled with Atto700 | |
| | 3′ labelled with BHQ | |

TABLE 4
Amplification results of the different subject groups: “deleted
group” and “carrier group”

| | Target DMD exon | | | | | | |
|---|---|---|---|---|---|---|---|
| Subject | Exon | Exon | Exon | Exon | Exon | | Number |
| group | 7 | 43 | 45 | 49 | 51 | Genotype^{d} | of patients |
| Deleted group | Amp * | Amp | Del* | Del | Del | del_45-55 | n = 28 |
| Deleted group | Amp | Amp | Del | Del | Del | del_45-55 | (total) |
| Deleted group | Amp | Amp | Del | Del | Del | del_45-55 | |
| Deleted group | Amp | Amp | Del | Del | Del | del_45-55 | |
| Deleted group | Amp | Amp | Del | Del | Amp | del_45-50 | |
| Deleted group | Amp | Amp | Del | Del | Amp | del_45-50 | |
| Deleted group | Amp | Amp | Del | Del | Amp | del_45-50 | |
| Deleted group | Amp | Amp | Del | Amp | Amp | del_45-48 | |
| Deleted group | Amp | Amp | Del | Del | Amp | del_45-50 | |
| Deleted group | Amp | Amp | Del | Del | Amp | del_45-50 | |
| Deleted group | Amp | Amp | Del | Amp | Amp | del_45-47 | |
| Deleted group | Amp | Amp | Del | Amp | Amp | del_45-47 | |
| Deleted group | Amp | Amp | Del | Amp | Amp | del_45-48 | |
| Deleted group | Amp | Amp | Del | Amp | Amp | del_45-47 | |
| Deleted group | Amp | Amp | Del | Amp | Amp | del_45-46 | |
| Deleted group | Amp | Amp | Del | Amp | Amp | del_45-47 | |
| Deleted group | Amp | Amp | Amp | Del | Del | del_48-55 | |
| Deleted group | Amp | Amp | Amp | Del | Del | del_49-54 | |
| Deleted group | Amp | Amp | Amp | Del | Del | del_49-54 | |
| Deleted group | Amp | Amp | Amp | Del | Del | del_46-53 | |
| Deleted group | Amp | Amp | Amp | Del | Del | del_46-55 | |
| Deleted group | Amp | Amp | Amp | Del | Amp | del_46-50 | |
| Deleted group | Amp | Amp | Amp | Amp | Del | del_51 | |
| Deleted group | Amp | Amp | Amp | Amp | Del | del_51-55 | |
| Deleted group | Amp | Amp | Amp | Amp | Del | del_51-64 | |
| Deleted group | Amp | Del | Amp | Amp | Amp | del_20-44 | |
| Deleted group | Amp | Del | Amp | Amp | Amp | del_20-44 | |
| Deleted group | Del | Amp | Amp | Amp | Amp | del_03-07 | |
| Deleted group | Amp | Amp | Amp | Amp | Amp | N/A^{a} | n = 20 |
| Carrier group | Amp | Amp | Amp | Amp | Amp | N/A^{b} | n = 32 |
| Carrier group | Amp | Amp | Amp | Amp | Amp | N/A^{c} | n = 16 |

^{a}= male patients with a DMD deletion not overlapping any of the 5 target exons.
^{b}= female subjects carrying a DMD deletion overlapping at least one of the 5 target exons.
^{c}= female subjects carrying a DMD deletion not overlapping any of the 5 target exons.
^{d}= Deleted exons were identified through MLPA. Intronic breakpoints have not been sequenced; genotypes are reported according to the LOVD-DMD database.
* Results show if the target exon is deleted (Del) or amplified (Amp).
N/A: not applicable

1. A method for analysing the Duchenne Muscular Dystrophy (DMD) gene in a sample containing genetic material of a subject, wherein the method detects the presence or absence of at least exons 44, 46, 50, 52 and 54 of the DMD gene in the genetic material of the subject, wherein the detection of the presence or absence of the exons comprises multiplex polymerase-based nucleic acid amplification;
wherein the polymerase-based nucleic acid amplification is configured to amplify:
a) to detect the presence or absence of exon 44 of the DMD gene, a target nucleic acid region using an amplification primer pair comprising an amplification primer configured to hybridise within the nucleic acid sequence GATCTGTCAAATCGCCTGCAGGTAAAAGC (SEQ ID NO: 1) and an amplification primer configured to hybridise within the nucleic acid sequence
(SEQ ID NO: 2)
TTCTTAAAGATCAGGTTCTGAAGGGTGATGGA;
b) to detect the presence or absence of exon 46 of the DMD gene, a target nucleic acid region using an amplification primer pair comprising an amplification primer configured to hybridise within the nucleic acid sequence TGTTATCTGCTTCCTCCAACCATAAAACAAA (SEQ ID NO: 3) and an amplification primer configured to hybridise within the nucleic acid sequence
(SEQ ID NO: 4)
TTCAATCATTGGTTTTCTGCCCATTAGGTT;
c) to detect the presence or absence of exon 50 of the DMD gene, a target nucleic acid region using an amplification primer pair comprising an amplification primer configured to hybridise within the nucleic acid sequence AAACGGTTTACCGCCTTCCACTCAGAGCTC (SEQ ID NO: 5) and an amplification primer configured to hybridise within the nucleic acid sequence
(SEQ ID NO: 6)
AACTATGAAGTGATGACTGGGTGAGAGAGAA;
d) to detect the presence or absence of exon 52 of the DMD gene, a target nucleic acid region using an amplification primer pair comprising an amplification primer configured to hybridise within the nucleic acid sequence TATCAGGGTTCTTCAGCGTTGTGTATTCCTTT (SEQ ID NO: 7) and an amplification primer configured to hybridise within the nucleic acid sequence TTTTTAACAAGCATGGGACACACAAAGCAA (SEQ ID NO: 8); and
e) to detect the presence or absence of exon 54 of the DMD gene, a target nucleic acid region using an amplification primer pair comprising an amplification primer configured to hybridise within the nucleic acid sequence CGGAGGTCTTTGGCCAACTGCTATAGATTTTT (SEQ ID NO: 9) and an amplification primer configured to hybridise within the nucleic acid sequence
(SEQ ID NO: 10)
GGTGGTGAAACTGGATGGACCATGAGGATT;
wherein the sample is dried blood; and
wherein the subject is a neonate.

2. The method according to claim 1, wherein the multiplex polymerase-based nucleic acid amplification is multiplex polymerase chain reaction (PCR).

3. The method according to claim 1, wherein the multiplex polymerase-based nucleic acid amplification is multiplex real-time quantitative amplification, preferably multiplex real-time quantitative PCR (qPCR).

4. The method according to claim 1, wherein the detection of the presence or absence of at least exons 44, 46, 50, 52 and 54 of the DMD gene in the genetic material of the subject is multiplexed in a single polymerase-based nucleic acid amplification reaction.

5. The method according to claim 1, wherein the polymerase-based nucleic acid amplification is configured to amplify:
a) to detect the presence or absence of exon 44 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence TACCTGCAGGCGATTTGAC (SEQ ID NO: 11) or of a sequence diverging from SEQ ID NO: 11 by addition, deletion or substitution of one or two nucleotides, or comprising or consisting of SEQ ID NO: 11; and
an amplification primer comprising at least 12 contiguous nucleotides, preferably at least 15 contiguous nucleotides, more preferably at least 18 contiguous nucleotides of the nucleic acid sequence CACCCTTCAGAACCTGATCTTT (SEQ ID NO: 12) or of a sequence diverging from SEQ ID NO: 12 by addition, deletion or substitution of one or two nucleotides, or comprising or consisting of SEQ ID NO: 12;
b) to detect the presence or absence of exon 46 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence TTTATGGTTGGAGGAAGCAGA (SEQ ID NO: 13) or of a sequence diverging from SEQ ID NO: 13 by addition, deletion or substitution of one or two nucleotides, or comprising or consisting of SEQ ID NO: 13; and
an amplification primer comprising at least 12 contiguous nucleotides, preferably at least 15 contiguous nucleotides, more preferably at least 18 contiguous nucleotides of the nucleic acid sequence AATGGGCAGAAAACCAATGA (SEQ ID NO: 14) of a sequence or diverging from SEQ ID NO: 14 by addition, deletion or substitution of one or two nucleotides, or comprising or consisting of SEQ ID NO: 14;
c) to detect the presence or absence of exon 50 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence CTGAGTGGAAGGCGGTAAAC (SEQ ID NO: 15) or of a sequence diverging from SEQ ID NO: 15 by addition, deletion or substitution of one or two nucleotides, or comprising or consisting of SEQ ID NO: 15; and
an amplification primer comprising at least 12 contiguous nucleotides, preferably at least 15 contiguous nucleotides, more preferably at least 18 contiguous nucleotides of the nucleic acid sequence TCTCACCCAGTCATCACTTCA (SEQ ID NO: 16) or of a sequence diverging from SEQ ID NO: 16 by addition, deletion or substitution of one or two nucleotides, or comprising or consisting of SEQ ID NO: 16;
d) to detect the presence or absence of exon 52 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence AATACACAACGCTGAAGAACCC (SEQ ID NO: 17) or of a sequence diverging from SEQ ID NO: 17 by addition, deletion or substitution of one or two nucleotides, or comprising or consisting of SEQ ID NO: 17; and
an amplification primer comprising at least 12 contiguous nucleotides, preferably at least 15 contiguous nucleotides, more preferably at least 18 contiguous nucleotides of the nucleic acid sequence TTGTGTGTCCCATGCTTGTT (SEQ ID NO: 18) or of a sequence diverging from SEQ ID NO: 18 by addition, deletion or substitution of one or two nucleotides, or comprising or consisting of SEQ ID NO: 18; and
e) to detect the presence or absence of exon 54 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence TCTATAGCAGTTGGCCAAAGAC (SEQ ID NO: 19) or of a sequence diverging from SEQ ID NO: 19 by addition, deletion or substitution of one or two nucleotides, or comprising or consisting of SEQ ID NO: 19; and
an amplification primer comprising at least 12 contiguous nucleotides, preferably at least 15 contiguous nucleotides, more preferably at least 18 contiguous nucleotides of the nucleic acid sequence TCATGGTCCATCCAGTTTCA (SEQ ID NO: 20) or of a sequence diverging from SEQ ID NO: 20 by addition, deletion or substitution of one or two nucleotides, or comprising or consisting of SEQ ID NO: 20.

6. The method according to claim 1, wherein the polymerase-based nucleic acid amplification is configured to amplify:
a) to detect the presence or absence of exon 44 of the DMD gene, a target nucleic acid region using an amplification primer pair comprising an amplification primer of nucleic acid sequence as set forth in SEQ ID NO: 11 and an amplification primer of nucleic acid sequence as set forth in SEQ ID NO: 12;
b) to detect the presence or absence of exon 46 of the DMD gene, a target nucleic acid region using an amplification primer pair comprising an amplification primer of nucleic acid sequence as set forth in SEQ ID NO: 13 and an amplification primer of nucleic acid sequence as set forth in SEQ ID NO: 14;
c) to detect the presence or absence of exon 50 of the DMD gene, a target nucleic acid region using an amplification primer pair comprising an amplification primer of nucleic acid sequence as set forth in SEQ ID NO: 15 and an amplification primer of nucleic acid sequence as set forth in SEQ ID NO: 16;
d) to detect the presence or absence of exon 52 of the DMD gene, a target nucleic acid region using an amplification primer pair comprising an amplification primer of nucleic acid sequence as set forth in SEQ ID NO: 17 and an amplification primer of nucleic acid sequence as set forth in SEQ ID NO: 18; and
e) to detect the presence or absence of exon 54 of the DMD gene, a target nucleic acid region using an amplification primer pair comprising an amplification primer of nucleic acid sequence as set forth in SEQ ID NO: 19 and an amplification primer of nucleic acid sequence as set forth in SEQ ID NO: 20.

7. The method according to claim 1, wherein the amplified target nucleic acid regions are detected by oligonucleotide probes configured to hybridise with said target nucleic acid regions, preferably wherein the oligonucleotide probes comprise detectable labels allowing for individual detection of each of the amplified target nucleic acid regions, more preferably wherein the detectable labels comprise distinct fluorophores having distinct excitation and/or emission characteristics, such that each of the amplified target nucleic acid regions can be individually detected by detecting the corresponding fluorophore.

8. The method according to claim 1, wherein:
a) to detect the presence or absence of exon 44 of the DMD gene, the oligonucleotide probe is configured to hybridise within the nucleic acid sequence
(SEQ ID NO: 21)
TTTAGCATGTTCCCAATTCTCAGGAATTTGTGTC;
b) to detect the presence or absence of exon 46 of the DMD gene, the oligonucleotide probe is configured to hybridise within the nucleic acid sequence
(SEQ ID NO: 22)
CTTTTAGTTGCTGCTCTTTTCCAGGTTCAAGT;
c) to detect the presence or absence of exon 50 of the DMD gene, the oligonucleotide probe is configured to hybridise within the nucleic acid sequence
(SEQ ID NO: 23)
GGCTGCTTTGCCCTCAGCTCTTGAAGTAAACG;
d) to detect the presence or absence of exon 52 of the DMD gene, the oligonucleotide probe is configured to hybridise within the nucleic acid sequence
(SEQ ID NO: 24)
TCTTGTTTTTCAAATTTTGGGCAGCGGTAAT;
e) to detect the presence or absence of exon 54 of the DMD gene, the oligonucleotide probe is configured to hybridise within the nucleic acid sequence
(SEQ ID NO: 25)
TGAATGCTTCTCCAAGAGGCATTGATATTCTCTG.

9. The method according to claim 1, wherein:
a) to detect the presence or absence of exon 44 of the DMD gene, the oligonucleotide probe comprises at least 12 contiguous nucleotides, preferably at least 15 contiguous nucleotides, more preferably at least 18 contiguous nucleotides of the nucleic acid sequence AAATTCCTGAGAATTGGGAACATG (SEQ ID NO: 26) or of a sequence diverging from SEQ ID NO: 26 by addition, deletion or substitution of one or two nucleotides, or comprises or consists of SEQ ID NO: 26, preferably the oligonucleotide probe is of nucleic acid sequence as set forth in SEQ ID NO: 26;
b) to detect the presence or absence of exon 46 of the DMD gene, the oligonucleotide probe comprises at least 12 contiguous nucleotides, preferably at least 15 contiguous nucleotides, more preferably at least 18 contiguous nucleotides of the nucleic acid sequence AACCTGGAAAAGAGCAGCAACT (SEQ ID NO: 27) or of a sequence diverging from SEQ ID NO: 27 by addition, deletion or substitution of one or two nucleotides, or comprises or consists of SEQ ID NO: 27, preferably the oligonucleotide probe is of nucleic acid sequence as set forth in SEQ ID NO: 27;
c) to detect the presence or absence of exon 50 of the DMD gene, the oligonucleotide probe comprises at least 12 contiguous nucleotides, preferably at least 15 contiguous nucleotides, more preferably at least 18 contiguous nucleotides of the nucleic acid sequence ACTTCAAGAGCTGAGGGCAAAG (SEQ ID NO: 28) or of a sequence diverging from SEQ ID NO: 28 by addition, deletion or substitution of one or two nucleotides, or comprises or consists of SEQ ID NO: 28, preferably the oligonucleotide probe is of nucleic acid sequence as set forth in SEQ ID NO: 28;
d) to detect the presence or absence of exon 52 of the DMD gene, the oligonucleotide probe comprises at least 12 contiguous nucleotides, preferably at least 15 contiguous nucleotides, more preferably at least 18 contiguous nucleotides of the nucleic acid sequence CGCTGCCCAAAATTTGAAAAA (SEQ ID NO: 29) or of a sequence diverging from SEQ ID NO: 29 by addition, deletion or substitution of one or two nucleotides, or comprises or consists of SEQ ID NO: 29, preferably the oligonucleotide probe is of nucleic acid sequence as set forth in SEQ ID NO: 29; and
e) to detect the presence or absence of exon 54 of the DMD gene, the oligonucleotide probe comprises at least 12 contiguous nucleotides, preferably at least 15 contiguous nucleotides, more preferably at least 18 contiguous nucleotides of the nucleic acid sequence AATATCAATGCCTCTTGGAGAAGC (SEQ ID NO: 30) or of a sequence diverging from SEQ ID NO: 30 by addition, deletion or substitution of one or two nucleotides, or comprises or consists of SEQ ID NO: 30, preferably the oligonucleotide probe is of nucleic acid sequence as set forth in SEQ ID NO: 30.

10. The method according to claim 1, wherein the subject is a mammal, preferably a human.

11. The method according to claim 1, wherein the sample is a dried blood spot.

12. A set of amplification primer pairs suitable for polymerase-based nucleic acid amplification, comprising an amplification primer pair configured to detect the presence or absence of exon 44 of the DMD gene, an amplification primer pair configured to detect the presence or absence of exon 46 of the DMD gene, an amplification primer pair configured to detect the presence or absence of exon 50 of the DMD gene, and an amplification primer pair configured to detect the presence or absence of exon 52 of the DMD gene, and an amplification primer pair configured to detect the presence or absence of exon 54 of the DMD gene, wherein the respective amplification primer pairs are as defined in any one of claim 1, 5 or 6.

13. The set of amplification primer pairs according to claim 12, further comprising a set of oligonucleotide probes configured to hybridise with the target nucleic acid regions, preferably wherein the respective oligonucleotide probes are as defined in any one of claims 7 to 9 and 22.

14. A set of oligonucleotide probes, wherein the respective oligonucleotide probes are as defined in any one of claims 7 to 9 and 22.

15. A composition comprising the set of amplification primer pairs and/or the set of oligonucleotide probes according to any one of claims 12 to 14 and 23.

16. A kit of parts comprising the set of amplification primer pairs and/or the set of oligonucleotide probes according to any one of claims 10 to 14 and 23, and optionally further comprising reagents sufficient for formulating a polymerase-based nucleic acid amplification reaction mixture.

17. The method according to claim 1, wherein the method further comprises detecting the presence or absence of exons 7, 43, 45, 49 and 51 of the DMD gene in the genetic material of the subject, and
wherein the polymerase-based nucleic acid amplification is further configured to amplify:
f) to detect the presence or absence of exon 7 of the DMD gene, a target nucleic acid region using an amplification primer pair comprising an amplification primer configured to hybridise within the nucleic acid sequence AAATAGGTCTGGCCTAAAACACATACACATAC (SEQ ID NO: 36) and an amplification primer configured to hybridise within the nucleic acid sequence
(SEQ ID NO: 37)
GATCCTGAAGGTTGGTAAATTTCTGGACTACC;
g) to detect the presence or absence of exon 43 of the DMD gene, a target nucleic acid region using an amplification primer pair comprising an amplification primer configured to hybridise within the nucleic acid sequence ATAATGTCAATCCGACCTGAGCTTTGTTGT (SEQ ID NO: 38) and an amplification primer configured to hybridise within the nucleic acid sequence
(SEQ ID NO: 39)
TGTACAAGGACCGACAAGGGTAGGTAACAC;
h) to detect the presence or absence of exon 45 of the DMD gene, a target nucleic acid region using an amplification primer pair comprising an amplification primer configured to hybridise within the nucleic acid sequence CTGGAGTTCCTGTAAGATACCAAAAAGGCAAAAC (SEQ ID NO: 40) and an amplification primer configured to hybridise within the nucleic acid sequence
(SEQ ID NO: 41)
CTACAGGAAAAATTGGGAAGCCTGAATCT;
i) to detect the presence or absence of exon 49 of the DMD gene, a target nucleic acid region using an amplification primer pair comprising an amplification primer configured to hybridise within the nucleic acid sequence TGCTATTTCAGTTTCCTGGGGAAAAGAACC(SEQ ID NO: 42) and an amplification primer configured to hybridise within the nucleic acid sequence TCTAGCAATATCCATTACCTCATAATGGGTTATG (SEQ ID NO: 43); and
j) to detect the presence or absence of exon 51 of the DMD gene, a target nucleic acid region using an amplification primer pair comprising an amplification primer configured to hybridise within the nucleic acid sequence CCACAGGTTGTGTCACCAGAGTAACAGTCTGAGT (SEQ ID NO: 44) and an amplification primer configured to hybridise within the nucleic acid sequence
(SEQ ID NO: 45)
TTATAAAATCACAGAGGGTGATGGTGGGTGA.

18. The method according to claim 1, wherein the detection of the presence or absence of at least exons 7, 43, 44, 45, 46, 49, 50, 51, 52 and 54 of the DMD gene in the genetic material of the subject is multiplexed in a single polymerase-based nucleic acid amplification reaction.

19. The method according to claim 17, wherein the polymerase-based nucleic acid amplification is further configured to amplify:
f) to detect the presence or absence of exon 7 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence TGTATGTGTTTTAGGCCAGACC (SEQ ID NO: 46) or of a sequence diverging from SEQ ID NO: 46 by addition, deletion or substitution of one or two nucleotides, or comprising or consisting of SEQ ID NO: 46; and
an amplification primer comprising at least 12 contiguous nucleotides, preferably at least 15 contiguous nucleotides, more preferably at least 18 contiguous nucleotides of the nucleic acid sequence TCCAGAAATTTACCAACCTTCA (SEQ ID NO: 47) or of a sequence diverging from SEQ ID NO: 47 by addition, deletion or substitution of one or two nucleotides, or comprising or consisting of SEQ ID NO: 47;
g) to detect the presence or absence of exon 43 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence AAAGCTCAGGTCGGATTGAC (SEQ ID NO: 48) or of a sequence diverging from SEQ ID NO: 48 by addition, deletion or substitution of one or two nucleotides, or comprising or consisting of SEQ ID NO: 48; and
an amplification primer comprising at least 12 contiguous nucleotides, preferably at least 15 contiguous nucleotides, more preferably at least 18 contiguous nucleotides of the nucleic acid sequence ACCTACCCTTGTCGGTCCTT (SEQ ID NO: 49) or of a sequence diverging from SEQ ID NO: 49 by addition, deletion or substitution of one or two nucleotides, or comprising or consisting of SEQ ID NO: 49;
h) to detect the presence or absence of exon 45 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence GCCTTTTTGGTATCTTACAGGAAC (SEQ ID NO: 50) or of a sequence diverging from SEQ ID NO: 50 by addition, deletion or substitution of one or two nucleotides, or comprising or consisting of SEQ ID NO: 50; and
an amplification primer comprising at least 12 contiguous nucleotides, preferably at least 15 contiguous nucleotides, more preferably at least 18 contiguous nucleotides of the nucleic acid sequence CAGGCTTCCCAATTTTTCC (SEQ ID NO: 51) or of a sequence diverging from SEQ ID NO: 51 by addition, deletion or substitution of one or two nucleotides, or comprising or consisting of SEQ ID NO: 51;
i) to detect the presence or absence of exon 49 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence TTTTCCCCAGGAAACTGAAA (SEQ ID NO: 52) or of a sequence diverging from SEQ ID NO: 52 by addition, deletion or substitution of one or two nucleotides, or comprising or consisting of SEQ ID NO: 52; and
an amplification primer comprising at least 12 contiguous nucleotides, preferably at least 15 contiguous nucleotides, more preferably at least 18 contiguous nucleotides of the nucleic acid sequence CCCATTATGAGGTAATGGATATTG (SEQ ID NO: 53) or of a sequence diverging from SEQ ID NO: 53 by addition, deletion or substitution of one or two nucleotides, or comprising or consisting of SEQ ID NO: 53; and
j) to detect the presence or absence of exon 51 of the DMD gene, a target nucleic acid region using an amplification primer pair comprising:
an amplification primer comprising at least 12 contiguous nucleotides, preferably at least 15 contiguous nucleotides, more preferably at least 18 contiguous nucleotides of the nucleic acid sequence GACTGTTACTCTGGTGACACAACC (SEQ ID NO: 54) or of a sequence diverging from SEQ ID NO: 54 by addition, deletion or substitution of one or two nucleotides, or comprising or consisting of SEQ ID NO: 54; and
an amplification primer comprising at least 12 contiguous nucleotides, preferably at least 15 contiguous nucleotides, more preferably at least 18 contiguous nucleotides of the nucleic acid sequence CACCATCACCCTCTGTGATTT (SEQ ID NO: 55) or of a sequence diverging from SEQ ID NO: 55 by addition, deletion or substitution of one or two nucleotides, or comprising or consisting of SEQ ID NO: 55.

20. The method according to claim 17, wherein the polymerase-based nucleic acid amplification is further configured to amplify:
f) to detect the presence or absence of exon 7 of the DMD gene, a target nucleic acid region using an amplification primer pair comprising an amplification primer of nucleic acid sequence as set forth in SEQ ID NO: 46 and an amplification primer of nucleic acid sequence as set forth in SEQ ID NO: 47;
g) to detect the presence or absence of exon 43 of the DMD gene, a target nucleic acid region using an amplification primer pair comprising an amplification primer of nucleic acid sequence as set forth in SEQ ID NO: 48 and an amplification primer of nucleic acid sequence as set forth in SEQ ID NO: 49;
h) to detect the presence or absence of exon 45 of the DMD gene, a target nucleic acid region using an amplification primer pair comprising an amplification primer of nucleic acid sequence as set forth in SEQ ID NO: 50 and an amplification primer of nucleic acid sequence as set forth in SEQ ID NO: 51;
i) to detect the presence or absence of exon 49 of the DMD gene, a target nucleic acid region using an amplification primer pair comprising an amplification primer of nucleic acid sequence as set forth in SEQ ID NO: 52 and an amplification primer of nucleic acid sequence as set forth in SEQ ID NO: 53; and
j) to detect the presence or absence of exon 51 of the DMD gene, a target nucleic acid region using an amplification primer pair comprising an amplification primer of nucleic acid sequence as set forth in SEQ ID NO: 54 and an amplification primer of nucleic acid sequence as set forth in SEQ ID NO: 55.

21. The method according to claim 17, wherein:
f) to detect the presence or absence of exon 7 of the DMD gene, the oligonucleotide probe is configured to hybridise within the nucleic acid sequence
(SEQ ID NO: 56)
TGACTGCTGGCAAACCACACTATTCCAGTCAA;
g) to detect the presence or absence of exon 43 of the DMD gene, the oligonucleotide probe is configured to hybridise within the nucleic acid sequence
(SEQ ID NO: 57)
TTTTTCCCATTGGAAATCAAGCTGGGAGAG;
h) to detect the presence or absence of exon 45 of the DMD gene, the oligonucleotide probe is configured to hybridise within the nucleic acid sequence
(SEQ ID NO: 58)
TCTTCCCCAGTTGCATTCAATGTTCTGACAAC;
i) to detect the presence or absence of exon 49 of the DMD gene, the oligonucleotide probe is configured to hybridise within the nucleic acid sequence
(SEQ ID NO: 59)
TTAGACAAAATCTCTTCCACATCCGGTTGTTTA;
j) to detect the presence or absence of exon 51 of the DMD gene, the oligonucleotide probe is configured to hybridise within the nucleic acid sequence
(SEQ ID NO: 60)
CCAGTCGGTAAGTTCTGTCCAAGCCCGGTTG.

22. The method according to claim 17, wherein:
f) to detect the presence or absence of exon 7 of the DMD gene, the oligonucleotide probe comprises at least 12 contiguous nucleotides, preferably at least 15 contiguous nucleotides, more preferably at least 18 contiguous nucleotides of the nucleic acid sequence TGGAATAGTGTGGTTTGCCAGC (SEQ ID NO: 61) or of a sequence diverging from SEQ ID NO: 61 by addition, deletion or substitution of one or two nucleotides, or comprises or consists of SEQ ID NO: 61, preferably the oligonucleotide probe is of nucleic acid sequence as set forth in SEQ ID NO: 61;
g) to detect the presence or absence of exon 43 of the DMD gene, the oligonucleotide probe comprises at least 12 contiguous nucleotides, preferably at least 15 contiguous nucleotides, more preferably at least 18 contiguous nucleotides of the nucleic acid sequence CCAGCTTGATTTCCAATGGG (SEQ ID NO: 62) or of a sequence diverging from SEQ ID NO: 62 by addition, deletion or substitution of one or two nucleotides, or comprises or consists of SEQ ID NO: 62, preferably the oligonucleotide probe is of nucleic acid sequence as set forth in SEQ ID NO: 62;
h) to detect the presence or absence of exon 45 of the DMD gene, the oligonucleotide probe comprises at least 12 contiguous nucleotides, preferably at least 15 contiguous nucleotides, more preferably at least 18 contiguous nucleotides of the nucleic acid sequence CAGAACATTGAATGCAACTGGG (SEQ ID NO: 63) or of a sequence diverging from SEQ ID NO: 63 by addition, deletion or substitution of one or two nucleotides, or comprises or consists of SEQ ID NO: 63, preferably the oligonucleotide probe is of nucleic acid sequence as set forth in SEQ ID NO: 63;
i) to detect the presence or absence of exon 49 of the DMD gene, the oligonucleotide probe comprises at least 12 contiguous nucleotides, preferably at least 15 contiguous nucleotides, more preferably at least 18 contiguous nucleotides of the nucleic acid sequence AACCGGATGTGGAAGAGATTTTG (SEQ ID NO: 64) or of a sequence diverging from SEQ ID NO: 64 by addition, deletion or substitution of one or two nucleotides, or comprises or consists of SEQ ID NO: 64, preferably the oligonucleotide probe is of nucleic acid sequence as set forth in SEQ ID NO: 64; and
j) to detect the presence or absence of exon 51 of the DMD gene, the oligonucleotide probe comprises at least 12 contiguous nucleotides, preferably at least 15 contiguous nucleotides, more preferably at least 18 contiguous nucleotides of the nucleic acid sequence GGGCTTGGACAGAACTTACCG (SEQ ID NO: 65) or of a sequence diverging from SEQ ID NO: 65 by addition, deletion or substitution of one or two nucleotides, or comprises or consists of SEQ ID NO: 65, preferably the oligonucleotide probe is of nucleic acid sequence as set forth in SEQ ID NO: 65.

23. The set of amplification primer pairs according to claim 12, further comprising an amplification primer pair configured to detect the presence or absence of exon 7 of the DMD gene, an amplification primer pair configured to detect the presence or absence of exon 43 of the DMD gene, an amplification primer pair configured to detect the presence or absence of exon 45 of the DMD gene, an amplification primer pair configured to detect the presence or absence of exon 49 of the DMD gene, an amplification primer pair configured to detect the presence or absence of exon 51 of the DMD gene, wherein the respective amplification primer pairs are as defined in any one of claim 17, 19 or 20.
